(19) United States
(12) Patent Application Publication
(10) Pub. No.: US 2005/0054752 A1
(43) Pub. Date: Mar. 10, 2005

(54) Peptide-based diblock and triblock dispersants and diblock polymers

(76) Inventors: John P. O'Brien, Oxford, PA (US); Jianjun Yang, Hockessin, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/935,254

(22) Filed: Sep. 7, 2004

Related U.S. Application Data
(60) Provisional application No. 60/501,497, filed on Sep. 8, 2003.

(51) Int. Cl. C03C 17/00; C09D 5/00; C07K 2/00

(52) U.S. Cl. 523/160; 523/161; 530/300

(57) ABSTRACT

Peptides have been identified that bind with high affinity to common ink pigments and to various print media, including paper and fabrics. These peptides were used to prepare diblock and triblock dispersants for coating applications, including ink jet printing, and diblock polymers for treating paper and fabrics. The diblock dispersants consist of a pigment-binding peptide coupled to a hydrophilic linker, a pigment-binding peptide coupled to a print medium-binding peptide or a print medium-binding peptide coupled to a hydrophobic linker. The diblock polymers consist of a print medium-binding peptide coupled to a hydrophilic linker or a benefit agent. The triblock dispersants consist of a pigment-binding peptide coupled to a hydrophilic linker, which is coupled to a print medium-binding peptide.

This patent application claims the benefit of U.S. Provisional Patent Application, 60/501497, filed Sep. 8, 2003.

### FIELD OF THE INVENTION

The invention relates to the field of dispersants for particulate solids, especially pigments. More specifically, the invention relates to diblock and triblock dispersants and diblock polymers comprising specific peptide sequences.

### BACKGROUND OF THE INVENTION

Polymeric dispersants are widely used to stabilize pigments in coating systems such as paints and finishes, and in ink jet printing inks (Reuter et al., Progress in Organic Coatings 37:161-167 (1999), Schmitz et al, Progress in Organic Coatings 35:191-196 (1999), and Spinelli, Adv. Mater. 10:1215-1218 (1998)). The dispersant serves to form a shell around the pigment particle, preventing flocculation and coagulation. In aqueous systems, the pigment dispersion is generally stabilized by either a nonionic or ionic technique. In the non-ionic technique, the pigment particles are stabilized by a polymer that has a water-soluble, hydrophilic section that extends into the water and provides entropic or steric stabilization. Representative polymers useful for this purpose include polyvinyl alcohol, cellulosics, and ethylene oxide modified phenols. While the non-ionic technique is not sensitive to pH changes or ionic contamination, it has a major disadvantage for many applications in that the final product is water sensitive. Thus, if used in ink applications or the like, the pigment will tend to smear upon exposure to moisture.

In the ionic technique, the pigment particles are stabilized by a polymer of an ion containing monomer, such as neutralized acrylic, maleic, or vinyl sulfonic acid. The polymer provides stabilization through a charged double layer mechanism whereby ionic repulsion hinders the particles from flocculation. Since the neutralizing component tends to evaporate after application, the polymer then has reduced water solubility and the final product is not water sensitive. Polymer dispersants, such as block and graft polymers, that provide both steric and ionic stabilization make the most robust pigment dispersions (Spinelli, supra).

Polymer dispersants having both random and block structures have been disclosed. For example, Ohta et al. in U.S. Pat. No. 4,597,794 disclose a random polymer dispersant having ionic hydrophilic segments and aromatic hydrophobic segments that adhere to the pigment surface. Ma et al. in U.S. Pat. No. 5,085,698 disclose the use of AB or BAB block copolymers as dispersants for aqueous ink jet inks. The A segment is a hydrophobic homopolymer or copolymer that serves to bind to the pigment particle and the B segment is a hydrophilic polymer, or salt thereof, that serves to disperse the pigment in the aqueous medium. Ma et al. in U.S. Pat. No. 5,519,085 disclose an ABC triblock polymer dispersant, wherein the A segment is a hydrophilic polymer that serves to facilitate dispersion of the pigment in water, the B segment is a polymer capable of binding to the pigment, and the C segment is a hydrophilic or hydrophobic polymer that serves to stabilize the dispersion. A combination of polymer dispersants may also be used, as described by Rose et al. in GB 2349153. While these random and block polymer dispersants offer good stability for the dispersed pigment, further improvements are desired for more high quality coating applications. For example, dispersants having a stronger interaction with the pigment would improve the stability of the dispersion. Moreover, a dispersant with a stronger interaction with the coating substrate would result in a more durable coating. This is particularly important for textile printing where enhanced durability is required.

The use of proteins and peptides as dispersants is also known in the art. For example, Brueckmann et al. in U.S. Pat. No. 5,124,438 describe the use of chemically modified proteins, such as casein, collagen, albumin and gelatin, as dispersants in color formulations. Garris et al. (Colloids and Surfaces A: Physicochemical and Engineering Aspects 80:103-112 (1993)) describe the use of polyanionic amino acid peptides such as polyaspartate as dispersants for inorganic mineral particles. However, there has been no suggestion in the art of the use of specific binding peptide sequences in diblock and triblock dispersants.

Various enzymes, such as cellulases, xylanases, mannanases, arabinofuranosidases, acetyl esterases, and chitinases, are known to contain domains that specifically bind to cellulose. Based on their amino acid sequences, these cellulose binding domains (CBDs) have been divided into several different families (Linder et al., J. Biotechnol. 57:12-28 (1997)). It is known that CBDs rely on several aromatic amino acids for binding to cellulose surfaces and that CBDs typically range in size from 33 to 168 amino acid residues. Because of their cellulose-binding property, CBDs have been used in compositions for treating fabrics to ensure the deposition of a benefit agent onto the fabric. For example, Pedersen et al. in WO 9740127 describe a hybrid enzyme containing a phenol oxidizing enzyme linked to a CBD for treating fabric to produce a bleached look. Von der Osten et al. in U.S. Pat. No. 6015783 describe a process for removal or bleaching of stains from cellulosic fabric using non-cellulolytic enzymes, such as amylases, lipases, pectinases and oxidoreductases, linked to a CBD. Jones et al. in WO 9800500 and Smets et al. in WO 01/18897 describe compositions comprising a CBD linked to a benefit agent, such as softening agents, perfumes, antioxidants, polymeric lubricants, dye fixative agents, and soil repelling and release agents, for treating cellulosic fabrics.

Mimic CBDs, which are synthetic peptides of 30 or fewer amino acids, preferably containing at least three aromatic amino acids, have also been described (Bjorkquist et al., WO 0132848). The preferred sequences given for the mimic CBDs in that disclosure are (AW)₄K₂, given as SEQ ID NO:1, and (WE)₄K₂, given as SEQ ID NO:2. Fabric care compositions comprising the mimic CBDs linked to various benefit agents, in order to improve deposition onto the fabric surface, are taught by Bjorkquist et al. supra. However, there is no description in the art of the use of CBDs or mimic CBDs in diblock or triblock dispersants.

Since its introduction in 1985, phage display has been widely used to discover a variety of ligands including peptides, proteins and small molecules for drug targets (Dixit, J. of Sci. & Ind. Research, 57:173-183 (1998)). The applications have expanded to other areas such as studying protein folding, novel catalytic activities, DNA-binding proteins with novel specificities, and novel peptide-based biomaterial scaffolds for tissue engineering (Hoess, Chem. Rev. 101:3205-3218 (2001) and Holmes, Trends Biotechnol. 20:16-21 (2002)). Whaley et al. (Nature 405:665-668 (2000)) disclose the use of phage display screening to identify peptide sequences that can bind specifically to different crystallographic forms of inorganic semiconductor substrates. The use of phage display to identify peptides that specifically bind carbon-based nanostructures is described by Jagota et al. (copending U.S. patent application Ser. No. 10/453415; WO 03/102020). Although the method of generating large, diverse peptide libraries with phage display has been known for some time, it has not been applied to the problem of finding peptides that specifically bind to pigments for use in making improved dispersants. Nolan et al. (WO 00/23463 and Chemistry and Biology 5:731-728 (1998)) disclose peptides that bind with high affinity to fluorescent dyes, such as fluorescein, Oregon Green 514, Rhodamine Red and Texas Red, which were identified using phage display screening. However, these are small, soluble dye molecules which do not serve as pigments for coating applications.

Han et al. (Shengwu Huaxue Yu Shengwu Wuli Xuebao 30:263-266 (1998)) describe the identification of peptides that specifically bind to a cellulose matrix using the phage display method. The deduced amino acid sequences of these cellulose binding peptides have a conserved aromatic residue, tyrosine or phenylalanine, which is similar to the normal cellulose binding domain of cellulose-binding proteins. The amino acid sequence SWYL, given as SEQ ID NO:3, was identified as a good candidate for a cellulose binding motif. The use of these cellulose-binding peptides in new dispersants for coating applications is not described in that disclosure.

Estell et al. in WO 01/79479 describe a modified phage display screening method that comprises contacting a peptide library with an anti-target to remove peptides that bind to the anti-target, then contacting the non-binding peptides with the target. Using this method, peptide sequences that bind to collar soil, but not to polyester/cotton and peptide sequences that bind to polyurethane, but not to cotton, polyester, or polyester/cotton fabrics were identified. No peptide sequences that bind to fabrics are reported in that disclosure.

Nomoto et al in EP1275728 describe the identification of pigment-binding peptides using phage display. Some carbon black, copper phthalocyanine, titanium dioxide, and silicon dioxide-binding peptide sequences are disclosed. In that disclosure, the pigment-binding peptides are used to immobilize the enzyme polyhydroxyalkanoate synthase onto the pigment particles. The binding peptide sequences are incorporated into a recombinant fusion protein using genetic engineering. The immobilized enzyme catalyzes the formation of polyhydroxyalkanoate on the pigment particle, forming an encapsulated particle. Peptide-based diblock and triblock pigment dispersants are not described in that disclosure.

Several of the peptide binding sequences of the instant invention have been reported for other purposes. Engler et al. (WO 02/044329 and Eur. J. Biochem. 268:2004-2012 (2001)) disclose the peptide sequence given by SEQ ID NO:4, identified by phage display screening as a binding peptide to human transferrin receptor. Wolcke et al. (Nucleotides and Nucleic Acids 20:1239-1241 (2001)) identified the peptide sequence given as SEQ ID NO:5 as a DNA binding peptide using phage display screening. Jouault et al. (Glycobiology 11:693-701 (2001)) identified the peptide sequence given as SEQ ID NO:6 as a peptide that mimics Candida albicans-derived β-1,2-linked mannosides using phage display screening. McDonald et al. (WO 00/032631) identified the peptide sequence given as SEQ ID NO: 22 as a peptide that binds to angiogenesis-inhibiting proteins. Masuda et al. (WO 00/070035) disclose the peptide sequence given by SEQ ID NO:30, identified using phage display screening as a peptide that binds specifically to the N-terminal region of a retrovirus integrase. Zhang et al. (Shengwu Huaxue Yu Shengwu Wuli Xuebao 32:475-479 (2000)) disclose the peptide sequence given by SEQ ID NO:33, identified using phage display screening as a peptide that binds to the tyrosine kinase receptor EphB2. None of these disclosures teach the use of these peptide sequences in new dispersants for coating applications.

In view of the above, a need exists for dispersants which provide improved stability of the dispersed pigment to meet the demanding needs of more advanced high quality coating applications, including commercial ink jet printers. A need also exists for ink dispersants that provide improved durability on various print media, particularly textile fabrics.

Applicants have met the stated needs by identifying peptide sequences using phage display screening that specifically bind to pigments and to various print media with high affinity and using them to design diblock and triblock dispersants and diblock polymers by coupling the peptides to various linker blocks.

### SUMMARY OF THE INVENTION

The invention provides a number of peptides, selected by combinatorial biopanning means, that demonstrate specific affinity for various pigments and print media, particularly cellulose and polyester. The peptides have been assembled into various diblock, triblock and dispersant configurations optionally linked with a variety of hydrophilic and hydrophobic linkers. These compositions have utility for attaching pigments and dyes to print media in a selective fashion.

Accordingly the invention provides a pigment-binding peptide selected from the group consisting of SEQ ID NO:7, 8, 9, 10, 14, 15, 16, 17, 18, 19, 20, 21, 23, 24, 25, 26, 27, 28, and 29.

Similarly the invention provides a print medium-binding peptide selected from the group consisting of SEQ ID NO: 31, 32, 34, 35, 36, 37, 38, and 40.

The pigment binding peptides are preferably selected by a process comprising the steps of:
(i) providing a library of combinatorial generated peptides;
(ii) contacting the library of (i) with a pigment to form a reaction solution comprising:
   (A) peptide-pigment complexes;
   (B) unbound pigment; and
   (C) uncomplexed peptides;
(iii) isolating the peptide-pigment complexes of (ii); and
(iv) eluting the peptides from the peptide complex of (ii).

In similar fashion the print-media binding peptides are preferably selected by a process comprising the steps of:
(i) providing a library of combinatorial generated peptides;
(ii) contacting the library of (i) with a pigment to form areaction solution comprising:
   (A) peptide-pigment complexes;
   (B) unbound pigment; and
   (C) uncomplexed peptides;
(iii) isolating the peptide-pigment complexes complexes of (ii); and
(iv) eluting the peptides from the peptide complex of (ii).

In one preferred embodiment a diblock dispersant is provided having the general structure: [PBP]ₙ—HL, wherein
a) PBP is a pigment-binding peptide;
b) HL is a hydrophilic linker; and
c) n ranges from 1 to about 5.

In another preferred embodiment the invention provides a diblock polymer having the general structure: [PMBP]ₙ—HL, wherein
a) PMBP is a print medium-binding peptide;
b) HL is a hydrophilic linker; and
c) n ranges from 1 to about 5.

In an alternate embodiment the invention provides a diblock dispersant having the general structure: [PBP]ₙ—[PMBP]ₙ, wherein
a) PBP is a pigment-binding peptide;
b) PMBP is a print medium-binding peptide; and
c) n ranges from I to about 5.

In another embodiment the invention provides a diblock dispersant having the general structure: [PMBP]ₙ—HPL, wherein,
a) PMBP is a print medium-binding peptide;
b) HPL is a hydrophobic linker; and
c) n ranges from 1 to about 5.

In an alternate embodiment the invention provides a diblock polymer having the general structure: [PMBP]ₙ—BA, wherein,
a) PMBP is a print medium-binding peptide selected from the group consisting of SEQ ID NO:, 4, 30, 31, 32, 33, 34, 35, 36, 37, 38, and 40;
b) BA is a benefit agent; and
c) n ranges from 1 to about 5.

In an other preferred embodiment the invention provides a triblock dispersant having the general structure: [PBP]ₙ—HL—[PMBP]ₙ, wherein
a) PBP is a pigment-binding peptide;
b) HL is a hydrophilic linker;
c) PMBP is a print medium-binding peptide; and
d) n ranges from 1 to about 5.

In another embodiment the invention provides an aqueous ink composition comprising:
a) an aqueous carrier medium;
b) a pigment; and
c) the dispersant of the invention.

In similar fashion the invention provides a water-born coating composition comprising:
a) an aqueous carrier medium;
b) a pigment;
c) a film-forming resin; and
d) the dispersant or polymer of the invention.

Alternatively the invention provides a pigmented plastic composition comprising:
a) a plastic resin;
b) a pigment; and
c) the dispersant or polymer of the invention

Additionally the invention provides a method for recovering and purifying a recombinant protein comprising:
a) providing a sample comprising a recombinant protein containing a cellulose-binding peptide sequence selected from the group consisting of SEQ ID NO:35, 36, 37, and 38;
b) contacting the sample with a cellulose support;
c) separating the cellulose support from the sample; and
d) recovering the recombinant protein by treating the support with an eluting agent.

Methods are also provided for recovering and purifying a recombinant protein comprising:
a) providing a sample comprising a recombinant protein containing a pigment-binding peptide sequence selected from the group-consisting of SEQ ID NO:5, 6, 7, 8, 9, 10, 14, 15, 16, 17, 18, 19, 20, 21, 23, 24, 25, 26, 27, 28, and 29;
b) contacting the sample with a pigment;
c) separating the pigment from the sample; and
d) recovering the recombinant protein by treating the pigment with an eluting agent.

Additionally methods are provided for affixing a pigment to a print medium comprising:
a) providing a triblock dispersant of the invention having a pigment-binding peptide and a print-media-binding-peptide;
b) contacting the triblock dispersant of (a) with a pigment wherein the pigment complexes with the pigment-binding-peptide to form a peptide complexed triblock: and
c) contacting the peptide complexed triblock of step (c) with a print-medium wherein the print-medium complexes with the print-media-binding-peptides and the pigment is affixed to the print medium.

### BRIEF DESCRIPTION OF THE FIGURES AND SEQUENCE DESCRIPTIONS

The invention can be more fully understood from the following detailed description, figures and the accompanying sequence descriptions, which form a part of this application.

FIG. 1 is a gel image of the SDS-PAGE of fractions from the purification of the recombinant triblock protein dispersants (A) 6H-TBP1 and (B) 6H-TBP2.

FIG. 2 shows the results of carbon black pigment dispersion by the triblock protein TBP251.

FIG. 3 shows the results of pigment adhesion testing mediated by triblock protein TBP251.

The following sequences conform with 37 C.F.R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs:1-3 are the amino acid sequences of mimic CBD peptides.

SEQ ID NO:4 is the amino acid sequence of a paper-binding peptide of the present invention.

SEQ ID NO:5 is the amino acid sequence of a pigment-binding peptide of the present invention.

SEQ ID NO:6 is the amino acid sequence of a carbon black-binding peptide and a cellulose-binding peptide of the present invention.

SEQ ID NOs:7-29 are the amino acid sequences of pigment-binding peptides of the present invention.

SEQ ID NOs:30-40 are the amino acid sequences of the print media-binding peptides of the present invention.

SEQ ID NO:41 is the oligonucleotide primer used to sequence phage DNA.

SEQ ID NOs:42-44 are the amino acid sequences of peptides used as controls in the ELISA binding assay.

SEQ ID NO:45 is the amino acid sequence of the interdomain linker in cellobiohydrolase I of the plant pathogen fungus Aspergillus used as the ST-linker.

SEQ ID NOs:46-50 are oligonucleotides used to prepare the TBP1 gene.

SEQ ID NO:51 is the nucleotide sequence of the TPB1 gene.

SEQ ID NO:52 is the amino acid sequence of the triblock protein dispersant designated as TPB1.

SEQ ID NOs:53,54 are oligonucleotide primers used to amplify the TBP1 fragment.

SEQ ID NO:55 is the amino acid sequence of the 6H-TBP1 protein.

SEQ ID NO:56 is the amino acid sequence of the interdomain linker in endoglucanase A of the bacterium Cellulomonas fimi used as the PT-linker.

SEQ ID NOs:57-61 are oligonucleotides used to prepare the TBP2 gene.

SEQ ID NO:62 is the nucleotide sequence of the TPB2 gene.

SEQ ID NO:63 is the amino acid sequence of the triblock protein dispersant designated as TPB2.

SEQ ID NOs:64,65 are oligonucleotide primers used to amplify the TBP2 fragment.

SEQ ID NO:66 is the amino acid sequence of the 6H-TBP2 protein.

SEQ ID NOs: 67-114 are the nucleotide sequences of the oligonucleotides used to prepare the plasmids in Example 6.

SEQ ID NO:115 is the amino acid sequence of the cotton-binding peptide used to prepare the diblock dispersant of Example 13.

SEQ ID NO:116 is the nucleotide sequence of the coding region of the Gateway entrance plasmid pENTR-TBP101, as described in Example 19.

SEQ ID NO:117 is the amino acid sequence of a tetra-peptide which represents a Xa recognition site.

SEQ ID NO: 118 is the amino acid sequence of the protein encoded by Gateway entrance plasmid pENTR-TBP101.

SEQ ID NO:119 is the nucleotide sequence of coding region of the Gateway entrance plasmid pENTR-TBP201, as described in Example 19.

SEQ ID NO: 120 is the amino acid sequence of the protein encoded by Gateway entrance plasmid pENTR-TBP201.

SEQ ID NO:121 is the nucleotide sequence of expression plasmid plNK251.

SEQ ID NO:122 is the amino acid sequence of the protein encoded by expression plasmid plNK251.

### DETAILED DESCRIPTION OF THE INVENTION

The present invention provides peptide sequences that specifically bind to various-pigments or print media. Additionally, the present invention provides peptide-based diblock and triblock dispersants for use in ink jet printing inks, and other coating systems, such as paints and finishes. The diblock dispersants of the present invention comprise a specific pigment-binding peptide coupled to a hydrophilic linker, a print medium-binding peptide coupled to a hydrophobic linker, or a pigment-binding peptide coupled to a print medium-binding peptide. The triblock dispersants of the present invention comprise a specific pigment-binding peptide coupled to a hydrophilic linker, which is coupled to a specific print medium-binding peptide. The present invention also provides diblock polymers comprising a print medium-binding peptide coupled to a hydrophilic linker or a benefit agent, which are useful for treating paper or fabrics.

The following definitions are used herein and should be referred to for interpretation of the Claims and the specification.

“CBD” means cellulose binding domain.

“PBP” means pigment-binding peptide.

“PMBP” means print medium-binding peptide.

“HL” means hydrophilic linker.

“HPL” means hydrophobic linker.

“BA” means benefit agent. The term “benefit agent” as used herein refers to any chemical compound that provides a desirable effect on a fiber, fabric or surface.

The term “peptide” refers to two or more amino acids joined to each other by peptide bonds or modified peptide bonds. As used herein, the terms “peptide”, “polypeptide”, and “protein” are used interchangeably.

The term “pigment” refers to an insoluble, organic or inorganic colorant.

The term “print medium” refers to any substrate suitable for ink jet printing.

The term “dispersant” as used herein refers to a substance that stabilizes the formation of a colloidal solution of solid pigment particles in a liquid medium.

The term “diblock dispersant” as used herein refers to a pigment dispersant that consists of two different units or blocks, each serving a specific function. The peptide-based diblock dispersants of the present invention consist of a pigment-binding peptide block coupled to a hydrophilic linker block, a pigment-binding peptide block coupled to a print medium-binding peptide block, or a hydrophobic linker block coupled to a print medium-binding peptide block. The diblock dispersant may contain multiple copies of any of the peptide blocks.

The term “diblock polymer” as used herein refers to a composition that consists of two different units or blocks, each serving a specific function. The peptide-based diblock polymers of the present invention consist of a print medium-binding peptide block coupled to a hydrophilic linker block, or a print medium-binding peptide block coupled to a benefit agent. The diblock polymer may contain multiple copies of the peptide block.

The term “triblock dispersant” as used herein refers to a pigment dispersant that consists of three different units or blocks, each serving a specific function. The peptide-based triblock dispersant of the present invention consists of a pigment-binding peptide block, a hydrophilic linker block, and a print-medium peptide block. The triblock dispersant may contain multiple copies of any of the peptide blocks.

The term “stringency” as it is applied to the selection of the pigment-binding peptides and print medium-binding peptides of the present invention, refers to the concentration of the eluting agent (usually detergent) used to elute peptides from the pigment or print medium. Higher concentrations of the eluting agent provide more stringent conditions.

The term “peptide-pigment complex” means structure comprising a peptide bound to a pigment particle via a binding site on the peptide.

The term “peptide-print medium complex” means structure comprising a peptide bound to a print medium via a binding site on the peptide.

The term “MB₅₀” refers to the concentration of the binding peptide that gives a signal that is 50% of the maximum signal (Bₘₐₓ) obtained in an ELISA-based binding assay, as described in Example 6. The MB₅₀ provides an indication of the strength of the binding interaction or affinity of the components of the complex. The lower the value of MB₅₀, the stronger the interaction of the peptide with its corresponding substrate.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences “Chimeric gene” refers to any gene that is not a native gene, comprising regulatory and coding sequences that are not found together in nature. Accordingly, a chimeric gene may comprise regulatory sequences and coding sequences, that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “expression”, as used herein, refers to the transcription and stable accumulation of sense (mRNA) or antisense RNA derived from the nucleic acid fragment of the invention. Expression may also refer to translation of mRNA into a polypeptide.

The term “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The term “host cell” refers to cell which has been transformed or transfected, or is capable of transformation or transfection by an exogenous polynucleotide sequence.

The terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

The term “phage” or “bacteriophage” refers to a virus that infects bacteria. Altered forms may be used for the purpose of the present invention. The preferred bacteriophage is derived from the “wild” phage, called M13. The M13 system can grow inside a bacterium, so that it does not destroy the cell it infects but causes it to make new phages continuously. It is a single-stranded DNA phage.

The term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

Standard recombinant DNA and molecular cloning techniques used herein are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Press, Spring Harbor, N.Y. (1984); and by Ausubel, F. M. et al., Current Protocols in Molecular Biology, published by Greene Publishing Assoc. and Wiley-Interscience (1987).

The present invention comprises specific pigment and print medium-binding peptides and their use in diblock and triblock dispersants for ink jet inks and other coating applications.

Pigments

As used herein, the term “pigment” means an insoluble colorant. A wide variety of organic and inorganic pigments alone or in combination may be used in the present invention. Examples of organic pigments include, but are not limited to Cyan, Yellow, Red, Blue, Orange, Magenta, Black, Green, Violet, Light Cyan, and Light Magenta. Preferred organic pigments are carbon black, such as Carbon Black FW18, and colored pigments such as Cromophthal® Yellow 131AK (Ciba Specialty Chemicals), Sunfast® Magenta 122 (Sun Chemical) and Sunfast® Blue 15:3 (Sun Chemical). Examples of inorganic pigments include, but are not limited to finely divided metals, such as copper, iron, aluminum, and alloys thereof; and metal oxides, such as silica, alumina, and titania. Additional examples of suitable pigments are given by Ma et al. in U.S. Pat. No. 5,085,698, incorporated herein by reference.

Print Media

The term “print medium” as used herein is any substrate suitable for ink jet printing. Suitable print media include, but are not limited to, printing paper, sheets, films, nonwovens and textile fabrics, such as polyester, nylon, Lycra®, silk, cotton, cotton blends, rayon, flax, linen, wool, spandex, acetate, acrylic, modacrylic, aramid and polyolefin. These print media are readily available from a number of commercial sources.

Pigment-Binding and Print Medium-Binding Peptides

Pigment-binding peptides (PBPs) and print medium-binding peptides (PMBPs) as defined herein are peptide sequences that specifically bind with high affinity to pigments and print media, respectively. The pigment-binding peptides and the print medium-binding peptides of the present invention are from about 5 amino acids to 20 amino acids, more preferably, from about 7 amino acids to about 12 amino acids in length.

Suitable pigment-binding peptide and print medium-binding peptide sequences may be selected using methods that are well known in the art. The peptides of the present invention are generated randomly and then selected against a specific pigment or a specific print medium based upon their binding affinity for the substrate of interest. The generation of random libraries of peptides is well known and may be accomplished by a variety of techniques including, bacterial display (Kemp, D. J.; Proc. Natl. Acad. Sci. USA 78(7):4520-4524 (1981), and Helfman et al., Proc. Natl. Acad. Sci. USA 80(1):31-35, (1983)), yeast display (Chien et al., Proc Natl Acad Sci USA 88(21):9578-82 (1991)), combinatorial solid phase peptide synthesis (U.S. Pat. No. 5,449,754, U.S. Pat. No. 5,480,971, U.S. Pat. No. 5,585,275, U.S. Pat. No.5,639,603), and phage display technology (U.S. Pat. No. 5,223,409, U.S. Pat. No. 5,403,484, U.S. Pat. No. 5,571,698, U.S. Pat. No. 5,837,500). Techniques to generate such biological peptide libraries are described in Dani, M., J. of Receptor & Signal Transduction Res., 21 (4):447468 (2001).

A preferred method to randomly generate peptides is by phage display. Phage display is an in vitro selection technique in which a peptide or protein is genetically fused to a coat protein of a bacteriophage, resulting in display of fused peptide on the exterior of the phage virion, while the DNA encoding the fusion resides within the virion. This physical linkage between the displayed peptide and the DNA encoding it allows screening of vast numbers of variants of peptides, each linked to a corresponding DNA sequence, by a simple in vitro selection procedure called “biopanning”. In its simplest form, biopanning is carried out by incubating the pool of phage-displayed variants with a target of interest that has been immobilized on a plate or bead, washing away unbound phage, and eluting specifically bound phage by disrupting the binding interactions between the phage and the target. The eluted phage is then amplified in vivo and the process is repeated, resulting in a stepwise enrichment of the phage pool in favor of the tightest binding sequences. After 3 or more rounds of selection/amplification, individual clones are characterized by DNA sequencing.

After a suitable library of peptides has been generated, they are then contacted with an appropriate amount of the test substrate, specifically a selected pigment or a specific print medium. The test substrate is presented to the library of peptides while suspended in solution. A preferred solution is a buffered aqueous saline solution containing a surfactant. A suitable solution is Tris-buffered saline (TBS) with 0.1% Tween® 20. The solution may additionally be agitated by any means in order to increase the mass transfer rate of the peptides to the pigment or print medium surface, thereby shortening the time required to attain maximum binding.

Upon contact, a number of the randomly generated peptides will bind to the pigment or print medium substrate to form a peptide-substrate complex. Unbound peptide may be removed by washing. After all unbound material is removed, peptides having varying degrees of binding affinities for the test substrate may be fractionated by selected washings in buffers having varying stringencies. Increasing the stringency of the buffer used increases the required strength of the bond between the peptide and substrate in the peptide-substrate complex.

A number of substances may be used to vary the stringency of the buffer solution in peptide selection including, but not limited to, acidic pH (1.5-3.0); basic pH (10-12.5); high salt concentrations such as MgCl₂ (3-5 M) and LiCl (5-10 M); water; ethylene glycol (25-50%); dioxane (5-20%); thiocyanate (1-5 M); guanidine (2-5 M); urea (2-8 M); and various concentrations of different surfactants such as SDS (sodium dodecyl sulfate), DOC (sodium deoxycholate), Nonidet P40, Triton X-100, Tween® 20, wherein Tween® 20 is preferred. These substances may be prepared in buffer solutions including, but not limited to, Tris-HCl, Tris-buffered saline, Tris-borate, Tris-acetic acid, triethylamine, phosphate buffer, and glycine-HCl, wherein Tris-buffered saline solution is preferred.

It will be appreciated that peptides having increasing binding affinities for the pigment or print medium substrates may be eluted by repeating the selection process using buffers with increasing stringencies. The eluted peptides can be identified and sequenced by any means known in the art.

Thus it is an object of the present invention to provide a process for generating a pigment-binding peptide or a print medium-binding peptide comprising:
a) providing a library of combinatorial generated peptides;
b) contacting the library of (a) with a pigment or a print medium to form a reaction solution comprising:
   (i) peptide-pigment or peptide-print medium complexes;
   (ii) unbound pigment or print medium; and
   (iii) uncomplexed peptides;
c) isolating the peptide-pigment or peptide-print medium complexes of (b); and
d) eluting the peptides from the peptide complex of (b), wherein the eluted peptide has specific binding properties.

Pigment-binding peptides and print medium-binding peptides have been identified using the above process. The pigment-binding peptides of the invention comprise at least about 40 mole % of the amino acids: glycine, alanine, valine, leucine, isoleucine, methionine, proline, phenylalanine, and tryptophan. Specifically, binding peptides were isolated that have a high affinity for the pigments carbon black, given as SEQ ID NOs:6-9, Cromophthal®-Yellow, given as SEQ ID NOs:5, 10-17, Sunfast® Magenta, given as SEQ ID NOs:18-20, and Sunfast® Blue, given as SEQ ID NOs:5, 21-29. The cellulose-binding peptides of the invention comprise at least about 14 mole % of the amino acids: serine, threonine and tyrosine. Binding peptides having a high binding affinity for cellulose (a major component of cotton) include SEQ ID NOs:35-39. The polyester-binding peptides of the invention comprise at least about 20 mole % of the amino acids: phenylalanine, tryptophan, and tyrosine. Binding peptides having a high affinity for polyester (poly(ethylene terephthalate)) include SEQ ID NO:40. Additionally, binding peptides were isolated that have a binding affinity for the following print media: cotton, given as SEQ ID NOs:30-31, polyester/cotton, given as SEQ ID NOs:30 and 32, and printing paper, given as SEQ ID NOs:4, 30, 33, and 34.

The binding energy, herein defined as the amount of energy released upon the interaction of the binding peptide with its respective substrate in units of kilocalories per mole (kcal/mol), provides an indication of the strength of the binding affinity. The molar heat of adsorption for the interaction of the pigment-binding peptides and the print medium-binding peptides of the present invention with their respective substrate may be used as a measure of the binding energy of the interaction. The pigment-binding peptides and the print medium-binding peptides of the present invention have an exothermic molar heat of adsorption in water of at least 20 kcal/mol, as measured by flow microcalorimetry. The molar heats of adsorption may be measured using standard calorimetric measurement techniques using a flow microcalorimeter, such as that available from Microscal, London, Ltd, as described in detail in Example 7. The measurement of the molar heat of adsorption of the binding peptide with its respective substrate is accomplished by passing a solution containing a known concentration of the binding peptide in water over a known amount of its respective substrate in the flow microcalorimeter and measuring the amount of heat released as a result of the binding interaction by means of thermistors contained in the microcalorimeter. The mass transfer value, defined herein as the amount of the binding peptide adsorbed per unit area of substrate, may be determined by means of a mass sensitive detector, such as a refractometer, located downstream of the flow microcalorimeter. The molar heat of adsorption may then be calculated by dividing the heat of adsorption obtained from the microcalorimetry determination by the mass transfer value. For an exothermic process, the molar heat of adsorption is given as a negative value to indicate that heat is released in the process.

Production of Binding Peptides

The binding peptides of the present invention may be prepared using standard peptide synthesis methods, which are well known in the art (see for example Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, the peptides of the present invention may be prepared using recombinant DNA and molecular cloning techniques. Genes encoding the pigment or print medium-binding peptides may be produced in heterologous host cells, particularly in the cells of microbial hosts.

Preferred heterologous host cells for expression of the binding peptides of the present invention are microbial hosts that can be found broadly within the fungal or bacterial families and which grow over a wide range of temperature, pH values, and solvent tolerances. Because transcription, translation, and the protein biosynthetic apparatus are the same irrespective of the cellular feedstock, functional genes are expressed irrespective of carbon feedstock used to generate cellular biomass. Examples of host strains include, but are not limited to, fungal or yeast species such as Aspergillus, Trichoderma, Saccharomyces, Pichia, Candida, Hansenula, or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella.

A variety of expression systems can be used to produce the peptides of the present invention. Such vectors include, but are not limited to, chromosomal, episomal and virus-derived vectors, e.g., vectors derived from bacterial plasmids, from bacteriophage, from transposons, from insertion elements, from yeast episoms, from viruses such as baculoviruses, retroviruses and vectors derived from combinations thereof such as those derived from plasmid and bacteriophage genetic elements, such as cosmids and phagemids. The expression system constructs may contain regulatory regions that regulate as well as engender expression. In general, any system or vector suitable to maintain, propagate or express polynucleotide or polypeptide in a host cell may be used for expression in this regard. Microbial expression systems and expression vectors contain regulatory sequences that direct high level expression of foreign proteins relative to the growth of the host cell. Regulatory sequences are well known to those skilled in the art and examples include, but are not limited to, those which cause the expression of a gene to be turned on or off in response to a chemical or physical stimulus, including the presence of regulatory elements in the vector, for example, enhancer sequences. Any of these could be used to construct chimeric genes for production of any of the binding peptides of the present invention. These chimeric genes could then be introduced into appropriate microorganisms via transformation to provide high level expression of the peptides.

Vectors or cassettes useful for the transformation of suitable host cells are well known in the art. Typically the vector or cassette contains sequences directing transcription and translation of the relevant gene, one or more selectable markers, and sequences allowing autonomous replication or chromosomal integration. Suitable vectors comprise a region 5′ of the gene, which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. Selectable marker genes provide a phenotypic trait for selection of the transformed host cells such as tetracycline or ampicillin resistance in E. coli.

Initiation control regions or promoters which are useful to drive expression of the chimeric gene in the desired host cell are numerous and familiar to those skilled in the art. Virtually any promoter capable of driving the gene is suitable for producing the binding peptides of the present invention including, but not limited to: CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, ara, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

The vector containing the appropriate DNA sequence as described supra, as well as an appropriate promoter or control sequence, may be employed to transform an appropriate host to permit the host to express the peptide of the present invention. Cell-free translation systems can also be employed to produce such peptides using RNAs derived from the DNA constructs of the present invention. Optionally it may be desired to produce the instant gene product as a secretion product of the transformed host. Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal which is functional in the production host. Methods for choosing appropriate signal sequences are well known in the art (see for example EP 546049 and WO 9324631). The secretion signal DNA or facilitator may be located between the expression-controlling DNA and the instant gene or gene fragment, and in the same reading frame with the latter.

Diblock Dispersants and Diblock Polymers

In one embodiment of the present invention, the diblock dispersant consists of a pigment-binding peptide (PBP) block coupled to a hydrophilic linker (HL) block. Preferably, the peptide is covalently attached to the hydrophilic linker. The function of the pigment-binding peptide is to bind strongly to the pigment particles, thus forming a stable dispersion. The hydrophilic linker extends into the aqueous phase and provides steric and ionic stabilization of the pigment particle against flocculation. The pigment-binding peptides include, but are not limited to, the peptides given by SEQ ID NOs:5-29, most preferably peptides given by SEQ ID NOs:5-7, 9, 10, 14-18, 20, 21, 25, 26, 28, and 29.

The hydrophilic linker may be a peptide composed of amino acids including, but not limited to the hydrophilic amino acids: Asp, Asn, Glu, Gln, His, Arg, Lys, Pro, serine, threonine, and mixtures thereof. The preferred peptide linkers are composed of the amino acids Pro, Glu, Lys and mixtures thereof. The peptide linker may be from 1 to about 80 amino acids, preferably from about 3 to about 50 amino acids. One skilled in the art will recognize that the ability of any given diblock combination to behave as an effective dispersant is strongly dependent upon the overall dispersion formulation, including additives, pH and the degree of neutralization of acid or base groups on the peptides, including the peptide linker. Therefore, the optimum length of the peptide linker for any particular application must be determined by routine experimentation. In addition to these simple diblock dispersants, it may be desirable to have multiple pigment-binding peptides coupled to the hydrophilic linker to enhance the interaction between the peptide and the pigment. These dispersants have the general structure [PBP]ₙ—HL, where n ranges from 1 to about 5, more preferably n is 1 to about 3.

These diblock peptide dispersants may be prepared using any method known in the art. For example, the pigment-binding peptide may be prepared using standard peptide synthesis methods described supra and then the peptide linker may be added by the in situ polymerization of the N-carboxyanhydride of the desired amino acid(s) via activation through the N-terminal amine of the pigment-binding peptide. This approach is based on the known ability of primary amines to catalyze the ring opening polymerization of N-carboxyanhydrides (see for example, Penczek, Models of Ring Opening Polymerization, CRC Press, Boca Raton, Fla. (1989)). In addition, the peptide blocks may be combined using carbodiimide coupling agents (see for example, Hermanson, Bioconjugate Techniques, Academic Press, New York (1996)), diacid chlorides, diisocyanates and other difunctional coupling reagents that are reactive to terminal amine and/or carboxylic acid terminal groups on the peptides. Alternatively, the entire diblock peptide dispersant may be prepared using the recombinant DNA and molecular cloning techniques described supra.

The hydrophilic linker may also be an organic polymer. Suitable synthetic organic polymers are characterized by having one or more moities that can covalently bind to the pigment-binding peptide. Preferably, these organic polymer linkers have a molecular weight of less that 10,000, more preferably between about 500 and 4000. Suitable synthetic organic polymer linkers include, but are not limited to, polyethylene glycol derivatives, polyacrylic acid/maleic acid polymers, derivatives of polyacrylic acid including its salts, polyvinyl alcohol, polyacrylamide, polymethylvinyl ether, cellulosic polymers and other polysaccharides, such as starches and glycogen. For example, many suitable polyethylene glycol (PEG) derivatives for use in the present invention are commercially available. Examples of these PEG derivatives include carboxyl, amino, hydroxysuccinimide esters and aldehydes. Polyacrylic acid polymers and derivatives, maleic acid polymers and mixtures thereof have carboxylic acid moities that may be covalently attached to an amine group on the pigment-binding peptide, using for example carbodiimide coupling. Polyvinyl alcohol polymers may be linked to the carboxylic acid group of aspartic or glutamic acid residues on the pigment-binding peptide via an esterification reaction. Suitable cellulosic polymers include methyl cellulose, hydroxypropyl cellulose, hydroxyethyl cellulose, and hydroxymethyl cellulose. For these cellulosic polymers and other polysaccharides, linkage to the peptide may be through esterification using the hydroxyl groups on cellulosic side chain and the carboxylic acid groups on peptide or by diisocyanate coupling through peptide primary amine groups and the cellulosic hydroxyl groups.

The hydrophilic linker may also be a combination of a peptide linker and a synthetic organic polymer, which may be prepared using the methods described above.

In another embodiment of the present invention, the diblock polymer consists of a print medium-binding peptide (PMBP) block coupled to a hydrophilic linker (HL) block. Preferably, the peptide is covalently attached to the hydrophilic linker. The function of the print medium-binding peptide is to bind strongly to the print medium surface, while the hydrophilic linker renders the surface hydrophilic. The print media binding peptides include, but are not limited, to the peptides given by SEQ ID NOs:4, 30-40, most preferably peptides given by SEQ ID NOs:4, 30-38, and 40. These PMBP—HL diblock polymers have application in coating compositions to provide beneficial surface properties to the treated surface. These improved surface properties include wetting and sheeting, soil removal, anti-soil deposition, and anti-spotting.

The hydrophilic linker may be a peptide, a synthetic organic polymer, or a mixture thereof, as described above for the PBP—HL diblock dispersants. PMBP—HL diblock polymers may be prepared using the methods described above for the PBP—HL diblock dispersants. In addition to these simple diblock polymers, it may be desirable to have multiple print medium-binding peptides coupled to the hydrophilic linker to enhance the interaction between the peptide and the print medium. These diblock polymers have the general structure [PMBP]ₙ—HL, where n ranges from 1 to about 5, more preferably n is 1 to about 3.

Additionally, the PMBP may be coupled to agents that confer functional benefits to a fabric or paper surface. The PMBP may be directly coupled to the benefit agent or the coupling may be through a linker. Any of the linkers described above may be used. Suitable benefit agents are well known in the art (see for example Bjorkquist et al., WO 01/32848, Jones et al, WO 98/00500, and Smets et al., WO 00/18897). Examples of benefit agents include, but are not limited, to perfumes, hygiene agents, insect control agents, enzymes, softening protein, fabric softening agents, soil release agents, bleaching agents, dye fixative agents, brighteners, antimicrobial agents, surfactants and mixtures thereof. Preferred benefit agents are fragrance and antimicrobial agents. These benefit agents may be coupled to the PMBPs using the methods described supra. These PMBP-benefit agent (BA) diblock polymers have the general structure [PMBP]ₙ—BA, where n ranges from 1 to about 5, more preferably n is 1 to about 3.

In another embodiment of the present invention the diblock dispersant consists of a pigment-binding peptide block coupled to a print medium-binding peptide block, preferably via a covalent bond. The function of the pigment-binding peptide is to bind strongly to the pigment particles, thus forming a stable dispersion. The function of the print medium-binding peptide is to bind strongly to the print medium substrate, thus enhancing the durability of the coating. These PBP—PMBP diblock dispersants may be prepared using the pigment binding peptides and the print medium-binding peptides listed above and coupling them together using any method known in the art. For example, the two peptides may be covalently coupled using a cross-linking agent such as 4,4′ methylenebis(phenylisocyanate). The two peptides may also be coupled using carbodiimide coupling agents, diacid chlorides, diisocyanates and other difunctional coupling reagents that are reactive to terminal amine and/or carboxylic acid terminal groups on the peptides. Alternatively, the PBP—PMBP diblock dispersant may be prepared using the recombinant DNA and molecular cloning techniques described supra. In addition to these simple diblock dispersants, it may be desirable to have multiple PBPs coupled to the multiple PMBPs. These dispersants have the general structure [PBP]ₙ—[PMBP]ₙ, where n ranges from 1 to about 5, more preferably n is 1 to about 3.

In yet another embodiment of the present invention, the diblock dispersant consists of a print medium-binding peptide block coupled to a hydrophobic linker (HPL) block, preferably via a covalent bond. In this diblock dispersant, the print medium-binding peptide serves two functions, specifically, binding strongly to the surface of the print medium and functioning as a hydrophilic linker to disperse the pigment. The hydrophobic linker block serves to interact with the pigment particle. The print media-binding peptides include, but are not limited, to the peptides given by SEQ ID NOs:4, 6, 30-40, most preferably peptides given by SEQ ID NOs:4, 30-38, and 40.

The hydrophobic linker may be a peptide, a synthetic organic polymer, or a mixture thereof. The peptide-based hydrophobic linker may be a peptide composed of amino acids including, but not limited to the hydrophobic amino acids: Ala, Gly, Met, Leu, Ile, Val, Cys, Phe, Tyr, Trp, and mixtures thereof. Additionally, the peptide-based hydrophobic linker may contain hydrophobic derivatives of a hydrophilic amino acid, for example, benzyl glutamate. The peptide linker may be from 1 to about 80 amino acids, preferably from about 5 to about 30 amino acids.

The hydrophobic linker may also be a hydrophobic, synthetic organic polymer. The hydrophobic linker is water insoluble and has a molecular weight of at least about 300. Suitable hydrophobic, synthetic organic polymer linkers are described by Ma et al. in U.S. Pat. No. 5,085,698, incorporated herein by reference. Preferred hydrophobic, synthetic organic polymers are homopolymers and copolymers prepared from methacrylate, butyl methacrylate, 2-ethylhexyl methacrylate, benzylmethacrylate, or copolymers of methyl methacrylate with butyl methacrylate.

These PMBP—HPL diblock dispersants may be prepared by the methods described above for the other diblock dispersants. In addition to these simple diblock dispersants, it may be desirable to have multiple PMBPs coupled to the hydrophobic linker. These dispersants have the general structure [PMBP]ₙ—HPL, where n ranges from 1 to about 5, more preferably n is 1 to about 3.

Triblock Dispersants

In a preferred embodiment of the present invention, the dispersant is a triblock consisting of a pigment-binding peptide (PBP) block coupled to a hydrophilic linker (HL) block, which is coupled to a print medium-binding peptide block. Preferably, the blocks are covalently attached to each other. The function of the pigment-binding peptide is to bind strongly to the pigment particles, thus forming a stable dispersion. The hydrophilic linker extends into the aqueous phase and provides steric and ionic stabilization of the pigment particle against flocculation and provides a more accessible presentation of the PMBP from the pigment surface. The function of the print medium-binding peptide is to bind strongly to the print medium substrate, thus enhancing the durability of the coating. Suitable pigment-binding peptides, hydrophilic linkers, and print medium-binding peptides are the same as those described supra for the diblock dispersants. Additionally, mimic CBDs, such as those given by SEQ ID NOs:1-3, may be used as the PMBP in these triblock dispersants. In addition to the simple triblock dispersants, it may be desirable to have dispersants containing multiple PBPs and PMBPs to enhance the strength of the interaction of the dispersant with the pigment particles and the print medium substrate. These dispersants have the general structure [PBP]ₙ—HL—[PMBP]ₙ where n ranges from 1 to about 5, more preferably n is 1 to about 3.

These triblock dispersants may be prepared using the chemical methods described above for the preparation of the diblock dispersants. The chemical methods are more suitable for triblock dispersants that contain a synthetic organic polymer as the hydrophilic linker. For triblock dispersants that contain a peptide as the hydrophilic linker, the chemical synthesis is complicated because it is difficult to control where the various blocks are attached to each other due to the large number of functional groups on the peptides. Therefore, the preferred method to prepare these totally peptide-based triblock dispersants is using the recombinant DNA and molecular cloning techniques described supra.

Applications of Dispersants

One application of the dispersants of the present invention is in aqueous inks, such as ink jet inks. Aqueous ink formulations are well known in the art. For example suitable formulations are described by Ma et al. in U.S. Pat. No. 5,272,201 and by Ma et al. in U.S. Pat. No. 5,085,698, both of which are incorporated herein by reference. Aqueous ink formulations typically comprise an aqueous carrier medium, a pigment or a mixture of pigments, a dispersant, and various other ingredients.

The aqueous carrier medium comprises water or a mixture of water and at least one water-soluble organic solvent. Deionized water is commonly used. Representative examples of water-soluble organic solvents are disclosed by Ma et al. in U.S. Pat. No. 5,085,698. The selection of a suitable mixture of water and water-soluble organic solvent depends upon the requirements of the specific application, such as the desired surface tension and viscosity, the selected pigment, drying time of the ink, and the type of media substrate onto which the ink will be printed. A mixture of a water-soluble polyhydric alcohol having at least 2 hydroxyl groups, e.g., diethylene glycol, and deionized water is preferred as the aqueous carrier medium, with water comprising between about 30% and about 95%, preferably about 60% to about 95%, by weight, based on the total weight of the aqueous carrier medium. The amount of aqueous carrier medium is in the range of about 70% to about 99.8%, preferably about 94% to about 99.8%, based on total weight of the ink when an organic pigment is selected, and about 25% to about 99.8%, preferably about 70 to about 99.8% when an inorganic pigment is selected.

The pigment may be a single pigment or a mixture of pigments. Suitable pigments are described by Ma et al. in U.S. Pat. No. 5,085,698. The ink may contain up to about 30% pigment by weight, preferably the amount of pigment is between about 0.1% to about 15% by weight.

Any of the dispersants of the present invention may be used alone or in combination in the ink formulation. It should also be noted that the pigment-binding peptides themselves, i.e., without a hydrophilic linker, may serve as aqueous dispersants under some conditions because of their hydrophilic nature. The dispersant is present in the ink in the range of about 0.1% to about 30% by weight.

Consistent with the requirements for the particular application, various types of aqueous additives can be used to modify the properties of the ink composition. Surfactant compounds may be used in addition to the dispersants of the present invention. These may be anionic, cationic, nonionic, or amphoteric surfactants. It is known in the art that certain surfactants may be incompatible with certain ink compositions and may destabilize the pigment dispersion. The choice of a specific surfactant is also highly dependent on the type of print medium substrate to be printed. It is expected that one skilled in the art can select the appropriate surfactant for the specific substrate to be used in the particular ink composition. In aqueous inks, the surfactants may be present in the amount of about 0.01% to about 5% and preferably about 0.2% to about 2%, based on the total weight of the ink. Co-solvents to improve penetration and pluggage inhibition properties of the ink composition may also be added, and in fact are preferred. Such co-solvents are well known in the prior art. Additionally, biocides may be used in the ink compositions to inhibit growth of microorganisms. Sequestering agents such as ethylenediaminetetraacetic acid (EDTA) may also be included to eliminate deleterious effects of heavy metal impurities. Other known additives, such as humectants, viscosity modifiers and other acrylic or non-acrylic polymers may also be added to improve various properties of the ink compositions as desired.

The ink compositions of the present invention are prepared in the same manner as other aqueous ink compositions, such as described by Ma et al. in U.S. Pat. No. 5,272,201.

The dispersants of the present invention may also be used as pigment dispersants in compositions for use in water-borne coating applications such as paints and color films. The coating compositions of the present invention may be applied to metal, ceramic, plastic, paper or wood surfaces. One particular application is in water-borne automotive coating compositions. Formulations for pigmented, water-borne coating compositions are well known in the art. For example, suitable formulations are described by Carpenter in U.S. Pat. No. 5,320,673, Barsotti in U.S. Pat. No. 5,376,704, and Goebel et al. in U.S. Pat. No. 6,350,809, all of which are incorporated herein by reference. Pigmented, water-borne coating compositions typically comprise an aqueous carrier medium, a pigment or a mixture of pigments, a dispersant, one or more film-forming resins, and various other ingredients.

The aqueous carrier medium comprises a mixture of water and one or more water-soluble organic solvents. Suitable water-soluble organic solvents include, but are not limited to, mono or polyhydric alcohols, glycol ethers or esters, glycols, and ketones. Typically 60 to 70% of the composition is water.

Suitable film-forming resins or binders are water dispersible or water-soluble ionic or nonionic resins. The resins may be acrylic, vinyl, polyurethane, polyester, alkyd, epoxy, or other polymers known to be useful in films. Examples of water-dispersible polymers used in water-borne coating compositions are described by Savino et al. in U.S. Pat. No. 4,794,147, Salatin et al. U.S. Pat. No. 4,791,168, and Kuwajima et al. U.S. Pat. No. 4,5,518,724, all of which are incorporated herein by reference.

The pigment may be a single pigment, or a mixture of pigments. Suitable pigments are described by Badejo et al. in U.S. Pat. No. 6,066,203, incorporated herein by reference, and by Goebel et al. supra. Organic pigments are used in the amount of about 1% to about 200% based on the total weight of the solid reactants, which includes the film forming resins and crosslinkers, and any other compounds that react and are incorporated into the polymer network during curing of the coating.

Any of the PBP—HL, PBP—PMBP or PMBP—HPL diblock dispersants or the PBP—HL—PMBP triblock dispersants of the present invention may be used alone or in combination in the coating compositions. The water-borne coating compositions of the present invention may also contain other ingredients that are well known in the art, including, but not limited to crosslinkers, plasticizers, additional cosolvents to aid in stabilization or application of the composition, rheology control agents, UV light stabilizers, antioxidants, catalysts, fungicides, and the like.

The dispersants of the present invention may also be used as pigment dispersants in compositions for pigmented plastics. The preparation and processing of plastics is well known in the art (see for example Ullman's Encyclopedia of Industrial Chemistry, 6^{th} edition, Vol. 27, Wiley-VCH, Hoboken, N.J. (2003), and Harper, Handbook of Plastic, Elastomers, and Composites, 3^{rd} edition, McGraw-Hill, New York (1996)). The use of pigment dispersants in plastics is well known in the art. Examples are described by May et al. in U.S. Pat. No. 5,652,316, Dietz et al. in U.S. Pat. No. 5,264,032, and Delphin et al. in U.S. Pat. No. 4,948,546, all of which are incorporated herein by reference.

The dispersants of the present invention may be used as pigment dispersants for a broad spectrum of standard plastic resins, including but not limited to, polymethyl methacrylate, polystyrene, poly-α-methylstyrene, and copolymers thereof, styrene-acrylonitrile copolymers, acrylonitrile-butadiene-styrene terpolymers, polycarbonates, polyethylene, polypropylene, polyacrylonitrile, polyvinyl chloride, chlorinated rubber, and polyvinylidene fluoride. A single pigment or a mixture of pigments may be used in the plastic compositions. Suitable pigments include the pigments described above and those described by May et al. supra. Typically, the pigments are used in an amount of about 0.5% to about 70%, preferably about 1% to about 50% by weight relative to the total weight of the composition.

Any of the PBP—HL, PBP—PMBP or PMBP—HPL diblock dispersants or the PBP—HL—PMBP triblock dispersants of the present invention may be used alone or in combination in the plastic compositions. The plastics may also contain other customary additives such as surfactants, rheological additives, preservatives, light stabilizers, and the like.

In yet another application, the cellulose-binding peptides of the present invention, specifically, SEQ ID NOs:35-39, may be used alone or in combination, as affinity tags for the recovery and purification of recombinant proteins. Any of these specific cellulose-binding peptide sequences, or combinations thereof, may be fused to the desired recombinant protein using the recombinant DNA and molecular cloning techniques described supra. If desired, the fusion proteins may be designed to contain sites for the proteolytic removal of the cellulose binding peptide from the heterologous protein. In this application, the cellulose-binding peptides are used in place of CBDs, the use of which as affinity tags is described by Tomme, et al. (Ann. N.Y. Acad. Sci. 799:418-424 (1996) and J. Chromatogr. B, 715:283-296 (1998)). The cellulose-binding peptides of the present invention have the advantage over CBDs of being smaller in size and therefore are easier to incorporate into recombinant proteins.

The affinity-tagged recombinant protein may be separated and purified by contacting the solution containing the protein with a cellulose support. Cellulose is commercially available in many different forms including beads, powders, fibers, membranes, filters, and sheets from a number of suppliers, including Sigma Chemical Co. (St. Louis, Mo.). The recombinant protein-containing solution may be contacted with the cellulose support in various ways known in the art. Examples include: contact in a batch reactor, filtration through a membrane, or passage through a cellulose-packed column. The protein solution is then separated from the cellulose support and the support is washed with a suitable buffer to remove unbound materials. Any buffer compatible with the protein may be used, including, but not limited to phosphate and Tris buffers of near neutral pH. The affinity-tagged protein is then recovered from the cellulose support by contacting it with an eluting agent. The eluting agent may be distilled water, a high pH solution (pH>10), a low pH solution (pH<3), a high concentration of guanidinium hydrochloride or urea, or with ethylene glycol. The preferred eluting agent is 0.2 M glycine-HCl at pH 2.2.

Similarly, the pigment-binding peptides of the invention, specifically SEQ ID NOs:5-10, 14-21,and 23-29, may be used alone or in combination, as affinity tags for the recovery and purification of recombinant proteins, as described above for the cellulose-binding peptides. The pigment-binding peptide sequences may be fused to the desired recombinant protein using the recombinant DNA and molecular cloning techniques described supra. The affinity-tagged recombinant protein may be separated and purified by contacting the solution containing the protein with the appropriate pigment in various ways known in the art, including but not limited to a batch reactor or passage through a pigment-packed column. The protein solution is then separated from the pigment and the pigment is washed with a suitable buffer to remove unbound materials, as described supra. The affinity-tagged protein is then optionally recovered from the pigment by contacting it with an eluting agent. Any of the eluting agents described above may be used.

Additionally, the cellulose-binding peptides of the present invention may also be used to immobilize proteins onto cellulose supports for biocatalytic and affinity applications. For these applications, the cellulose-binding peptides may be fused to the desired recombinant protein, as described above, or the binding peptides may be covalently coupled to native proteins using the peptide coupling methods described above. To immobilize the cellulose-binding peptide—protein conjugate, a solution containing the conjugate is simply contacted with the desired cellulose support.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmol” means micromole(s), “nmol” means nanomole(s), “g” means gram(s), “μg” means microgram(s), “mg” means milligram(s), “g” means the gravitation constant, “J” means joules, “mJ” means millijoules”, “v/v” means volume to volume ratio, “pfu(s) means plaque-forming unit(s), “kDA” means kilodalton(s), “BSA” means bovine serum albumin, “ELISA” means enzyme linked immunosorbent assay, “IPTG” means isopropyl β-D-thiogalactopyranoside, “OD” means optical density, “OD₄₀₅” means the optical density measured at a wavelength of 405 nm, “OD₆₀₀” means the optical density measured at a wavelength of 600 nm”, “P” means pressure, “P₀” means the vapor pressure of the liquid adsorbate at the temperature of the isotherm, “TBS” means Tris-buffered saline, “TBST-X” means Tris-buffered saline containing a designated amount (X %) of Tween® 20, “HRP” means horseradish peroxidase, “Xgal” means 5-bromo-4-chloro-3-indolyl-beta-D-galactopyranoside, “DMF” means dimethylformamide, “DP” means degree of polymerization, “THF” means tetrahydrofuran, “NMR” means nuclear magnetic resonance spectroscopy, “TBP” means triblock protein, “SDS-PAGE” means sodium dodecyl sulfate-polyacrylamide gel electrophoresis, and “MALDI” means matrix assisted laser desorption ionization mass spectrometry.

General Methods:

Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, (1989) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987).

Materials and methods suitable for the maintenance and growth of bacterial cultures are well known in the art. Techniques suitable for use in the following Examples may be found as set out in Manual of Methods for General Bacteriology (Philipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989). All reagents, restriction enzymes and materials used for the growth and maintenance of bacterial cells were obtained from Aldrich Chemicals (Milwaukee, Wis.), BD Diagnostic Systems (Sparks, Md.), Life Technologies (Rockville, Md.), New England BioLabs (Beverly, Mass.), Promega (Madison, Wis.) or Sigma Chemical Company (St. Louis, Mo.), unless otherwise specified.

Phage Display Peptide Libraries

The phage display libraries used in the present invention, Ph.D.-7 and Ph.D.-12, were purchased from New England BioLabs (Beverly, Mass.). These libraries are based on combinatorial libraries of random peptide 7- and 12-mers fused to a minor coat protein (plII) of M13 Phage. The displayed peptides are expressed at the N-terminus of plII and linked with it through a short spacer of GGGS. The Ph.D.-7 and Ph.D.-12 libraries consist of approximately 2.8×10⁹ and 2.7×10⁹ sequences, respectively.

Screening of Peptide Display Libraries

A standard procedure provided by the supplier, New England BioLabs, was modified to fit our experimental needs. A general description of this method is as follows.

Biopanning: An appropriate amount of the substrate to be tested was placed in a microcentrifuge tube or a well of a 24-well plate. The tube or well was then filled with block buffer containing 5 mg/mL BSA and 0.1 M NaHCO₃ at pH 8.6, and the substrate was incubated for 1 h at 4° C. The substrate and container were washed 6 times with TBST-0.1% (50 mM Tris-HCl, pH 7.5, and 150 mM NaCl (TBS) containing 0.1% Tween® 20). The substrate was then incubated with 1 mL of TBST-0.1% containing 2×10¹¹ phage (10 mL of original library) and 1 mg/mL BSA for 10-20 min at room temperature. After this time, the substrate was washed 5 times with TBST-X (TBS with a designated amount of Tween® 20, as given infra for the specific experiments) containing 1 mg/mL BSA and then washed an additional 5 times with TBST-X alone. Substrate-binding phage was eluted by incubating the substrate with 1 mL of elution solution, containing 1 mg/mL BSA and 0.2 M Glycine-HCl at pH 2.2, for 7 min. The elution solution was neutralized immediately by adding 150 μL of 1 M Tris-HCl, pH 9.1.

Titration and Sequencing

The eluted phage was diluted with LB (Luria-Bertani) medium, consisting of 1% Bacto-tryptone, 0.5% Bacto-yeast extract, and 1% NaCl adjusted to pH 7.5 with NaOH, to prepare 10-fold serial dilutions of 10¹ to 10⁴. A 10 μL aliquot of each dilution was incubated with 200 μL of mid-log phase E. coli ER2738 (New England BioLabs), grown in LB medium, for 2 min and then mixed with 3 mL of agarose top (LB medium with 5 mM MgCl₂, and 0.7% agarose) at 45° C. This mixture was spread onto an LB medium/IPTG/Xgal plate (LB medium with 15 g/L agar, 0.05 g/L IPTG, and 0.04 g/L Xgal) and incubated overnight at 37° C. The blue plaques were counted to calculate the phage titer.

To determine fusion peptide sequences in the substrate-binding phage, a number of blue plaques were randomly picked from the plate and incubated with 2 mL of a 100-fold LB dilution of an overnight culture of E. coli ER2738 for 5 h in a 37° C. shaker. Phage DNA was purified from the culture using a QIAprep Spin M13 Kit (QIAGEN, Valencia, Calif.) and sequenced in the DuPont sequencing facility by utilizing—96 gIII primer, SEQ ID NO:41 (5′CCCTCATAGTTAGCGTAAGG3′). The DNA sequence upstream of the coding region of the GGGS spacer (21 nucleotides for a Ph.D.-7 phage or 36 nucleotides for a Ph.D.-12 phage) was translated into a peptide sequence.

Amplification

To identify candidate peptide sequences, several cycles of the panning process were usually required under different conditions. For this purpose, half of the eluted phage from the previous run was amplified by incubating with 20 mL of a 100-fold LB dilution of the overnight culture of E. coli ER2738 for 4.5 h in a 37° C. shaker. Bacteria were removed by centrifugation at 10,000×g. Phage was precipitated by adding 1/6 volume of PEG/NaCl (20% PEG-8,000, 2.5 M NaCl) and collected by centrifugation at 10,000×g. Phage was suspended in 1 mL of TBS and subjected to PEG precipitation again. The amplified phage was re-suspended in 0.2 mL TBS and its titer was determined by following the protocol described supra. The phage was then ready for the next run of panning. In these experiments, the only difference between runs was the concentration of Tween® 20 that was applied in the TBST-X during washing.

### Example 1

### Identification of Pigment-Binding Peptides

The purpose of this Example was to identify peptide sequences that specifically bind to common ink pigments using phage display screening.

Four commercially available ink pigments were used as test substrates in this study. These pigments included Carbon Black FW-18 (Degussa, Piscataway, N.J.), Cromophtal® Yellow 131AK (Ciba Specialty Chemicals, High Point, N.C.), Sunfast® Magenta 122 (Sun Chemical, Carstadt, N.J.), and Sunfast® Blue 15:3 (Sun Chemical). Both the Ph.D.-7 and the Ph.D.-12 screening libraries were used to identify pigment-binding candidates.

The screening of the peptide display libraries was done as described supra with the following modifications. A 250 μg sample of Carbon Black FW-18 was used as the test substrate in a microcentrifuge tube. Three runs of screenings were conducted with 0.1%, 0.5%, and 1% Tween® 20 in TBST-X, respectively, followed by two parallel fourth runs with 1% and 2% Tween® 20 in TBST-X. For screening with the Ph.D.-12 library, only one fourth run was conducted with 2% Tween® 20 in TBST-X.

This screening was repeated using the Cromophtal® Yellow 131AK, Sunfast® Magenta 122, and Sunfast® Blue 15:3 pigments as the test substrate. In these experiments, 500 μg of the pigments was used.

To determine the peptide sequences, DNA from 16 to 24 phage plaques was sequenced in each run, as described supra. Based on these results, sequences with high reappearance frequency in a particular run and/or sequences which consistently interacted with pigments in several runs under various stringency conditions were identified as pigment-binding peptide candidates. These sequences are given in Table 1.

TABLE 1

Pigment-Binding Peptide Candidates

| | | | |
|---|---|---|---|
| | Designated | Peptide | SEQ ID |
| Pigment | Name | Sequence | NO: |

| | | | |
|---|---|---|---|
| Carbon Black | CB-71 | MPPPLMQ | 7 |
| | CB-72 | FHENWPS | 6 |
| | CB-121 | RTAPTTPLLLSL | 8 |
| | CB-122 | WHLSWSPVPLPT | 9 |
| Cromophtal ® Yellow | CY-71 | PHARLVG | 10 |
| | CY-72 | NIPYHHP | 11 |
| | CY-73 | TTMPAIP | 12 |
| | CY-74 | HNLPPRS | 13 |
| | CY-121 | AHKTQMGVRQPA | 14 |
| | CY-122* | ADNVQMGVSHTP | 15 |
| | CY-123* | AHNAQMGVSHPP | 16 |
| | CY-124* | ADYVGMGVSHRP | 17 |
| | CY-125 | SVSVGMKPSPRP | 5 |
| Sunfast ® Magenta | SM-71 | YPNTALV | 18 |
| | SM-72 | VATRIVS | 19 |
| | SM-121 | HSLKNSMLTVMA | 20 |
| Sunfast ® Blue | SB-71 | NYPTQAP | 21 |
| | SB-72 | KCCYSVG | 22 |
| | SB-121 | RHDLNTWLPPVK | 23 |
| | SB-122 | EISLPAKLPSAS | 24 |
| | SB-123 | SVSVGMKPSPRP | 5 |
| | SB-124** | SDYVGMRPSPRH | 25 |
| | SB-125** | SDYVGMRLSPSQ | 26 |
| | SB-126** | SVSVGIQPSPRP | 27 |
| | SB-127** | YVSVGIKPSPRP | 28 |
| | SB-128** | YVCEGIHPCPRP | 29 |

*These sequences are analogs of CY-121.
**These sequences are either analogs of SB-123 or are similar to the analogs of SB-123.

### Example2

### Identification of Print Medium-Binding Peptides

The purpose of this Example was to identify peptide sequences that specifically bind to common print media using phage display screening.

The print media tested as substrates in this study included: 100% cotton fabric (Cotton Broadcloth Style 419W obtained from Testfabics, West Pittston, Pa.), 65/35 polyester/cotton fabric (PrintCloth (65/35PC) 7436M obtained from Testfabrics), and Hammermill® Tidal MP paper (First State Paper, Wilmington, Del.). A piece of the print medium (7.5 mm×7.5 mm) was used in Ph.D.-7 library screening to identify print medium-binding peptide candidates using the procedure described supra. Screening was carried out in a 24-well plate, and the binding phage was eluted into a different well than that used for the binding. Three runs of screenings were conducted with 0.5% Tween® 20 in TBST-X, followed by four additional runs with 1%, 3%, 3%, and 5% Tween® 20 in TBST-X, respectively. To determine the peptide sequences, DNA from 16 phage plaques was sequenced in each run, as described supra.

In these studies, a high background due to non-specific interactions between the phage and all three print media was observed in all runs, including the 6^{th} run with 5% Tween® 20 in TBS-X. This background prevented the enrichment of phage that specifically interacted with the print media, thus interfering with the screening. However, a careful inspection of the sequence data indicated that a few of the sequences in each of the three screenings showed a consistent interaction with the substrate being tested and showed some degree of enrichment. These sequences, which are listed in Table 2, were identified as print medium-binding peptide candidates.

TABLE 2

Print Medium-Binding Peptide Candidates

| | | | |
|---|---|---|---|
| | Designated | Peptide | SEQ ID |
| Print Medium | Name | Sequence | NO: |

| | | | |
|---|---|---|---|
| Cotton fabric | COT-71* | SILPYPY | 30 |
| | COT-72 | STASYTR | 31 |
| Polyester/cotton fabric | P/C-71 | LPVRPWT | 32 |
| | P/C-72* | SILPYPY | 30 |
| Hammermill ® paper | HCP-71 | GNTPSRA | 33 |
| | HCP-72 | HAIYPRH | 4 |
| | HCP-73 | YQDSAKT | 34 |
| | HCP-74* | SILPYPY | 30 |

*These sequences are identical.

### Example 3

Identification of Cellulose and Poly(Ethylene Terephthalate)-Binding Peptide Candidates

The purpose of this Example was to identify peptide sequences that specifically bind to cellulose and poly(ethylene terephthalate) using phage display screening. Because of the difficulty encountered in clearly identifying print medium-binding peptides due to high background, additional studies were done to identify peptides that specifically interact with cellulose and poly(ethylene terephthalate), two major ingredients of those print media.

In this study, 250 μg of long fibrous cellulose with a fiber size from 100 to 400 μm (Sigma Chemical Company, St Louis, Mo.) was used in Ph.D.-7 and Ph.D.-12 library screenings. Reactions were carried out in microcentrifuge tubes following the procedure described in Example 1. Five runs was done with 0.1%, 0.5%, 1%, 2%, and 4% Tween® 20 in TBST-X for Ph.D-7 library screening, but only the first four runs were done for Ph.D.-12 library screening.

A poly(ethylene terephthalate) pellet (DuPont Co., Wilmington, Del.) with a size of approximately 2 mm×2 mm×1 mm, was also used in Ph.D.-12 library screening, following the same procedure described in Example 2. Two runs were conducted with 0.1% and 0.5% Tween® 20 in TBST-X, followed by parallel third runs with 0.5% and 1% Tween® 20 in TBST-X.

The results from this study showed that a few sequences in each of the three screenings satisfied the criteria of a substrate-binding peptide; specifically, a high reappearance frequency in a particular run and/or a consistent interaction with the substrate in several runs under various stringency conditions. These sequences, which are listed in Table 3, were identified as cellulose or poly(ethylene terephthalate)-binding peptide candidates.

TABLE 3

Cellulose and Poly(ethylene terephthalate)-
Binding Peptide Candidates

| | | | |
|---|---|---|---|
| Print Medium | Designated | Peptide | SEQ ID |
| Ingredient | Name | Sequence | NO: |

| | | | |
|---|---|---|---|
| Cellulose | CEL-71 | VPRVTSI | 35 |
| | CEL-72 | MANHNLS | 36 |
| | CEL-73 | FHENWPS | 6 |
| | CEL-121 | THKTSTQRLLAA | 37 |
| | CEL-122 | KCCYVNVGSVFS | 38 |
| | CEL-123 | AHMQFRTSLTPH | 39 |
| Poly(ethylene | PET-121 | GTSDHMIMPFFN | 40 |
| terephthalate) | | | |

### Example 4

Confirmation of Substrate-Binding Peptides by ELISA-Based Binding Assay

The purpose of this Example was to confirm the substrate-binding peptide candidates that were identified by phage display screening using an ELISA-based binding assay.

Some peptides may be selected from a screening process for reasons other than substrate-specific binding activity, for example, a special phage's fast growth rate during amplification. To confirm its substrate-specific binding activity, phage that harbored a peptide candidate was amplified and subjected to an ELISA-based binding assay. Two phages obtained from the Ph.D.-7 and Ph.D.-12 libraries served as controls for the 7- and 12-amino acid-peptide candidates in the assay, respectively. One phage had the 7-amino acid-peptide sequence SNRDLVY, designated Ctrl-71 (SEQ ID NO:42). The other control phage had the 12-amino acid peptide sequence SSNLNLSWVQDT, designated Ctrl-121 (SEQ ID NO:43). In some binding assays, a third phage, designated as Ctrl-122, containing the 12-amino acid peptide KFQNMDRHPASL (SEQ ID NO:44) was used as a control.

To perform the assay, phages that harbor the candidate peptides were purified and amplified, as described supra, and 8×10¹⁰ copies of the purified phages were used. Assays were done for the pigment-binding peptides, the print medium-binding peptides, and the print medium ingredient-binding peptides. In the pigment-binding assay, 75 μg carbon black, 150 μg Cromophtal® Yellow, 150 μg Sunfast® Magenta, or 150 μg Sunfast® Blue was used as the substrate. In the print medium-binding assay, individual strings of fiber were pulled from 100% cotton 419W fabric and 65/35 polyester/cotton 7436M fabric. The 20-mm segments of those fibers, or 16 mm² Hammermill Tidal MP paper were used in the assay. In the ingredient-binding assay, 150 μg Type 20 Sigmacell Cellulose (Sigma Chemical Co.) or 450 μg poly(ethylene terephalate) powder, prepared by grinding the pellets, described in Example 3, in a SPEX 6700 Freezer Mill (SPEX CertiPrep Inc, Metuchen, N.J.), was used.

The ELISA-based- assay was done in a MultiScreen-HV (0.45 μm) 96 well filtration plate obtained from Millipore Corp. (Bedford, Mass.). A vacuum manifold (Millipore corp.) was used to empty the wells in the assay by evacuation of the solutions through the membrane.

The assay was performed as follows. The appropriate amount of the substrate to be tested, as described supra, was placed in the well of the 96 well filtration plate. The substrates and the wells were blocked by adding 200 μL of block buffer, consisting of 5 mg/mL BSA, 0.1 M NaHCO₃ at pH 8.6, into each well and shaking for 1 h at room temperature using an Orbimix 110 shaker (Brinkmann Instruments Inc., Westbury, N.Y.). The block buffer was removed from the wells using vacuum and the substrates were washed five times using 200 μL portions of TBST-0.1% (TBS+0.1% Tween® 20). The appropriate amount of phage (8×10¹⁰ copies) in 200 μL TBST-0.1% containing 1 mg/mL BSA was added to each well. The plate was shaken at room temperature for 15 min. The phage solution was removed using vacuum and the substrates were washed four times using 200 μL portions of TBST-0.5% containing 1 mg/mL BSA, and four more times using 200 μL portions of TBST-0.5%. A HRP/anti-M13 monoclonal antibody conjugate, obtained from Amersham Biosciences (Piscataway, N.J.), was diluted 5000-fold with TBS containing 1 mg/mL BSA and added to each well. The plate was shaken for 1 h at room temperature. After this time, the conjugate solution was removed using vacuum and the substrates were washed five times with TBST-0.1%. Then, 200 μL of ABTS Substrate Solution (0.4 mM 2,2′-azino-bis(3-ethylbenzthiazoline-6-sulfonic acid, 0.05% H₂O₂, and 50 mM sodium citrate at pH 4.0) was added to each well and the plate was shaken at room temperature for 20 min. The resulting colored solutions were transferred to a normal 96-well plate using the vacuum manifold and the OD at 405 nm of the solutions was measured using a kinetic microplate reader, obtained from Molecular Devices (Menlo Park, Calif.).

Each peptide was run in triplicate in the assay and the average OD₄₀₅ values and the standard deviations are summarized in Tables 4-6.

TABLE 4

Results of ELISA Binding Assays with Pigments

| | | | |
|---|---|---|---|
| | | SEQ ID | |
| Pigment | Peptide | NO: | OD₄₀₅ |

| | | | |
|---|---|---|---|
| Carbon Black | CB-71 | 7 | 2.20 ± 0.12 |
| | CB-72 | 6 | 3.63 ± 0.05 |
| | Ctrl-71 | 42 | 0.08 ± 0.03 |
| | CB-121 | 8 | 0.55 ± 0.03 |
| | CB-122 | 9 | 2.42 ± 0.24 |
| | Ctrl-121 | 43 | 0.11 ± 0.01 |
| Cromophtal ® | CY-71 | 10 | 2.39 ± 0.16 |
| Yellow | CY-72 | 11 | 0.23 ± 0.08 |
| | CY-73 | 12 | 0.12 ± 0.03 |
| | CY-74 | 13 | 0.10 ± 0.06 |
| | Ctrl-71 | 42 | 0.09 ± 0.01 |
| | CY-121 | 14 | 0.53 ± 0.03 |
| | CY-122 | 15 | 0.60 ± 0.05 |
| | CY-123 | 16 | 0.45 ± 0.04 |
| | CY-124 | 17 | 0.40 ± 0.05 |
| | CY-125 | 5 | 0.54 ± 0.05 |
| | Ctrl-121 | 43 | 0.08 ± 0.02 |
| Sunfast ® | SM-71 | 18 | 0.86 ± 0.05 |
| Magenta | SM-72 | 19 | 0.43 ± 0.18 |
| | Ctrl-71 | 42 | 0.08 ± 0.06 |
| | SM-121 | 20 | 2.17 ± 0.17 |
| | Ctrl-121 | 43 | 0.22 ± 0.03 |
| Sunfast ® Blue | SB-71 | 21 | 0.75 ± 0.03 |
| | SB-72 | 22 | 0.02 ± 0.02 |
| | Ctrl-71 | 42 | 0.15 ± 0.02 |
| | SB-121 | 23 | 0.21 ± 0.02 |
| | SB-122 | 24 | 0.33 ± 0.03 |
| | SB-123 | 5 | 0.55 ± 0.09 |
| | SB-124 | 25 | 0.62 ± 0.06 |
| | SB-125 | 26 | 0.58 ± 0.07 |
| | SB-126 | 27 | 0.34 ± 0.06 |
| | SB-127 | 28 | 0.65 ± 0.06 |
| | SB-128 | 29 | 0.78 ± 0.11 |
| | Ctrl-121 | 43 | 0.11 ± 0.00 |

TABLE 5

Results of ELISA Binding Assays with Print
Media

| | | | |
|---|---|---|---|
| | | SEQ ID | |
| Print Medium | Peptide | NO: | OD₄₀₅ |

| | | | |
|---|---|---|---|
| Cotton Fabric | COT-71 | 30 | 0.39 ± 0.02 |
| | COT-72 | 31 | 0.37 ± 0.05 |
| | Ctrl-71 | 42 | 0.16 ± 0.03 |
| Polyester/Cotton | P/C-71 | 32 | 0.33 ± 0.02 |
| Fabric | P/C-72 | 30 | 0.37 ± 0.05 |
| | Ctrl-71 | 42 | 0.17 ± 0.03 |
| Hammermill | HCP-71 | 33 | 0.18 ± 0.02 |
| Paper | HCP-72 | 4 | 0.22 ± 0.06 |
| | HCP-73 | 34 | 0.31 ± 0.05 |
| | HCP-74 | 30 | 0.26 ± 0.03 |
| | Ctrl-71 | 42 | 0.08 ± 0.01 |

TABLE 6

Results of ELISA Binding Assays with Print
Media Ingredients

| | | | |
|---|---|---|---|
| | | SEQ ID | |
| Ingredient | Peptide | NO: | OD₄₀₅ |

| | | | |
|---|---|---|---|
| Sigmacell | CEL-71 | 35 | 0.16 ± 0.03 |
| Cellulose | CEL-72 | 36 | 0.22 ± 0.01 |
| | CEL-73 | 6 | 0.07 ± 0.01 |
| | Ctrl-71 | 42 | 0.06 ± 0.01 |
| | CEL-121 | 37 | 0.41 ± 0.02 |
| | CEL-122 | 38 | 0.30 ± 0.01 |
| | CEL-123 | 39 | 0.09 ± 0.01 |
| | Ctrl-121 | 43 | 0.10 ± 0.01 |
| Polyester | PET-121 | 40 | 0.27 ± 0.02 |
| Powder | Ctrl-122 | 44 | 0.10 ± 0.01 |

These results confirm that the majority of the peptide candidates identified from the screening process were indeed specific substrate-binding peptides because they had OD₄₀₅ values that were significantly higher than those of the controls. The following peptides CY-72, SB-72, CEL-73, CY-73, CY-74, and CEL-123 did not have OD₄₀₅ values that were significantly higher than the controls, indicating that they are false positive binding peptides. In general, these results show that the pigment-binding peptides have a higher binding affinity for their respective substrates than the print medium and print medium ingredient-binding peptides, although the high surface area of the pigment particles relative to the print media substrates may contribute to the higher OD₄₀₅ values.

### Example 5

### Confirmation of Substrate-Binding Peptides by Direct Phage Counting

The purpose of this Example was to confirm the results of the ELISA-binding assay using direct phage counting.

In order to confirm the correlation between the OD₄₀₅ values obtained in Example 4 and the actual binding affinity of the peptides for their respective substrates, the peptides with the highest OD₄₀₅ values in each group were tested using a direct phage counting procedure as follows. The selected peptides were interacted with their specific substrate in a 96-well filtration plate, as described in Example 4. The procedure described in Example 4 was followed except that instead of adding the HRP/anti-M13 monoclonal antibody conjugate, the bound phages were eluted by adding 200 μL of elution solution (0.2 M glycine-HCl, 1 mg/mL BSA at pH 2.2) to each well and mixing at room temperature for 5 min. The phage-containing solutions were transferred to a regular 96-well plate using a vacuum manifold and were immediately neutralized by adding 30 μL 1 M Tris-HCl at pH 9.1. The number of phages that were bound to their substrate in the assay was determined by phage titration, as described supra.

The average number of bound phages from three independent assays and the standard deviation of the results are given in Tables 7-9.

TABLE 7

Results of Phage Counting Assays with Pigments

| | | | | |
|---|---|---|---|---|
| | | | Phage | |
| | | SEQ ID | Titer | Standard |
| Pigment | Peptide | NO. | (Pfu/10 μL) | Deviation |

| | | | | |
|---|---|---|---|---|
| Carbon Black | CB-72 | 6 | 3.0 × 10⁵ | 3.9 × 10⁴ |
| | Ctrl-71 | 42 | 2.4 × 10³ | 2.9 × 10² |
| | CB-122 | 9 | 6.5 × 10⁵ | 3.9 × 10⁴ |
| | Ctrl-121 | 43 | 2.6 × 10³ | 2.9 × 10² |
| Cromophtal ® | CY-71 | 10 | 4.6 × 10⁵ | 4.0 × 10⁴ |
| Yellow | Ctrl-71 | 42 | 5.3 × 10³ | 6.8 × 10² |
| | CY-121 | 14 | 8.7 × 10⁵ | 4.5 × 10⁴ |
| | Ctrl-121 | 43 | 1.1 × 10³ | 2.4 × 10² |
| Sunfast ® | SM-71 | 18 | 8.8 × 10³ | 1.6 × 10² |
| Magenta | Ctrl-71 | 42 | 1.5 × 10³ | 2.0 × 10² |
| | SM-121 | 20 | 1.3 × 10⁴ | 8.4 × 10² |
| | Ctrl-121 | 43 | 2.4 × 10³ | 2.7 × 10² |
| Sunfast ® Blue | SB-71 | 21 | 8.5 × 10⁵ | 7.5 × 10⁴ |
| | Ctrl-71 | 42 | 1.6 × 10³ | 2.3 × 10² |
| | SB-128 | 29 | 3.0 × 10⁴ | 2.1 × 10³ |
| | Ctrl-121 | 43 | 2.1 × 10³ | 1.1 × 10² |

TABLE 8

Results of Phage Counting Assays with Print
Media

| | | | Phage | |
|---|---|---|---|---|
| | | SEQ ID | Titer | Standard |
| Print Medium | Peptide | NO. | (Pfu/10 μL) | Deviation |
| Cotton Fabric | COT-71 | 30 | 2.9 × 10³ | 1.1 × 10² |
| | Ctrl-71 | 42 | 1.1 × 10³ | 7.6 × 10¹ |
| Polyester/ | P/C-72 | 30 | 1.9 × 10³ | 8.6 × 10¹ |
| Cotton Fabric | Ctrl-71 | 42 | 5.3 × 10² | 4.6 × 10¹ |
| Hammermill | HCP-74 | 30 | 8.6 × 10³ | 6.4 × 10¹ |
| Paper | Ctrl-71 | 42 | 2.1 × 10³ | 1.2 × 10² |

TABLE 9

Results of Phage Counting Assays with Print
Media Ingredients

| | | | Phage | |
|---|---|---|---|---|
| | | SEQ ID | Titer | Standard |
| Ingredient | Peptide | NO. | (Pfu/10 μL) | Deviation |
| Sigmacell | CEL-71 | 35 | 8.9 × 10³ | 1.1 × 10³ |
| Cellulose | Ctrl-71 | 42 | 1.1 × 10³ | 1.1 × 10² |
| | CEL-121 | 37 | 4.9 × 10⁵ | 5.2 × 10⁴ |
| | Ctrl-121 | 43 | 8.9 × 10² | 1.2 × 10² |
| Polyester | PET-121 | 40 | 4.1 × 10³ | 2.8 × 10² |
| Powder | Ctrl-122 | 44 | 1.5 × 10³ | 5.9 × 10¹ |

These results demonstrate that most of the pigment-binding peptides examined have a high specific binding affinity, as shown by the fact that the number of phages that bind to the pigments is at least one order of magnitude higher than that of the controls. The exceptions are SM-71 and SM-121 for which the number of bound phages is about 5-fold higher than that of the control, indicating that these peptides have a moderate specific binding affinity. For the print medium and print medium ingredient-binding peptides, the number of bound phages was statistically higher than that of the controls, but was within the same order of magnitude, indicating a moderate specific binding affinity. One exception is CEL-121, which had a number of bound phages that was more than two orders of magnitude greater than that of the control. In addition to binding affinity, the surface area of the pigment, print medium, and print medium ingredients also contribute to the phage number that bind with those substrates. These results are consistent with those obtained with the ELISA-based assay results of Example 4 and confirm the specific substrate-binding peptide candidates.

### Example 6

### Characterization of the Binding Peptide Candidates by Measuring Their Binding Affinity (MB₅₀) and Maximum Binding (Bₘₐₓ)

In Examples 4 and 5, the substrate-specific binding activity of the phages that harbor the peptide candidates was confirmed using an ELISA assay and a direct plaque counting method. However, those methods have some limitations. Specifically, they do not indicate how strongly each candidate binds to its corresponding substrate and the phage itself may cause a false positive in the methods. To overcome these limitations, the binding peptide candidates were isolated from the phage and were fused to a detectable protein partner. Glutathione S-transferase (GST) was used as the protein partner. Through the titration of binding activity, the binding affinity (MB₅₀) and maximum binding (Bₘₐₓ) were measured for the selected peptide candidates.

Construction of pGSTf Plasmids:

The expression plasmid pGSTf was created by modifying the pDEST15 plasmid of the Gateway system (Invitrogen, Carlsbad, Calif.). The modification simplified the C-terminal sequence of GUS on the pDEST15 plasmid and added a linker coding sequence and four unique restriction sites (Ncol, Sacl, Notl, and Ascl) right before the Stop codon of the GUS gene. Because of these unique restriction sites, a double stranded DNA adaptor that contained a binding peptide coding sequence and the flanking Sacl and Ascl complementary sticky ends was inserted into the pGSTf plasmid between the Sacl and Ascl sites.

GST-peptide fusions were constructed for the two best 7-mer sequences and the two best 12-mer sequences from each group of binding peptide candidates. A pair of oligonucleotides was designed for each peptide candidate, as shown in Table 10. The oligonucleotides for each peptide were annealed by heating for 5 min at 95° C. and then cooling to 24° C. at a rate of 2° C. per minute. Finally, the annealed oligonucleotide was inserted into pGSTf between the Sacl and Ascl sites by employing routine molecular recombination techniques, forming the expression plasmid pGST-peptide. The oligonucleotides used for peptide CEL-121 had a different form than the oligonucleotides used for the other peptides. The oligonucleotide pair for CEL-121 was inserted into pGSTf between the Ncol and Ascl sites to form the plasmid pGST-CEL121.

TABLE 10

Oligonucleotides Used to Prepare pGSTf Plasmids

| | | | | |
|---|---|---|---|---|
| Peptide in | Oligonu- | SEQ ID | Oligonu- | SEQ ID |
| GST Fusion | cleotide 1 | NO: | cleotide 2 | NO: |

| | | | | |
|---|---|---|---|---|
| CB-122 | GGCCGCCTG | 67 | CGCGCCTTAA | 68 |
| | GCATTTGTCG | | GTAGGCAAAG | |
| | TGGTCTCCTG | | GAACAGGAGA | |
| | TTCCTTTGCC | | CCACGACAAA | |
| | TACTTAAGG | | TGCCAGGCG | |
| | | | GCCGC | |
| CB-121 | GGCCGCCCG | 69 | CGCGCCTTAA | 70 |
| | GACTGCGCCT | | AGCGACAGAA | |
| | ACTACGCCGC | | GAAGCGGCG | |
| | TTCTTCTGTC | | TAGTAGGCGC | |
| | GCTTTAAGG | | AGTCCGGGC | |
| | | | GGCCGC | |
| CB-72 | GGCCGCCTTT | 71 | CGCGCCTTAC | 72 |
| | CATGAGAATT | | GAAGGCCAAT | |
| | GGCCTTCGTA | | TCTCATGAAA | |
| | AGG | | GGCGGCCGC | |
| CB-71 | GGCCGCCAT | 73 | CGCGCCTTAC | 74 |
| | GCCTCCGCC | | TGCATCAACG | |
| | GTTGATGCAG | | GCGGAGGCA | |
| | TAAGG | | TGGCGGCCG | |
| | | | C | |
| SM-71 | GGCCGCCTAT | 75 | CGCGCCTTAC | 76 |
| | CCGAATACTG | | ACCAACGCAG | |
| | CGTTGGTGTA | | TATTCGGATA | |
| | AGG | | GGCGGCCGC | |
| SM-121 | GGCCGCCCA | 77 | CGCGCCTTAC | 78 |
| | TTCGCTTAAG | | GCCATCACAG | |
| | AATTCGATGC | | TAAGCATCGA | |
| | TTACTGTGAT | | ATTCTTAAGC | |
| | GGCGTAAGG | | GAATGGGCG | |
| | | | GCCGC | |
| SM-72 | GGCCGCCGT | 79 | CGCGCCTTAA | 80 |
| | TGCTACGAGG | | GACACAATCC | |
| | ATTGTGTCTT | | TCGTAGCAAC | |
| | AAGG | | GGCGGCCGC | |
| CY-72 | GGCCGCCAAT | 81 | CGCGCCTTAC | 82 |
| | ATTCCTTATC | | GGATGATGAT | |
| | ATCATCCGTA | | AAGGAATATT | |
| | AGG | | GGCGGCCGC | |
| CY-71 | GGCCGCCCC | 83 | CGCGCCTTAA | 84 |
| | GCATGCTCGT | | CCCACCAAAC | |
| | TTGGTGGGTT | | GAGCATGCG | |
| | AAGG | | GGGCGGCCG | |
| | | | C | |
| CY-121 | GGCCGCCGC | 85 | CGCGCCTTAA | 86 |
| | GCATAAGACG | | GCCGGCTGC | |
| | CAGATGGGTG | | CTCACACCCA | |
| | TGAGGCAGC | | TCTGCGTCTT | |
| | CGGCTTAAGG | | ATGCGCGGC | |
| | | | GGCCGC | |
| CY-125 | GGCCGCCTCT | 87 | CGCGCCTTAA | 88 |
| | GTTTCTGTGG | | GGCCTAGGA | |
| | GTATGAAGCC | | CTCGGCTTCA | |
| | GAGTCCTAGG | | TACCCACAGA | |
| | CCTTAAGG | | AACAGAGGC | |
| | | | GGCCGC | |
| SB-72 | GGCCGCCAA | 89 | CGCGCCTTAC | 90 |
| | GTGTTGTTAT | | CCCACAGAAT | |
| | TCTGTGGGGT | | AACAACACTT | |
| | AAGG | | GGCGGCCGC | |
| SB-71 | GGCCGCCAAT | 91 | CGCGCCTTAC | 92 |
| | TATCCTACGC | | GGAGCCTGC | |
| | AGGCTCCGTA | | GTAGGATAAT | |
| | AGG | | TGGCGGCCG | |
| | | | C | |
| SB-121 | GGCCGCCGA | 93 | CGCGCCTTAC | 94 |
| | GATTTCGCTT | | GAAGCACTAG | |
| | CCTGCTAAGC | | GCAGCTTAGC | |
| | TGCCTAGTGC | | AGGAAGCGAA | |
| | TTCGTAAGG | | ATCTCGGCGG | |
| | | | CCGC | |
| SB-128 | GGCCGCCTAT | 95 | CGCGCCTTAA | 96 |
| | GTGTGTGAGG | | GGCCTAGGA | |
| | GGATTCATCC | | CATGGATGAA | |
| | ATGTCCTAGG | | TCCCCTCACA | |
| | CCTTAAGG | | CACATAGGCG | |
| | | | GCCGC | |
| COT-71, | GGCCGCCTCT | 97 | CGCGCCTTAA | 98 |
| P/C-72, and | ATTCTGCCGT | | TAAGGATACG | |
| HCP-74 | ATCCTTATTAA | | GCAGAATAGA | |
| | GG | | GGCGGCCGC | |
| COT-72 | GGCCGCCTC | 99 | CGCGCCTTAA | 100 |
| | GACGGCGTCT | | CGAGTATAAG | |
| | TATACTCGTT | | ACGCCGTCGA | |
| | AAGG | | GGCGGCCGC | |
| P/C-71 | GGCCGCCCTT | 101 | CGCGCCTTAA | 102 |
| | CCGGTGCGT | | GTCCACGGAC | |
| | CCGTGGACTT | | GCACCGGAA | |
| | AAGG | | GGGCGGCCG | |
| | | | C | |
| HCP-73 | GGCCGCCTAT | 103 | CGCGCCTTAC | 104 |
| | CAGGATTCTG | | GTTTTCGCAG | |
| | CGAAAACGTA | | AATCCTGATA | |
| | AGG | | GGCGGCCGC | |
| CEL-122 | GGCCGCCAA | 105 | CGCGCCTTAA | 106 |
| | GTGTTGTTAT | | GAAAAAACAG | |
| | GTGAATGTTG | | ACCCAACATT | |
| | GGTCTGTTTT | | CACATAACAA | |
| | TTCTTAAGG | | CACTTGGCGG | |
| | | | CCGC | |
| CEL-71 | GGCCGCCGT | 107 | CGCGCCTTAA | 108 |
| | TCCGCGTGTT | | ATCGAAGTAA | |
| | ACTTCGATTT | | CACGCGGAA | |
| | AAGG | | CGGCGGCCG | |
| | | | C | |
| CEL-72 | GGCCGCCAT | 109 | CGCGCCTTAA | 110 |
| | GGCGAATCAT | | GAAAGATTAT | |
| | AATCTTTCTTA | | GATTCGCCAT | |
| | AGG | | GGCGGCCGC | |
| CEL-121 | CATGGTCCGC | 111 | CGCGCCTTAT | 112 |
| | GGCCGCCAC | | GCAGCCAGC | |
| | TCACAAGACC | | AGACGCTGAG | |
| | TCTACTCAGC | | TAGAGGTCTT | |
| | GTCTGCTGGC | | GTGAGTGGC | |
| | TGCATAAGG | | GGCCGCGGA | |
| | | | C | |
| PET-121 | GGCCGCCGG | 113 | CGCGCCTTAA | 114 |
| | TACGTCGGAT | | TTAAAAAAAG | |
| | CATATGATTA | | GCATAATCAT | |
| | TGCCTTTTTTT | | ATGATCCGAC | |
| | AATTAAGG | | GTACCGGCG | |
| | | | GCCGC | |

Preparation of GST-Peptide Fusion Proteins:

To express the GST-peptide fusion proteins, these new pGSTf constructs were transformed into BL21 A1 E. coli cells (Invitrogen). For each of the pGSTf constructs, one transformed colony was grown in 2.5 mL of LB broth containing 50 μg/mL of ampicillin (LB-Amp50) overnight at 37° C. and then re-grown in 50 mL of fresh LB-Amp50. When the cell density reached an OD₆₀₀ value of 0.8 to 1.0, L-arabinose was added to the culture to a concentration of 0.2% and protein expression was induced for 4 h at 37° C. Approximately 100 μL of the cells was spun down, and then lysed in 30 μL of SDS-PAGE loading buffer by boiling for 10 min. Protein expression was examined by running 10 μL of the cell lysate on a NuPAGE® SDS-PAGE gel (Invitrogen) to verify that the plasmid indeed produced the fusion protein. The B-PER GST fusion protein purification kit (Pierce, Rockford, Ill.) was used to purify the expressed proteins, following a protocol provided by the manufacturer. Briefly, E. coli collected from the 50-mL culture was lysed in 2.5 mL of B-PER Reagent with1 mM PMSF (phenylmethylsulphonyl fluoride) and 5 mM EDTA for 30 min at room temperature. Cell debris was removed by centrifugation. The soluble fraction (supernatant) was mixed with 1 mL of the 50% Immobilized Glutathione Slurry for 30 min and then centrifuged to collect the Glutathione gel bound with the fusion protein. The gel was resuspended in 0.25 mL of Wash Buffer, loaded on a Microfilter Spin Column, and centrifuged to remove the Wash Buffer. The slurry was then washed twice, using 0.5 mL portions of Wash Buffer. The fusion protein was eluted from the gel twice using 0.45 mL of Elution Solution. The two elution samples were combined and dialyzed in IEB buffer (50 mM Tris-HCl, pH 7.5, 50 mM NaCl, 5% glycerol). Protein quality was inspected by SDS-PAGE and the protein concentration was determined using the Bio-Rad protein assay reagent.

Measurement of MB₅₀ and Bₘₐₓ:

To obtain MB₅₀ and Bₘₐₓ data, the GST-peptide fusion proteins were titrated in the ELISA-based binding assay, described in Example 4, by adding 0.025, 0.05, 0.1, 0.15, 0.25, 0.4, and 0.5 nmol of the protein. For one assay reaction, the appropriate amount of substrate was placed in a well and blocked by filling the well with blocking buffer (0.1 M NaHCO₃, pH 8.6, 5 mg/mL BSA). The substrate and the well were washed 3 times with TBST (TBS-0.1% Tween-20), reacted with 200 μL of TBST solution containing the GST-peptide fusion protein to be tested and 1 mg/mL BSA for 15 min at room temperature, and finally washed 5 times with TBST. To detect the GST-peptide fusion protein that bound with its substrate, anti GST-HRP conjugate (Sigma Chemical Company) was diluted 2,500-fold with TBST, added to the well, and incubated for 1 h at room temperature. After 5 washings with TBST, 200 μL of ABTS (2,2′-azino-bis(3-ethylbenzthiazoline-6-sulfonic acid) substrate solution (0.4 mM), 50 mM sodium citrate, pH 4.0, 0.05% H₂O₂) was added to the well and the color reaction was developed for 25 min at room temperature. The OD₄₀₅ values were measured using a Victor-3 1420 Multiple Label Counter (Perkin Elmer, Shelton, Conn.).

In the assay, 37.5 μg of Carbon Black FW-18 (Degussa), 150 μg of Cromophtal® Yellow, 150 μg of Sunfast® Magenta, or 150 of μg Sunfast® Blue was used as the pigment binding substrate. A string of 20-mm fiber pulled from 100% cotton 419W fabric and 65/35 polyester/cotton 7436M fabric were used as fabric binding substrates in one reaction. A piece of 16 mm² Happermill Tidal MP paper was used as paper binding substrate. Sigmacell (Cellulose Type 20, Sigma Chemical Company), 600 μg, was used as the cellulose binding substrate. A string of 20-mm fiber pulled from 100% polyester fabric was used as polyester binding substrate. Each reaction was run in triplicate and the average of the OD₄₀₅ values were taken as a measure of the binding affinity of the peptide for the substrate. Reactions without any protein present were used as the blank reactions.

For each GST-peptide fusion protein, the average OD₄₀₅ values were plotted against the protein concentrations using GraphPad Prism 4.0 (GraphPad Software, Inc. San Diego, Calif.). The MB₅₀ and Bₘₐₓ values for the proteins were determined using Scatchard plots and are given in Table 11. In the table, a smaller MB₅₀ value indicates a stronger binding affinity, while a higher Bmax value shows that a larger amount peptide attaches to the substrate. An excellent peptide candidate should have a small MB₅₀ and high Bₘₐₓ, such as CB-122. Since GST itself did not show binding activity to any of the substrates tested (data not shown), this data demonstrates that the selected peptide candidates indeed possess substrate-specific binding activities and the peptides are still active after being integrated into a larger protein complex. However, this assay was carried out under non-equilibrium conditions because of the extensive washing used. Therefore, it may not be appropriate to compare this set of data to those obtained by other methods under different conditions.

TABLE 11
Summary of MB₅₀ and Bₘₐₓ Values for GST-Peptide
Fusion Proteins

| | Peptide in | | |
|---|---|---|---|
| | Fusion | | |
| Substrate | Protein | MB₅₀ | Bₘₐₓ (OD₄₀₅) |
| Carbon Black | CB-122 | 0.0149 | 0.9279 |
| Carbon Black | CB-121 | 0.1458 | 0.5302 |
| Carbon Black | CB-72 | 0.1632 | 0.4751 |
| Carbon Black | CB-71 | 0.2667 | 0.4465 |
| Sunfast ® Magenta | SM-71 | 0.1482 | 0.2264 |
| Sunfast ® Magenta | SM-121 | 0.1753 | 0.4403 |
| Sunfast ® Magenta | SM-72 | 0.3043 | 0.4513 |
| Cromophtal ® Yellow | CY-72 | 0.0948 | 0.2526 |
| Cromophtal ® Yellow | CY-71 | 0.1195 | 0.2466 |
| Cromophtal ® Yellow | CY-121 | 0.1881 | 0.2957 |
| Cromophtal ® Yellow | CY-125 | 0.4770 | 0.4583 |
| Sunfast ® Blue | SB-72 | 0.1618 | 0.4884 |
| Sunfast ® Blue | SB-71 | 0.1678 | 0.3048 |
| Sunfast ® Blue | SB-121 | 0.1704 | 0.2766 |
| Sunfast ® Blue | SB-128 | 1.0750 | 0.2925 |
| Cotton | COT-71 | 0.2895 | 0.4084 |
| Cotton | COT-72 | 0.3021 | 0.3117 |
| Cotton/PET | P/C-71 | 0.2630 | 0.2860 |
| Cotton/PET | P/C-72 | 0.3092 | 0.3415 |
| Paper | HCP-73 | 0.0214 | 0.1349 |
| Paper | HCP-74 | 0.0218 | 0.1758 |
| Cellulose | CEL-122 | 0.1252 | 0.2985 |
| Cellulose | CEL-71 | 0.1369 | 0.3160 |
| Cellulose | CEL-72 | 0.1383 | 0.3256 |
| Cellulose | CEL-121 | 0.3064 | 0.1064 |
| Polyester | PET-121 | 0.2742 | 0.3303 |

### Example 7

### Measurement of Peptide-Substrate Binding Energy Using Flow Microcalorimetry

The purpose of this Example was to measure the strength of the interaction, specifically, the molar heat of adsorption, of the pigment-binding and print medium-binding peptides with their respective substrate using flow microcalorimetry.

Heats of adsorption and desorption for the interaction of the binding peptides with their respective substrate were measured with a Model 4034 flow microcalorimeter (Microscal, London, Ltd.). The flow microcalorimeter consists of a constant temperature metal block which has an inlet and outlet connector which come together inside the unit to form a sample cavity or cell with a volume of 0.17 cm³. A set of two thermistors is embedded in the metal block along with a second set of two thermistors in the sample cavity. By means of a Wheatstone bridge circuit, heat changes in the microcalorie range can be measured. The outlet tube is fitted with a 25 micron filter, on which the sample bed sits, and also includes a calibration coil. The analog data stream from the Wheatstone circuit was sampled at one-second intervals, digitized and sent to an attached computer for storage and subsequent analysis. All experiments were run at 24.5±0.5° C.

A Waters Model 2410 differential refractometer (Waters Corp., Milford, Mass.) was located downstream of the flow microcalorimeter to monitor mass transfer into and out of the carrier solution. Since no detectable mass transfer was observed for the second adsorption/desorption cycle, this second cycle was used as the blank for the downstream detector. Integrating the time indexed difference between the blank and sample run through the downstream detector produced a concentration peak. Calibration of the peak area was accomplished by injecting the peptide-containing solution into a sample loop of known volume on the downstream detector.

The substrate to be tested, specifically, cellulose fiber (Sigma Chemical Co.) or polyethylene terephthalate (polyester) was deposited in fiber or particulate form in the sample cell of the instrument. Water was used as the carrier solvent. The carrier solvent was pumped through the sample cell until equilibrium was attained, as evidenced by no change in heat flow into or out of the sample cell. After equilibrium was attained, the solvent stream was switched to one that contained a known concentration of the specific binding peptide. The adsorption of the peptide onto the substrate resulted in an exothermic peak and a decrease of the peptide concentration in the carrier solvent, as determined downstream using the differential refractive index monitor. The flow of the peptide-containing solvent was continued until equilibrium was attained. Then the solvent stream was switched back to the pure solvent and the desorption of the peptide from the substrate, if any, was monitored. For comparison, a traditional acrylic binder (polymethacrylic acid) was tested for its binding to the substrate using the same method.

The surface area of the print media substrates used in these studies was measured in order to calculate the mass transfer of the binding peptides per unit surface area of the substrate surface using the data collected by the refractive index monitor. The surface area determinations were done using dinitrogen adsorption measurements at 77.3° K. using a Micromeritics ASAP® Model 2400/2405 porosimeter (Micromeritics Inc., Norcross, Ga.). Samples were degassed overnight at 60° C. prior to data collection. The surface area measurements were made using a five-point adsorption isotherm collected over the relative pressures (P/P₀) of 0.05 to 0.20 and were analyzed via the BET method, as described by Brunauer et al. (J. Am. Chem. Soc. 60:309 (1938)). The mass transfer value in μmol/m² was calculated by dividing the amount of the sample adsorbed onto the substrate, as determined from the measurements made with the refractive index monitor, by the surface area of the substrate. The resulting mass transfer values are given in Table 12. No significant change in concentration for any of the samples was observed during the desorption step. This result is consistent with irreversible adsorption.

The heats of adsorption from the microcalorimetry determinations are given in Table 13 in units of millijoules per square meter (mJ/m²). These values were converted to the molar heats of adsorption, given in units of kilocalories per mole (kcal/mol) in Table 13, by dividing the heat of adsorption by the mass transfer value and using the appropriate conversion factors to obtain the desired units. The negative values for the heats of adsorption and the molar heats of adsorption in the table indicate that the process was exothermic, i.e., heat was given off as a result of the binding. As can be seen from the data in the table, all the molar heats of adsorption for the peptides are equal to or greater than 20 kcal/mol and are comparable to the value obtained for the methacrylate control. No significant heat of desorption was observed with any of the samples, indicating that the adsorption in all cases was essentially irreversible.

The results of this study indicate that the peptides of the present invention compare favorably with the traditional acrylic binder control in terms of the strength of the binding interaction.

TABLE 12
Mass Transfer Results for Peptide-Substrate Interaction

| | | | Mass |
|---|---|---|---|
| Sample/ | | | Transfer |
| Concentration | | Surface Area | Value |
| (mM) | Substrate | (m²/g) | (μmol/m²) |
| CEL-71 (12.1) | Cellulose | 1.0 | 6.17 |
| Run 1 | Fiber | | |
| CEL-71 (12.1) | Cellulose | 1.0 | 6.92 |
| Run 2 | Fiber | | |
| CEL-121 (11.8) | Cellulose | 1.0 | 2.70 |
| | Fiber | | |
| COT-71 (11.2) | Cellulose | 1.0 | 5.43 |
| | Fiber | | |
| PET-121 (8.13) | PET | 0.071 | 28.7 |
| Polymethacrylic | Cellulose | 1.0 | 8.56 |
| Acid, 10 mer | Fiber | | |
| (11.4) | | | |

TABLE 13
Heats of Adsorption for Peptide-Substrate Interaction

| Sample/ | | Heat of | Molar Heat of |
|---|---|---|---|
| Concentration | | Adsorption | Adsorption |
| (mM) | Substrate | (mJ/m²) | (kcal/mol) |
| CEL-71 (12.1) | Cellulose | −628 | −24.3 |
| Run 1 | Fiber | | |
| CEL-71 (12.1) | Cellulose | −835 | −28.8 |
| Run 2 | Fiber | | |
| CEL-121 (11.8) | Cellulose | −503 | −44.5 |
| | Fiber | | |
| COT-71 (11.7) | Cellulose | −454 | −20.5 |
| | Fiber | | |
| PET-121 (8.13) | PET | −6080 | −50.6 |
| Polymethacrylic | Cellulose | −991 | −27.7 |
| Acid, 10 mer | Fiber | | |
| (11.4) | | | |

### Example 8

### Preparation of a Pigment-Binding Peptide/Linker Diblock Dispersant

The purpose of this Example was to prepare a diblock dispersant consisting of a pigment-binding peptide and a polyproline linker. The polyproline linker was synthesized by the polymerization of the N-carboxyanhydride of L-proline in situ via activation through the N-terminal amine of the carbon black-binding peptide CB-71, given as SEQ ID NO:7.

Synthesis of L-Proline N-Carboxyanhydride:

The N-carboxyanhydride of L-proline was synthesized from L-proline by phosgenation in dry tetrahydrofuran (THF). A 3,000 mL round bottom, four-necked flask with a thermocouple well was fitted with a calibrated addition funnel that was topped with a small dry ice cold finger. A second, larger cold finger was also fitted to the flask. A magnetic stirring bar was placed in the flask, which was charged with 1500 mL of dry THF 20 and 23.02 g (200 mmol) of L-proline (Sigma Chemical Co., Saint Louis, Mo.). Liquid phosgene (28 mL) was added in two 14 mL portions to the reaction and the reaction mixture was stirred until a clear solution was obtained. The solvent was then vacuum distilled at 40° C. or less to a volume of about 40 mL. The clear oil obtained was then transferred to a dry box and 100 mL of hexanes (EM Science, Gibbstown, N.J.) was added. After swirling to wash the oil, the hexanes were decanted and 100 mL of THF at −20° C. was added. An additional 100 mL of cold THF containing triethylamine (20.2 g) was then added, causing the precipitation of triethylamine hydrochloride salts that were removed by filtration. Hexanes (150 mL) were added to the filtrate and the solution was placed in a refrigerator at −20° C. for 24 h. The resulting white crystals (2 crops) were collected by vacuum filtration. The resulting yields were 13.3 g and 9.1 g, respectively.

Synthesis of Carbon Black-Binding Peptide/Polyproline Diblock Dispersant:

One gram of the carbon black-binding peptide CB-71 (SEQ ID NO:7) (obtained from SynPep Corporation, Dublin, Calif.) was dissolved in 10 mL of dimethylformamide (DMF) in a 100 mL round bottom flask fitted with a septum and magnetic stir bar. Proline N-carboxyanhydride (1.2 mL of a 1.0 mmol/mL solution in DMF), prepared as described above, was added all at once with rapid stirring at room temperature. The reaction mixture was stirred at room temperature for 24 h, after which time 8 mL of DMF was added, followed by the drop-wise addition of an additional 10.8 mL of the proline N-carboxyanhydride solution over a 4 h period using a Harvard Model 44 syringe pump (Harvard Apparatus Inc., Holliston, Mass.). The reaction was stirred overnight and then the DMF was removed using a freeze-drier. The product was washed with hexanes, collected by filtration and vacuum-dried at 40° C. The yield was 1.69 g of a beige-colored powder. The product was analyzed using gel permeation chromatography (GPC) and the number-average molecular weight (Mₙ) was found to be 2170 g/mol. The theoretical molecular weight for the product is 1800 g/mol.

### Example 9

### Preparation and Performance Testing of Pigment-Binding Peptide/Linker Diblock Dispersants

The purpose of this Example was to prepare diblock dispersants consisting of a pigment-binding peptide and a polyproline linker and to test their performance as pigment dispersants. The polyproline linker was synthesized by the polymerization of the N-carboxyanhydride of L-proline in situ via activation through the N-terminal amine of the carbon black-binding peptide CB-121, given as SEQ ID NO:8 or the magenta-binding peptide SM-71, given as SEQ ID NO:18.

A. Synthesis of Carbon Black-Binding Peptide/Polyproline Diblock Dispersant:

One gram of the carbon black-binding peptide CB-121 (SEQ ID NO:8) (obtained from SynPep Corporation, Dublin, Calif.) was dissolved in 10 mL of DMF in a 100 mL round bottom flask fitted with a septum and magnetic stir bar. Proline N-carboxyanhydride, prepared as described in Example 8, (0.8 mL of a 1 mmol/mL solution in DMF) was added all at once with rapid stirring at room temperature. The reaction mixture was stirred at room temperature for 24 h, after which time 12 mL of DMF was added, followed by the drop-wise addition of an additional 7.2 mL of the proline N-carboxyanhydride solution over a 4 h period using a Harvard Model 44 syringe pump. The reaction was stirred overnight and then DMF was removed at reduced pressure. The product was washed with hexanes, collected by filtration and vacuum-dried at 40° C. The yield was 1.36 g of a beige-colored powder. The product was analyzed by GPC and the number-average molecular weight (Mₙ) was found to be 2850 g/mol. The theoretical molecular weight of the product is 2260 g/mol.

Dispersion Testing of the Carbon Black-Binding Peptide/Polyproline Diblock Dispersant:

Qualitative testing of the ability of the dispersants of the present invention was carried out using a shake bottle test. The test consisted of combining the dispersant of interest with a pigment and other additives, including an inert abrasive particle, and vigorously shaking the mixture, as described below. The effectiveness of the dispersant was determined by examining the viscosity and deflocculation of the mixture after shaking.

One gram of the carbon black binding-peptide CB-121/polyproline diblock dispersant, prepared as described above, was suspended in 18.73 g of deionized water containing 0.27 g of a 10% aqueous potassium hydroxide solution. The resulting solution was slightly cloudy. A 2 oz screw-capped vial was charged with 8 g of the dispersant solution, 7.5 g of zirconia beads, 0.1 g defoamer (Nopco NDW, Akron Ohio), 1.0 g of FW-18 carbon black pigment and 3.5 g of deionized water. The vial was tightly capped, sealed with tape and placed in a paint can filled with cushioning material. This assembly was then agitated on a paint mixer (Red Devil, Union N.J.) for 30 min. After that time, the dispersion was qualitatively examined for viscosity and the degree of flocculation as determined by the characteristics of the thin film that was formed when the dispersion flowed down the wall of the vial. This diblock dispersant was judged to have good dispersion properties for the carbon black pigment. A dispersion was judged to be good if the viscosity of the liquid was low and if a uniform thin film formed while draining down the sides of the glass vial without large patches of color or channels forming. It must be emphasized that the ability of any given polymer to behave as an effective dispersant is strongly dependent upon the overall dispersion formulation including additives, pH and the degree of neutralization of acid or base groups on the polymer.

This carbon black binding-peptide CB-121/polyproline diblock dispersant was also tested using carbon black pigments other than the FW-18 pigment used to identify the pigment-binding peptide, specifically, Degussa 90, Degussa 150T and two lots of NIPex® 180 (all from Degussa, Piscataway, N.J.). The CB-121/polyproline diblock dispersant was not effective in dispersing these other pigments. However, other carbon black binding peptide dispersants, specifically, CB-71 (SEQ ID NO:7)/polyproline₁₀, and CB-72 (SEQ ID NO:6)/polyproline₁₀, prepared in the same manner as described above, functioned as effective dispersants for most of these other carbon black pigments. In general, it is best to select a pigment-binding peptide for the specific pigment to be used in the formulation.

For comparison, this carbon black-binding peptide/polyproline dispersant was tested as a dispersant for the pigment Sunfast® Magenta 122 at the same conditions. This diblock peptide did not disperse the Sunfast Magenta pigment, but instead yielded a highly viscous mixture. This result demonstrates the selectivity of the carbon black-binding peptide/polyproline dispersant

B. Synthesis of a Magenta-Binding Peptide/Polyproline Diblock Dispersant:

One gram of the magenta binding peptide SM-71 (SEQ ID NO:18) (obtained from SynPep Corporation, Dublin, Calif.) was dissolved in 10 mL of DMF in a 100 mL round bottom flask fitted with a septum and magnetic stir bar. Proline N-carboxyanhydride, prepared as described in Example 8, (0.8 mL of a 1 mmol/mL solution in DMF) was added all at once with rapid stirring at room temperature. The reaction mixture was stirred at room temperature for 24 h, after which time 8 mL of DMF was added, followed by the drop-wise addition of an additional 11.2 mL of the proline N-carboxyanhydride solution over a 4 h period using a Harvard Model 44 syringe pump. The reaction was stirred overnight and then DMF was removed at reduced pressure. The product was washed with hexanes, collected by filtration and vacuum-dried at 40° C. The yield was 1.79 g of a beige-colored powder. The product was analyzed by GPC and the number-average molecular weight (Mₙ) was found to be 2460 g/mol. The theoretical molecular weight of the product is 1745 g/mol.

Dispersion Testing of the Magenta-Binding Peptide/Polyproline Diblock Dispersant

The magenta-binding peptide/polyproline diblock dispersant (0.25 g), prepared as described above, was suspended in 10 mL of deionized water containing 0.5 mL of 10% aqueous KOH solution in a 2 oz screw-capped vial. Sunfast® Magenta 122 pigment (0.625 g), sand (7.5 g), and defoamer (0.1 g) were added and the vial was capped and shaken for 30 min as described above. A very good dispersion was obtained, exhibiting low viscosity and no flocculation. For comparison, this magenta-binding peptide/polyproline peptide dispersant was tested as a dispersant for the pigment carbon black FW18 at the same conditions. This diblock peptide did not disperse the carbon black pigment, demonstrating its selectivity.

### example 10

### Preparation of a Print Medium-Binding Peptide/Hydrophobic Linker Diblock Dispersant

The purpose of this Example was to prepare a diblock dispersant consisting of a cotton-binding peptide and a hydrophobic poly gamma benzyl glutamate linker. This Example illustrates the capability of selected print media-binding peptides to serve both as a hydrophilic linker for aqueous dispersions and as a specific print medium-binder. The poly gamma benzyl glutamate linker was synthesized by the polymerization of the N-carboxyanhydride of gamma benzyl glutamate in situ via activation through the N-terminal amine of the cotton-binding peptide COT-71, given as SEQ ID NO:30.

Synthesis of Gamma Benzyl Glutamate N-Carboxyanhydride:

Gamma benzyl glutamate N-carboxyanhydride was prepared by phosgenation, as described in Example 8 for the preparation of L-proline N-carboxyanhydride, using gamma-benzyl-L-glutamate. A 1,000 mL round bottomed, four-necked flask with a thermocouple well was fitted with a calibrated addition funnel that was topped with a small dry ice cold finger. A second, larger cold finger was also fitted to the flask. A magnetic stirring bar was placed in the flask, which was charged with 500 mL of dry THF and 50 g (200 mmol) of gamma-benzyl-L-glutamate (Fluka Chemical Corp., Milwaukee, Wis.). Liquid phosgene (24 mL) was added in two 12 mL portions to the reaction while heating at reflux. The reaction mixture was stirred at reflux until a clear solution was obtained. The solvent was then vacuum distilled at 40° C. or less to dryness. The white crystalline product was then transferred to a dry box and 800 mL of hexanes was added. After swirling in hexanes, the crystals were collected by filtration. The product was recrystallized from THF/hexane mixtures. The resulting white crystals (2 crops) were collected by vacuum filtration giving 53.2 g (95.9% yield).

Synthesis of Cotton-Binding Peptide/Poly Gamma Benzyl Glutamate Diblock Dispersant:

In a dry box, 1.0 g of the cotton-binding peptide COT-71 (SEQ ID NO:30) (obtained from SynPep Corporation, Dublin, Calif.) was dissolved in 10 mL of distilled and dried DMF in a 100 mL round bottom flask fitted with a septum and a magnetic stir bar. Then, 1.2 mL of a 1 mmol/mL solution of gamma-benzyl L-glutamate N-carboxyanhydride in DMF, prepared as described above, was added all at once with rapid stirring at room temperature. This reaction mixture was stirred at room temperature over 72 h. After this time, 8 mL of DMF was added and an additional 10.8 mL of the gamma-benzyl L-glutamate N-carboxyanhydride solution was added slowly over a period of 4 h using a Harvard Model 44 syringe pump. This reaction mixture was stirred for an additional 48 h. After this time, the solvent was evaporated to dryness under vacuum. The recovered, dried product was washed with hexanes, allowed to air dry, and then dried in vacuum at 40° C. The yield was 2.99 g of an off-white powder. The product was analyzed using GPC and the number-average molecular weight (Mₙ) was found to be 4230 g/mol. The theoretical molecular weight of the product is 3482 g/mol.

### Example 11

### Preparation of a Carbon Black-Binding Peptide/Print Medium-Binding Peptide Diblock Dispersant

The purpose of this Example was to prepare a diblock dispersant consisting of cotton-binding peptide COT-71 (SEQ ID NO:30) and carbon black-binding peptide CB-71 (SEQ ID NO:7).

4,4′ Methylenebis(phenylisocyanate) (0.308 g) was dissolved in 10 mL of DMF in a 100 mL round bottom flask, fitted with a septum and a magnetic stir bar. A solution of 1.0 g of carbon black binding-peptide CB-71 (SEQ ID NO:7) in 10 mL of DMF was added drop-wise over a 2 h period at room temperature using a syringe pump and the reaction mixture was stirred for an additional 4 h at room temperature after the addition was complete. Cotton-binding peptide COT-71 (SEQ ID NO:30) (1.070 g) in 10 mL of DMF was added all at once with rapid stirring and the resulting solution was stirred at room temperature for 24 h. Then, the solvent was removed by vacuum distillation, the product was washed with hexanes and collected by filtration. The sample was vacuum-dried at 40° C. to give 2.02 g of an off-white powder. The product was analyzed using GPC and the number-average molecular weight (Mₙ) was found to be 2430 g/mol. The theoretical molecular weight of the product is 1960 g/mol.

### Example 12

### Washfastness Testing of a Cotton-Binding Peptide

The purpose of this Example was to demonstrate the washfastness durability of the cotton-binding peptide COT-71, given as SEQ ID NO:30, on cotton and to compare it to a polymethacrylate control. Both the cotton-binding peptide and the polymethacrylate were coupled to a dye to enable a spectrophotometric determination.

### Preparation of 4-(2-(4-isocyanatophenyl)diazenyl)benzenamine. (Isocyanate Functionalized Disperse Orange 3)

Phosgene (25.4 mL) was dissolved in toluene to make a 35% by weight solution. Disperse Orange 3 (4-(2-(4-nitrophenyl)diazenyl)benzenamine, obtained from Aldrich (Cat. No. 36,479-7), (14.25 g) was suspended in toluene and the previously prepared phosgene solution was added drop-wise over five minutes. The reactants were warmed to 100° C. for 2 h and then, allowed to cool to room temperature. The resulting solid was filtered and washed with toluene to give 11.33 g (84.5% yield) of the desired isocyanate derivative.

Preparation of Dye-Coupled Cotton-Binding Peptide:

4-(2-(4-isocyanatophenyl)diazenyl)benzenamine, obtained from Aldrich (Cat. No. 25,643-9), (0.70 g) was dissolved in DMF (50 mL). The cotton binding peptide COT-71 (1.45 g), given as SEQ ID NO:30, was dissolved in 25 mL of DMF and added to the above solution with vigorous stirring at room temperature. The reaction was stirred for four days. The reaction solution was filtered to remove undissolved solids and the filtrate was diluted with diethylether to precipitate a dark reddish brown powder, which was a vacuum dried (0.5 g, 23% yield).

Preparation of Dye-Coupled Methacrylic Acid Oligomer:

Polymethacrylic acid oligomer (DP=10; 2.0 g) was prepared via group transfer polymerization of trimethylsilyl methacrylate, followed by removal of the trimethylsilyl groups using methods similar to those described by Ma et al. in U.S. Pat. No. 5,085,698. The oligomer was dissolved in oxaloyl chloride (4 mL) and the mixture was refluxed for 30 min. Excess oxaloyl chloride was removed by vacuum distillation. Disperse Orange 3, (4-(2-(4-nitrophenyl)diazenyl)benzenamine), obtained from Aldrich (Cat. No. 36,479-7), (1.15 g) was dissolved in 20 mL of THF and this solution was added to the dried acid chloride above. After the acid chloride redissolved, the solution was heated at reflux for 30 min. Then, 5 mL of water was added and the mixture was heated for 30 min at reflux. The solution was filtered and the filtrate was concentrated in vacuum to yield 2.01 g of crude product. This solid was divided into small portions and purified by column chromatography on silica gel using 9:1 v/v chloroform/methanol as the eluting solvent.

Washfastness Durability Testing:

Solutions of the dye-coupled cotton-binding peptide and the dye-coupled methacrylic acid oligomer were prepared at a concentration of 20% solids in dimethylformamide. Colored spots of about 2.5 to 5 cm in diameter were prepared by spraying these solutions onto 100% cotton fabric swatches (10 cm×10 cm). The fabric samples were then conditioned by standing at room temperature for 24 h or by heating at 70° C. for 10 min prior to fabric testing.

Washfastness durability was determined using an accelerated laundering test (American Association of Textile Chemists and Colorists (AATCC) test method 61-1996). Color intensity measurements before and after accelerated laundering were determined spectrophotometrically by placing the colored spot region of the fabric into the photosensor and calculating L*, a* and b* parameters representing the photometer response. An initial baseline L* value was measured for the unspotted fabric and all measurements were the average of three individual determinations. Delta E values were calculated from the equation 1 below:
Delta E=((L*₁−L*₂)²+(a₁−a₂)²+(b₁−b₂)²)^{1/2} (1)
where L*=the lightness variable and a* and b* are the chromaticity coordinates of CIELAB colorspace as defined by the International Commission of Illumination (CIE) (Minolta, Precise Color Communication—Color Control From Feeling to Instrumentation, Minolta Camera Co., 1996). The Delta E value correlates with color loss; therefore, smaller values are indicative of better washfastness durability.

The results are presented in Table 14. As can be seen from the data in the table, the washfastness of the cotton-binding peptide was better than that of the methacrylate control.

TABLE 14
Results of Washfastness Testing

| Sample | Fabric | Cure Conditions | Delta E |
|---|---|---|---|
| COT-71 | 100% cotton | room | 17.92 |
| | | temperature | |
| COT-71 | 100% cotton | 10 min 70° C. | 18.40 |
| Methacrylate | 100% cotton | room | 38.44 |
| Control | | temperature | |
| Methacrylate | 100% cotton | 10 min 70° C. | 28.03 |
| Control | | | |

### Example 13

### Preparation of a Cotton-Binding Peptide/Hydrophobic Linker Diblock Dispersant

The purpose of this Example was to prepare a diblock dispersant consisting of a cotton-binding peptide and an poly(benzylmethacrylate) linker, which serves as a pigment binding domain. A Poly(benzylmethacrylate) oligomer (DP=9), terminated with a single hydroxyethylmethacrylate unit, was capped via the terminal hydroxyl group with bis(4-isocyanatophenyl)methane according to the procedure below. This provided a reactive intermediate that was coupled directly to the cotton binding peptide.

Preparation of Isocyanate Capped Poly(Benzylmethacrylate):

Poly (benzyl methacrylate/hydroxyethyl methacrylate) was prepared via group transfer polymerization using the general method described by Ma et al., supra, and 10 g was dissolved in 50 mL of THF. Bis(4-isocyanatophenyl)methane, obtained from Aldrich (Cat. No. 25,643-9), (12 g) was also dissolved in 50 mL of THF, and then was added drop-wise to the polyacrylate solution over several hours with vigorous stirring. After the addition was complete, 10 mL of dry, ethyl amine was added and the reactants were allowed to stir overnight at room temperature. The isocyanate capped product was then obtained by careful fractional precipitation by addition of hexanes. The precipitated polymer was collected, dissolved in diethyl ether and freshly precipitated once more by the addition of hexanes. After drying the purified product, the product weight was 5.89 g. The identity of the purified product was confirmed using NMR and MALDI mass spectrometry.

Synthesis of Cotton Binding Peptide (COT-71)/Benzylmethacrylate Diblock Dispersant:

A synthetic peptide, given as SEQ ID NO:115, containing the COT-71 cotton binding peptide sequence (SEQ ID NO:30) and an N-terminal lysine separated from the cotton binding domain by a tetraproline spacer was prepared by Synpep Corporation using the solid phase synthesis techniques described supra. In a drybox, 0.72 g of the vacuum dried peptide was dissolved in dry dimethylformamide. The isocyanate capped acrylate (1.04 g) described above was charged into a 40 mL screw capped bottle fitted with a rubber septum and containing a magnetic stirring bar. The peptide solution was added to the bottle all at once. The bottle was then placed into a heated block with stirring and held at 60° C. for 6 h, after which time it were removed and the reaction mixture was allowed to stir at room temperature overnight. The solution was then passed through a 1 μm filter, and then the solvent was removed by vacuum distillation. The product was washed several times with diethylether to yield 1.4 g of purified product.

### Example 14

### Washfastness Testing of Carbon Black Ink Having a Cotton-Binding Peptide/Hydrophobic Linker Diblock Dispersant

The purpose of this Example was to demonstrate the washfastness durability of a carbon black ink having a cotton-binding peptide/poly(benzylmethacrylate) dispersant and to compare it to a carbon black ink having a traditional acrylate diblock copolymer dispersant.

Carbon Black Ink Formulation:

Carbon black ink dispersions were prepared using the bottle shake method described in Example 9. Details of the ink formulations are described in Table 15. The peptide-based dispersant formulation was prepared using the cotton-binding peptide (COT-71)/benzylmethacrylate diblock dispersant described in Example 13. The acrylate-based dispersant formulation was prepared using an acrylate diblock copolymer consisting of benzylmethacrylate₁₃/methacrylic acid₁₀. The dispersions were made using Printex 150T Carbon Black Pigment (Degussa, Piscataway, N.Y.). Sand (0.5 g) was used as the abrasive medium throughout.

TABLE 15
Carbon Black Ink Dispersion Formulations

| | | | | | 10% | |
|---|---|---|---|---|---|---|
| | DMF | Dispersant | Pigment | Defoamer | KOH | Water |
| Formulation | (g) | (g) | (g) | (g) | (g) | (g) |
| Peptide- | 4 | 0.25 | 0.375 | 0.1 | 0.039 | 6 |
| based | | | | | | |
| Acrylate- | 4 | 0.25 | 0.375 | 0.1 | 0.435 | 6 |
| based | | | | | | |

Washfastness Durability Testing of Carbon Black Inks:

The carbon black inks were applied to 10 cm×10 cm square cotton fabrics using a metal roller and were allowed to dry overnight. They were then tested using AATCC test method 61-1996 after a cold water wash step without detergent or after heat-setting at 160° C. for 2 min between heated platens, followed by a cold water wash step without detergent. Delta E values were measured as described in Example 12 and are given in Table 16. The results indicate that the washfastness durability of the carbon black ink having the peptide-based dispersant is comparable to that of the ink having the acrylate-based dispersant.

TABLE 16
Results of Washfastness Testing of Carbon Black Inks

| Formulation | Fabric | Cure Conditions | Delta E |
|---|---|---|---|
| Peptide-based | 100% Cotton | 2 min 160° C. | 16.01 |
| Peptide-based | 100% Cotton | cold water wash | 32.98 |
| Acrylate-based | 100% Cotton | 2 min 160° C. | 21.40 |
| Acrylate-based | 100% Cotton | cold water wash | 17.06 |

### Example 15

### Preparation of a Carbon Black-Binding Peptide/Peptide Linker/Cellulose-Binding Peptide Triblock Dispersant

The purpose of this Example was to prepare a triblock protein dispersant consisting of a carbon black pigment-binding peptide, a hydrophilic peptide linker, and a cellulose-binding peptide using recombinant DNA and molecular cloning techniques.

The designed triblock dispersant has the structure of CB-72 (SEQ ID NO:6)/ST-Linker/CEL-121 (SEQ ID NO:37). The ST-Linker is a natural interdomain linker in cellobiohydrolase I of the plant pathogen fungus Aspergillus, having the sequence given by SEQ ID NO:45.

Design and Synthesis of TBP1 Gene:

To make the synthetic gene, designated as TBP1, to produce the CB-72 (SEQ ID NO:6)/ST-Linker/CEL-121 (SEQ ID NO:37) triblock protein dispersant, the oligonucleotides listed in Table 17 were synthesized by Sigma-Genosys (Woodlands, Tex.).

TABLE 17

Oligonucleotides Used to Prepare the TBP1 Gene

| Oligo | | SEQ |
|---|---|---|
| Name | Sequence (5′-3′) | ID NO: |
| TBP1(+)1 | GGATCCATCGAAGGTCGTTTCCACGAAAACTG | 46 |
| | GCGTCTGGTGGCGGTACCTCTACTTCCAAAGC | |
| | TTCCACCACTACGACTTCTAGCAAAACCACCA | |
| | CTACAT | |
| TBP1(+)2 | CCTCTAAGACTACCACGACTACCTCCAAAACC | 47 |
| | TCTACTACCTCTAGCTCCTCTACGGGCGGTGG | |
| | CACTCACAAGACCTCTACTCAGCGTCTGCTGG | |
| | CTGCATAA | |
| TBP1(−)1 | TTATGCAGCCAGCAGACGCTGAGTAGAGGTCT | 48 |
| | TGTGAGTGCCACCGCCCGTAGAGGAGCTAGAG | |
| | GTAGT | |
| TBP1(−)2 | AGAGGTTTTGGAGGTAGTCGTGGTAGTCTTAG | 49 |
| | AGGATGTAGTGGTGGTTTTGCTAGAAGTCGTA | |
| | GTGGT | |
| TBP1(−)3 | GGAAGCTTTGGAAGTAGAGGTACCGCCACCAG | 50 |
| | ACGGCCAGTTTTCGTGGAAACGACCTTCGATG | |
| | GATCC | |

Each oligonucleotide was phosphorylated with ATP using T4 polynucleotide kinase. The resulting oligonucleotides were mixed, boiled for 5 min, and then cooled to room temperature slowly. Finally, the annealed oligonucleotides were ligated with t4 DNA ligase to give synthetic DNA fragment TBP1, given as SEQ ID NO:51, which encodes the CB-72/ST-Linker/CEL-121 triblock protein dispersant, given as SEQ ID NO:52.

Construction of plNK1 Expression Plasmid:

TBP1 was integrated into the Gateway™ Technology system for protein over-expression (Invitrogen, Carlsbad, Calif.). In the first step, 2 μL of the TBP1 ligation mixture was used in a 50 μL PCR reaction. Reactions were catalyzed by pfu DNA polymerase (Stratagene, La Jolla, Calif.), following the standard PCR protocol. Primer 5′TBP1 (5′-CAC CGG ATC CAT CGA AGG TCG T-3′), given as SEQ ID NO:53, and primer 3′TBP1 (5′-TCA TTA TGC AGC CAG CAG CGC-3′), given as SEQ ID NO:54, were used for amplification of the TBP1 fragment. Due to the design of these primers, an additional sequence of CACC and another stop codon TGA were added to the 5′ and 3′ ends of the amplified fragments. The amplified TBP1 was directly cloned into pENTR/D-TOPO vector using Invitrogen's pENTR directional TOPO® cloning kit, resulting in Gateway entry plasmid pENTR-TBP1. This entry plasmid was propagated in One Shot TOPO10 E. coli strain (Invitrogen). The accuracy of the PCR amplification and cloning were determined by DNA sequencing at the DuPont Sequencing Facility. Finally, the entry plasmid was mixed with pDEST17 (Invitrogen). LR recombination reactions were catalyzed by LR Clonase™ (Invitrogen). The destination plasmid, plNK1 was constructed and propagated in the DH5α E. coli strain. The accuracy of the recombination reaction was determined by DNA sequencing. All reagents for LR recombination reactions were provided in Invitrogen's E. coli expression system with Gateway™ Technology kit. The entire process described above followed Invitrogen's instructions.

The destination plasmid plNK1 contains the coding regions for recombinant protein 6H-TBP1, given as SEQ ID NO:55, which is an 11.6 kDa protein. This protein sequence includes a 6×His tag and a Factor Xa recognition site. Following this N-terminal sequence, 6H-TBP1 contains a tri-block protein TBP1, which has the structure of CB-72/ST-Linker/CEL-121, given as SEQ ID NO:52. TBP1 can be released from 6H-INK1 by Factor Xa treatment.

Production of 6H-TBP1 Protein:

The expression plasmid plNK1 was transformed into the BL21-Al E. coli strain (Invitrogen). To produce the recombinant protein, 50 mL of LB-carbenicillin broth (10 g/L bacto-tryptone, 5 g/L bacto-yeast extract, 10 g/L NaCl, 50 mg/L carbenicillin, pH 7.0) was inoculated with one colony of the transformed bacteria and the culture was shaken at 37° C. until the OD₆₀₀ reached 0.6. The expression was induced by adding 0.5 mL of 20% L-arabinose to the culture and shaking was continued for another 4 h. Approximately 0.15 g of bacteria was collected by centrifuging the culture at 10,000×g for 15 min.

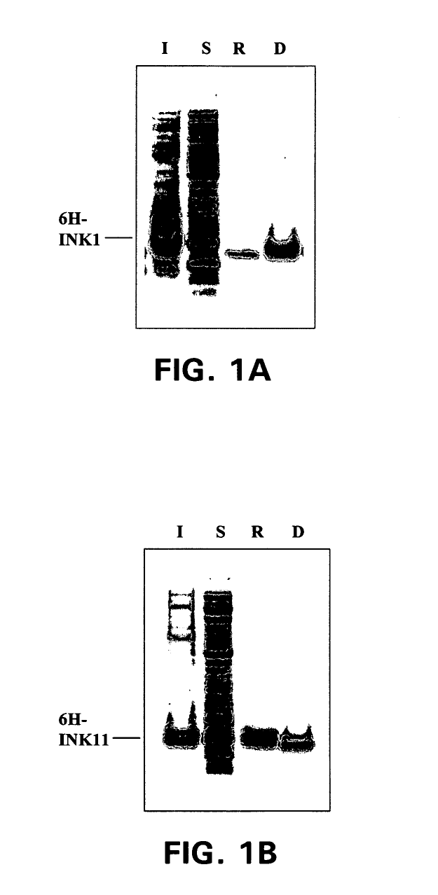

The collected bacteria was suspended in 4 mL Cellytic B-bacterial cell lysis/extraction reagent (Sigma Chemical Company, St. Louis, Mo.) containing 0.2 mg/mL lysozyme (Sigma Chemical Company). The bacterial cells were lysed during a 30-min incubation at room temperature. Soluble and insoluble fractions were separated by a 15-min centrifugation at 10,000×g at 4° C. The insoluble fraction was dissolved in 5 mL lysis buffer (8 M urea, 0.1 M NaH₂PO₄, 0.01 M Tris-HCl, pH 8.0). Aliquots (13 μL) of the soluble fraction and the dissolved insoluble fraction were subjected to 10% SDS-PAGE and the resulting gel was stained with Coomassie brilliant blue G-250. The electrophoresis results are shown in FIG. 1A, where lanes I and S indicate the insoluble fraction and the soluble fraction, respectively. These results indicate that the protein 6H-TBP1 was expressed in the insoluble fraction (see lanes S and I in FIG. 1A).

To purify the recombinant protein, 1 mL of Ni-NTA Superflow resin (QIAGEN) was added to the dissolved insoluble fraction, and mixed for 1 h at room temperature. The mixture was loaded into a glass Econo-column, 0.5 cm×5 cm (Bio-Rad, Hercules, Calif.). The liquid phase was flowed through the Ni-NTA resin by gravity and collected as the FT fraction. The column was then washed, sequentially, with 2 mL portions of the lysis buffer containing 6 M, 4 M, 2 M, 1 M, and 0 M urea, respectively. Due to the step-wise decrease of the urea concentration during the wash process, some of the recombinant protein might have been re-natured. Therefore, the protein was eluted from the column using 2 mL of native elution buffer (50 mM NaH₂PO₄, pH 8.0, 300 mM NaCl, 250 mM imidazole) and collected as the native protein fraction. The remaining denatured recombinant protein was eluted from the column using 3 mL of denaturing elution buffer (8 M urea, 0.1 M NaH₂PO₄, 0.01 M Tris-HCl, pH 4.5) and collected as the denatured protein fraction. Both the native and denatured fractions were examined using SDS-PAGE and the results are shown in FIG. 1A, where lanes R and D indicate the re-natured elution fraction and the denatured elution fraction, respectively. These results demonstrate that 6H-TBP1 was purified successfully using the Ni-NTA resin and that some fraction of the protein was re-natured directly on the column (see lanes R and D in FIG. 1A). The protein concentration in each elution fraction was determined using the Bio-Rad protein assay reagent (Bio-Rad Laboratories, Hercules, Calif.). By calculation, we conclude that 1 L of E. coli culture can yield approximately 52 mg of purified 6H-TBP1.

### Example 16

### Preparation of a Sunfast Magenta-Binding Peptide/Peptide Linker/Poly(Ethylene.Terephthalate)-Binding Peptide Triblock Dispersant

The purpose of this Example was to prepare a triblock protein dispersant consisting of a Sunfast Magenta pigment-binding peptide, a hydrophilic peptide linker, and a poly(ethylene terephthalate)-binding peptide using recombinant DNA and molecular cloning techniques.

The designed triblock dispersant has the structure of SM-121 (SEQ ID NO:20)/PT-Linker/PET-121 (SEQ ID NO:40). The PT-Linker is a natural interdomain linker in endoglucanase A of the bacterium Cellulomonas fimi, having the sequence given by SEQ ID NO:56.

Design and Synthesis of TBP2 Gene:

To make the synthetic gene, designated as TBP2, to produce the SM-121 (SEQ ID NO:20)/PT-Linker/PET-121 (SEQ ID NO:40) triblock protein dispersant, the oligonucleotides listed in Table 18 were synthesized by Sigma-Genosys (Woodlands, Tex.).

TABLE 18

Oligonucleotides Used to Prepare the TBP2 Gene

| Oligo | | SEQ |
|---|---|---|
| Name | Sequence (5′-3′) | ID NO: |
| TBP2(+)1 | GGATCCATCGAAGGTCGTCACTCTCTGAAA | 57 |
| | AACTCCATGCTGACTGTTATGGCTGGTGGC | |
| | GGTC | |
| | CGACTCCAACCCCGACTCCGACGCCA | |
| TBP2(+)2 | ACTCCGACTCCGACCCCTACTCCGACGCCG | 58 |
| | ACCCCAACTCCGGGCGGTGGCGGTACCTCT | |
| | GACCACATGATCATGCCGTTCTTCAACTAA | |
| TBP2(−)1 | TTAGTTGAAGAACGGCATGATCATGTGGTC | 59 |
| | AGAGGTACCGCCACCGCCCGGAGTTGGGGT | |
| TBP2(−)2 | CGGCGTCGGAGTAGGGGTCGGAGTCGGAGT | 60 |
| | TGGCGTCGGAGTCGGGGTTGGAGTCGGACC | |
| TBP2(−)3 | GCCACCAGCCATAACAGTCAGCATGGAGTT | 61 |
| | TTTCAGAGAGTGACGACCTTCGATGGATCC | |

Each oligonucleotide was phosphorylated with ATP using T4 polynucleotide kinase. The resulting oligonucleotides were mixed, boiled for 5 min, and then cooled to room temperature slowly. Finally, the annealed oligonucleotides were ligated with T4 DNA ligase to give synthetic DNA fragment TBP2, given as SEQ ID NO:62, which encodes the SM-121/PT-Linker/PET-121 triblock protein dispersant, given as SEQ ID NO:63.

Construction of plNK2 Expression Plasmid:

TBP2 was integrated into the Gateway™ Technology system for protein over-expression (Invitrogen) using the procedure described in Example 15. Primer 5′TBP1 (5′-CAC CGG ATC CAT CGA AGG TCG T-3′), given as SEQ ID NO:64, and primer 3′TBP2 (5′-TCA TTA GTT GAA GAA CGG CAT GA-3′), given as SEQ ID NO:65, were used for amplification of the TBP2 fragment. Due to the design of these primers, an additional sequence of CACC and another stop codon TGA were added to the 5′ and 3′ ends of the amplified fragment. The amplified TBP2 was directly cloned into pENTR/D-TOPO vector using Invitrogen's pENTR directional TOPO cloning kit, resulting in Gateway entry plasmid pENTR-TBP2. This entry plasmid was propagated in the One Shot TOPO10 E. coli strain (Invitrogen). The accuracy of the PCR amplification and cloning were determined by DNA sequencing. Finally, the entry plasmid was mixed with pDEST17. LR recombination reactions were catalyzed by LR Clonase™ (Invitrogen). The destination plasmid, plNK2 was constructed and propagated in the DH5 a E. coli strain. The accuracy of the recombination reaction was determined by DNA sequencing. All reagents for LR recombination reactions were provided in Invitrogen's E. coli expression system with Gateway™ Technology kit. The entire process described above followed Invitrogen's instructions.

The destination plasmid plNK2 contains the coding regions for recombinant protein 6H-TBP2, given as SEQ ID NO:66, which is a 10.7 kDa protein. This protein sequence includes a 6×His tag and a Factor Xa recognition site that is identical to that of 6H-TBP1, as described in Example 15. Following this N-terminal sequence, 6H-TBP2 contains a tri-block protein TBP2, which has the structure of SM-121/PT-Linker/PET-121. TBP2 can be released from 6H-TBP2 by Factor Xa treatment.

Production of 6H-TBP2 Protein:

The expression plasmid plNK2 was transformed into the BL21-Al E. coli strain (Invitrogen) using the procedure described in Example 15 for the production of 6H-TBP2 protein.

Aliquots (13 μL) of the soluble fraction and the dissolved insoluble fraction were subjected to 10% SDS-PAGE and the resulting gel was stained with Coomassie brilliant blue G-250. The electrophoresis results are shown in FIG. 1B, where lanes I and S indicate the insoluble fraction and the soluble fraction, respectively. These results indicate that the protein 6H-TBP2 was expressed in the insoluble fraction (see lanes S and I in FIG. 1B).

The recombinant protein was purified using Ni-NTA Superflow resin (QIAGEN), as described in Example 15. Both the native and denatured fractions were examined using SDS-PAGE and the results are shown in FIG. 1B, where lanes R and D indicate the re-natured elution fraction and the denatured elution fraction, respectively. These results demonstrate that 6H-TBP2 was purified successfully using the Ni-NTA resin and that some fraction of the protein was re-natured directly on the column (see lanes R and D in FIG. 1B). The protein concentration in each elution fraction was determined using the Bio-Rad protein assay reagent (Bio-Rad Laboratories, Hercules, Calif.). By calculation, we conclude that 1 L of E. coli culture can yield approximately 40 mg of purified 6H-TPB2.

### Example 17

### Characterization of the 6H-TBP1 and 6H-TBP2 Proteins

The 6H-TBP1 (SEQ ID NO: 55) and 6H-TBP2 (SEQ ID NO:66) recombinant proteins in the denatured fractions were re-natured by dialysis in lysis buffer overnight at 4° C. During the course of dialysis, the urea concentration in the buffer was decreased to 0 gradually. The proteins that failed to re-nature and formed a precipitate were removed by centrifugation. Finally, all re-natured recombinant proteins were combined and dialyzed in the INK buffer (50 mM Tris-HCl, pH 7.5, 50 mM NaCl, 1 mM β-mercaptoethanol, 20% glycerol).

The binding affinity of these proteins was determined using the ELISA-based binding assay, which is described in Example 4. Instead of phage particles, 1 μg of 6H-TBP1 or 6H-TBP2 was used in the assay. The protein solutions were centrifuged just prior to the analysis to remove any precipitated protein. Five μg of a 42 kDa, C-terminal 6×His-tagged recombinant protein (Ctrl-6H), which is the translation product of the E. coli YqhD gene, was used as a control. Any C-terminal-His tagged recombinant protein of similar molecular weight may be used as the control. The proteins that interacted with the substrate were detected using a 1:2,000 dilution of Penta-His Ab (QIAGEN) as the first antibody and a 1:1,000 dilution of Goat Anti-Mouse IgG-HRP conjugate as the second antibody. The results of the ELISA assays, given as the mean and standard deviation of triplicate determinations run in parallel, are summarized in Table 19 As can be seen from the results in the table, both the TBP1 and TBP2 triblock protein dispersants specifically bind to their respective substrates, although the binding affinity of TBP1 for its substrates is higher than the binding affinity of TBP2 for its substrates.

TABLE 19
Results of ELISA-Based Binding Assay
for 6H-TBP1 and 6H-TBP2

| Substrate | Protein | OD₄₀₅ |
|---|---|---|
| Carbon Black | 6H-TBP1 | 0.350 ± 0.065 |
| | Ctrl-6H | 0.010 ± 0.003 |
| Sigmacell cellulose | 6H-TBP1 | 0.471 ± 0.004 |
| | Ctrl-6H | 0.005 ± 0.003 |
| Sunfast ® Magenta | 6H-TBP2 | 0.061 ± 0.012 |
| | Ctrl-6H | 0.002 ± 0.007 |
| PET particles | 6H-TBP2 | 0.046 ± 0.006 |
| | Ctrl-6H | 0.011 0.002 |

### Example 18

### Performance Testing of a Triblock Peptide Dispersant

The purpose of this prophetic Example is to describe the testing of a triblock peptide dispersant. The triblock dispersant consists of a carbon black-binding peptide (SEQ ID NO:6), the ST-Linker (SEQ ID NO:45) and a cellulose-binding peptide (SEQ ID NO:37).

Carbon Black Ink Formulation:

The triblock peptide dispersant, given as SEQ ID NO:52, is prepared as described in Example 15 and is used in all the formulations in this Example. Carbon black ink dispersions are prepared using the bottle shake method described in Example 9. Details of the three different ink formulations are described in Table 20. The dispersions are made using Printex 150T Carbon Black Pigment (Degussa, Piscataway, N.J.). Sand (0.5 g) is used as the abrasive medium.

TABLE 20
Carbon Black Ink Dispersion Formulations

| | Dispersant | Pigment | Defoamer | 10% KOH | |
|---|---|---|---|---|---|
| Formulation | (g) | (g) | (g) | (g) | Water (g) |
| 1 | 0.25 | 0.375 | 0.1 | 0.066 | 10.0 |
| 2 | 0.25 | 0.375 | 0.1 | 0.128 | 10.0 |
| 3 | 0.25 | 0.375 | 0.1 | 0.023 | 10.0 |

Dispersion Testing of Triblock Peptide Dispersant:

The dispersion testing is done as described in Example 9. Formulations 1 and 2 are predicted to give good dispersions, while formulation 3 is predicted to give a good to very good dispersion.

### Example 19

Preparation of a Soluble Triblock Protein Dispersant

In Examples 15 and 16, a method for preparing triblock protein dispersants was demonstrated. The purpose of this Example was to demonstrate an improved method for preparing triblock protein dispersants. In the improved method, the construct pENTR-TBP1 was modified to add appropriate restriction sites between each element of its triblock protein-coding region. This modification allows substitution of each element by a simple “cut-and-paste” method, so that the construct can be applied to prepare and examine any combination of peptide and linker candidates. Additionally, GST (glutathione S-transferase) protein was used as a fusion partner to produce and purify the water-soluble triblock protein dispersant.

Modification of pENTR-TBP1:

The plasmid pENTR-TBP1, described in Example 15, was subjected to site directed mutagenesis using the Quikchange kit (Strategene, La Jolla, Calif.) to add a unique NgoMl site (GCCGGC) at the beginning and a Kasl site (GGCGCC) at the end of the linker coding sequence, so that each element within the TBP1 coding region on this plasmid can be replaced by others through unique restriction digestion. This procedure resulted in Gateway entrance plasmid pENTR-TBP101, which has a coding sequence, given as SEQ ID NO:116, that encodes for IEGR (SEQ ID NO:117)/CB-72 (SEQ ID NO:6)::ST-Linker (SEQ ID NO:45)::CEL-121 (SEQ ID NO:37). IEGR represents an Xa recognition site. The corresponding amino acid sequence for the encoded protein is given as SEQ ID NO:118.

In addition, the ST linker sequence in pENTR-TBP101 was replaced by the PT linker (SEQ ID NO:56). Due to its small size, the PT linker sequence could not be cut and pasted directly. Instead, the linker coding sequence and the flanking regions were amplified from pENTR-TBP2 (see Example 16) by PCR reaction and digested with NgoMl and Kasl. The resulting fragment was inserted into pENTR-TBP101 to replace the previous linker sequence. This insertion formed Gateway entrance plasmid pENTR-TBP201, which has the coding sequence, given as SEQ ID NO:119, that encodes for IEGR (SEQ ID NO:117)/CB-72 (SEQ ID NO:6)::PT-Linker (SEQ ID NO:56)::CEL-121 (SEQ ID NO:37). The corresponding amino acid sequence for the encoded protein is given as SEQ ID NO:120.

The TBP structure in the modified entrance plasmid pENTR-TBP201 was introduced into the Gateway E. coli expression system (Invitrogen) by taking advantage of the site-specific recombination of bacteriophage lambda, described in Examples 15 and 16. Briefly, the recombination was conducted in a 20-μL reaction, containing 0.3 μg of the entrance plasmid DNA, 0.3 μg of pDEST15 destination plasmid DNA, 4 μL of colonase buffer, and 4 μL of LR colonase mixture, for 1 h at 25° C. Then, 4 μL of Proteinase K was added and the reaction mixture was heated at 37° C. for 10 min. The resulting reaction mixture (2 μL) was used to transform 50 μL of Library Efficient DH5α competent cells. The transformed cells were recovered in 500 μL of SOC medium and grown on LB-Amp50 plates. E. coli colonies on the plates were selected and further grown in LB-Amp broth. Expression plasmid DNA was isolated from the cells using a QIAprep Miniprep Kit (Qiagen, Valencia, Calif). The construct was confirmed by DNA sequencing. The reaction generated the expression plasmid plNK251, given as SEQ ID NO:121. This plasmid encoded a GST-TBP251 (GST-CB72::PT-Linker::CEL-121) fusion protein, given as SEQ ID NO:122.

Expression and Biochemical Characterization of GST-TBP251 Fusion Protein:

The expression plasmid plNK251 was expressed and the GST-TBP251 fusion protein was purified in small scale in order to perform a micro-scale functional analysis. To express plNK251, the plasmid was transformed into BL21 A1 E. coli cells (Invitrogen). One transformed colony was grown in 10 mL of LB-Amp50 broth overnight at 37° C. and then re-grown in larger scale by inoculating a 20× volume of fresh LB-Amp50 with the overnight-grown cells. When the cell density reached an OD₆₀₀ value of 0.8 to 1.0, L-arabinose was added to the culture to a concentration of 0.2% and protein expression was induced for 4 h at 37° C. Approximately 100 μL of cells was spun down and lysed in 30 μL of SDS-PAGE loading buffer by boiling for 10 min. Expression of the GST-TBP251 fusion protein was confirmed by running 10 μL of cell lysate on a NuPAGE® SDS-PAGE gel (Invitrogen).

To purify the expressed protein, E. coli cells collected from 200 mL of culture were lysed in 5 mL of CelLytic B bacterial cell lysis extraction reagent (Sigma Chemical Company), containing 15 μg DNase I and 100 μL of proteinase inhibitor cocktail (Sigma Chemical Company), for 30 min at room temperature. Soluble and insoluble fractions were separated by centrifugation. SDS-PAGE loading buffer was added to portions of both fractions and their protein profiles were examined by SDS-PAGE. The results showed that the GST-TBP251 fusion protein was fully soluble. The lysate was loaded onto a 1.5 mL Glutathione-Agarose column (Sigma Chemical Company), and the column was washed with 10 mL of TBS containing 1% Tween-20. The GST-TBP251 fusion protein was eluted from the column 4 times using 2 mL portions of FST elution buffer (50 mM Tris-HCl, pH 9.5, and 10 mM reduced glutathione). The protein profile in each purification step was examined by SDS-PAGE, the results of which confirmed the presence of the 33.7 kDa GST-TBP251 fusion protein in the eluted fraction. The purified protein was dialyzed against IEB buffer and the protein concentration was determined using the Bio-Rad protein assay reagent.

Binding Activity of GST-TBP251 Fusion Protein:

The binding activity of GST-TBP251 fusion protein to carbon black and cellulose was characterized by performing the ELISA-based binding assay described in Example 6. For the determination of carbon black pigment-binding activity, 75 μg of Carbon Black FW-18 (Degussa) was used while 150 μg of Sigmacell (Cellulose Type 20, Sigma) was used for the determination of cellulose-binding activity. In the assay, reactions without any protein present were used as the blank reactions, and reactions containing 4 μg (approximately 0.1 nmol) of GST served as a negative control. The ELISA results are presented in Table 21. The results confirm that the GST-TBP251 fusion protein possessed binding activity for both carbon black and cellulose.

TABLE 21
Results of ELISA-Based Binding Assay for GST-TBP251

| Substrate | Protein | OD₄₀₅ |
|---|---|---|
| Carbon black | GST-TBP251 | 0.677 ± 0.053 |
| Carbon black | GST | 0.110 ± 0.045 |
| Sigmacell | GST-TBP251 | 0.184 ± 0.023 |
| Sigmacell | GST | 0.011 ± 0.002 |

### Example 20

Performance Testing of the GST- TBP251 Fusion Protein The dual binding activities of GST-TBP251 fusion protein were demonstrated (see Example 19); however, that result does not necessarily demonstrate that the protein can function as either a pigment dispersant or a print medium binder. Therefore, the purpose of this Example was to demonstrate that the TBP251 protein functions effectively as a dispersant for carbon black and that it binds to cellulose.

Preparation of Triblock Protein TBP251:

The TBP251 protein was cleaved from the GST-TBP251 fusion protein as follows. In the cleavage reaction, 100 μg of biotin-labeled restriction protease factor Xa (Roche, Mannheim, Germany) was added to a protein solution containing 10 mg of GST-TBP251 fusion protein and the reaction mixture was incubated for 18 h at 4° C. The reaction resulted in a 4.87 kDa TBP251 protein and a 28.9 kDa GST fusion partner, along with the biotin-labeled factor Xa and little non-cleaved GST-TBP251 protein in the mixture, as determined by SDS-PAGE. The TBP251 protein was purified from the reaction mixture by passing the mixture through a glutathione-agarose column twice, followed by molecular filtration using a Centricon®-3,000 Centrifugal Filter Device (Millipore Corp., Billerica, Mass.).

Micro-Scale Assay of Carbon Black Pigment Dispersion by TBP251:

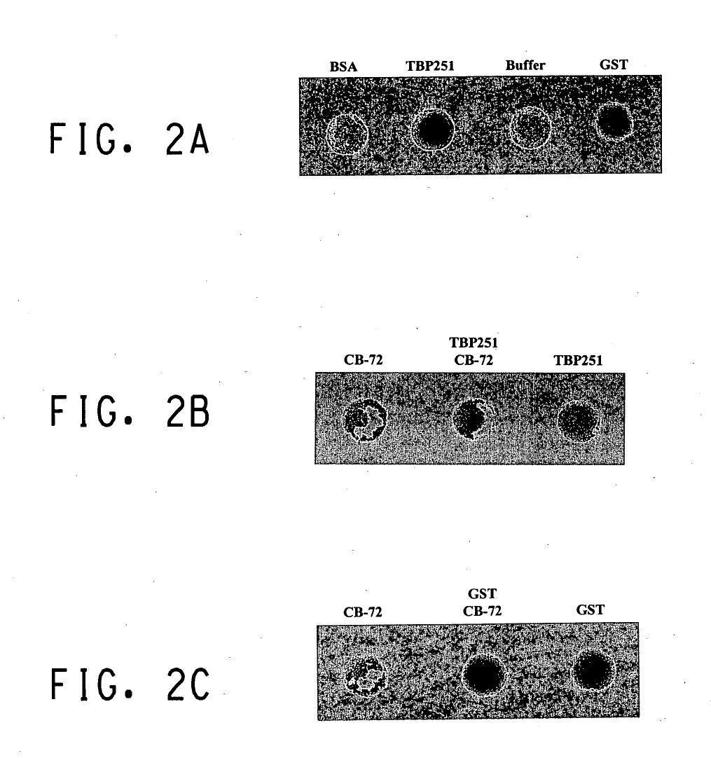

To demonstrate that the TBP251 protein stabilizes the dispersion of carbon black pigment, a micro-scale dispersion assay was developed, which mimics the standard dispersion test. In this assay, a 10-μL dispersion reaction mixture was prepared, which included 50 μg of Carbon Black FW-18 (Degussa) and 3 μg (0.6 nmol) of TBP251 protein in 10 μL of IEB buffer. After quick and gentle mixing, 5 μL of the mixture was dropped on a piece of glass micro-slide (VWR Scientific, Media, Pa.). The glass slide was left undisturbed for 2 min, and then observed for pigment dispersion. The results are shown in FIG. 2A. As shown in the figure, the carbon black pigment was finely dispersed when the TBP251 protein was added. For comparison, the dispersion test was also conducted using 42 μg (0.6 nmol) of BSA and with buffer (no protein). As shown in FIG. 2A, both of these mixtures exhibited flocculation of the carbon black pigment. Since the dispersion demonstrated in this Example does not require strong physical force, the interaction between TBP251 and carbon black appears strong enough to mediate the self-dispersion of the pigment. This is a property that the current state-of-the-art copolymer-based dispersants do not offer.

Dispersion testing was also done using 18 μg (0.6 nmol) of GST in place of TBP251 in the dispersion mixture. As shown in FIG. 2A, GST functioned as a carbon black pigment dispersant as well. Since GST is a natural protein that does not contain a carbon black binding peptide or binding domain, it was proposed that the dispersion function of GST has a mechanism different from that of TBP251. To demonstrate this, a competition test of carbon black dispersion was conducted. Compared to the dispersion test described above, the competition mixture contained no glycerol and much less protein in order to make the pigment dispersion easier to be reversed. Therefore, 60 μg of Carbon Black FW-18 (Degussa) was gently mixed with 13 μL of IEB buffer (containing no glycerol) that contained 0.42 μg (0.1 nmol) of TBP251 protein. The mixture (5 μL) was dropped onto a piece of glass micro-slide, which was left undisturbed for 2 min, and then observed for pigment dispersion. The result indicated that the reduced amount of TBP251 protein was sufficient to disperse the carbon black pigment in the mixture. When 0.6 μg (0.66 nmol) of synthetic CB-72 peptide (SEQ ID NO:6 ), obtained from SynPep (Dublin, Calif.), was also included in the mixture, the excess amount of the competitor peptide blocked the dispersion function of TBP251, resulting in flocculation of the pigment in the mixture (FIG. 2B). The same level of flocculation was also observed in a negative control mixture that contained 0.6 μg (0.66 nmol) of synthetic CB-72 peptide but lacked TBP251 protein (FIG. 2B). These competition results demonstrated that the carbon black-binding activity of the CB-72 peptide sequence is directly involved in the pigment dispersion mechanism of TBP251 protein. When the same competition test was conducted using 2.52 μg (0.1 nmol) of GST instead of TBP251, the CB-72 peptide failed to block the dispersion function of GST (FIG. 2C). This result suggested that the pigment dispersion by GST does not depend on the specific activity of CB-72 peptide, and thus has a different mechanism. Due to the lack of glycerol in the competition test mixtures, flocculation of the carbon black pigment was much greater (FIG. 2B and 2C) than in the initial dispersion testing (FIG. 2A).

Micro-Scale Assay of Pigment Adhesion to Print Medium Surface Mediated by TBP251:

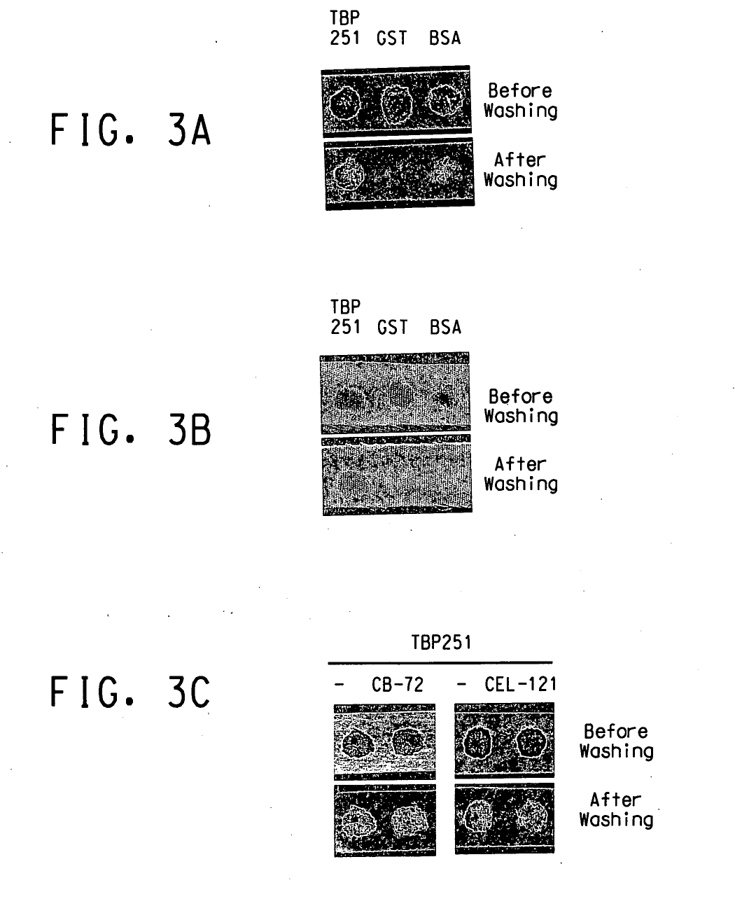

To demonstrate that TBP251 protein can attach carbon black pigment to the surface of a print medium, a micro-scale assay was developed, which mimics the standard washfastness durability test. In this assay, 200 μg of Carbon Black FW-18 (Degussa) was added to 40 μL of IEB buffer containing 12 μg (2.5 nmol) of TBP251 protein. In two negative control mixtures, 72 μg (2.5 nmol) of GST and 168 μg (2.5 nmol) of BSA were used in place of TBP251. After a 20 min incubation at room temperature, 5 μL of the mixture was dropped on a piece of Whatman 5 filter paper (Whatman, Clifton, N.J.). Due to its excellent water absorbance capability, the paper absorbed water quickly and left carbon black on the surface to allow direct interaction between the paper surface and the pigment-protein complex. The paper was then placed in 10 mL of TBST and washed for 2 min with vigorous shaking. The results showed that carbon black attached to the paper surface when the mixture included TBP251, but not when GST or BSA was used, as shown in FIG. 3A. The same results were obtained when the test was repeated using 100% cotton fabric as the print medium (FIG. 3B). Although the signals were weaker because the large space between fibers on the cotton fabric allowed a lot of the pigment to go through, rather than keeping it on the surface, it was clear that TBP251 enhanced the attachment of the pigment to the surface of the cotton fabric, but GST and BSA did not. Therefore, TBP251 indeed functions as a print medium binder to attach carbon black pigment to fabrics that contain cellulose.

According to the design of the TBP251 protein, the CB-72 sequence interacts with carbon black and the CEL-121 sequence interacts with cellulose-containing print media. Both carbon black- and cellulose-binding activities contribute to TBP251's function as a binder. To directly demonstrate this, a competition test for paper attachment was developed. In the test with a carbon black binding competitor, 50 μg of Carbon Black FW-18 (Degussa) was mixed with 5 μL of IEB buffer containing 7.5 μg (8.2 nmol) of synthetic CB-72 peptide for 10 min at room temperature. Then, 10 μL of IEB buffer containing 3 μg (0.61 nmol) of TBP251 protein was added to the mixture. After a 20 min incubation at room temperature, 5 μL of the mixture was dropped on a piece of Whatman 5 filter paper. The paper was placed in 10 mL of TBST and washed for 2 min with vigorous shaking. The attachment of carbon black to the paper was observed before and after washing.

In the test with a print medium binding competitor, the carbon black was mixed with both 11 μg (8.2 nmol) of synthetic CEL-121 peptide and 3 μg (0.61 nmol) of TBP251 protein at same time, rather than following a specific order, in 15 μL of IEB buffer.

The results of both competition tests are shown in FIG. 3C. The mixtures without competitors served as positive controls. These results demonstrate that free CB-72 and CEL-121 peptides can block, at least partially, TBP251's function as a print medium binder for carbon black pigment. Therefore, the TBP251 protein possesses carbon black- and cellulose-binding activities and functions to disperse carbon black pigment and to attach the pigment to cellulose-containing print media.

1. A pigment-binding peptide selected from the group consisting of SEQ ID NO:7, 8, 9, 10, 14, 15, 16, 17, 18, 19, 20, 21, 23, 24, 25, 26, 27, 28, and 29.

2. A print medium-binding peptide selected from the group consisting of SEQ ID NO: 31, 32, 34, 35, 36, 37, 38, and 40.

3. A polyester-binding peptide selected by a process comprising the steps of:
a) providing a library of combinatorial generated peptides;
b) contacting the library of (a) with a pigment to form a reaction solution comprising:
(i) peptide-polyester complexes;
(ii) unbound polyester; and
(iii) uncomplexed peptides;
c) isolating the peptide-polyester complexes of (b); and
d) eluting the peptides from the peptide complex of (b), wherein the eluted peptide has specific polyester binding properties.

4. A polyester binding peptide according to claim 3 wherein the peptide is from about 5 to about 20 amino acids, comprises at least about 20 mole % of amino acids selected from the group consisting of phenylalanine, tryptophan, and tyrosine, and has a binding energy of at least 20 kcal/mol.

5. A polyester-binding peptide having from about 5 to about 20 amino acids, comprising at least about 20 mole % of amino acids selected from the group consisting of phenylalanine, tryptophan, and tyrosine, and having a binding energy to a polyester substrate equal to or greater than 20 kcal/mol.

6. A diblock dispersant having the general structure: [PBP]ₙ—HL, wherein
a) PBP is a pigment-binding peptide;
b) HL is a hydrophilic linker; and
c) n ranges from 1 to about 5.

7. A diblock dispersant according to claim 6 wherein wherein the pigment binding peptide is from about 5 amino acids to about 20 amino acids, comprises at least about 40 mol % of an amino acid selected from the group consisting of glycine, alanine, valine, leucine, isoleucine, methionine, proline, phenylalanine, and tryptophan, and has a binding energy of at least 20 kcal/mol.

8. A diblock dispersant according to claim 6 wherein the pigment binding peptide is selected by a process of comprising:
(i) providing a library of combinatorial generated peptides;
(ii) contacting the library of (i) with a pigment to form a reaction solution comprising:
(A) peptide-pigment complexes;
(B) unbound pigment; and
(C) uncomplexed peptides;
(iii) isolating the peptide-pigment complexes of (ii); and
(iv) eluting the peptides from the peptide complex of (ii).

9. A diblock polymer having the general structure: [PMBP]ₙ—HL, wherein
a) PMBP is a print medium-binding peptide;
b) HL is a hydrophilic linker; and
c) n ranges from 1 to about 5.

10. A diblock polymer according to claim 9 wherein the print medium-binding peptide is selected from the group consisting of cellulose-binding peptide and a polyester-binding peptide.

11. A diblock polymer according to claim 10 wherein the cellulose-binding peptide is about 5 amino acids to about 20 amino acids, comprises at least about 14 mol % of an amino acid selected from the group consisting of serine, threonine, and tyrosine, and has a binding energy of at least 20 kcal/mol.

12. A diblock polymer according to claim 10 wherein the polyester-binding peptide is from about 5 amino acids to about 20 amino acids, comprises at least about 20 mol % of an amino acid selected from the group consisting of phenylalanine, tryptophan, and tyrosine, and has a binding energy of at least 20 kcal/mol.

13. A diblock polymer according to claim 10 wherein the cellulose-binding peptide is selected by a process comprising:
(i) providing a library of combinatorial generated peptides;
(ii) contacting the library of (i) with cellulose to form a reaction solution comprising:
(A) peptide-cellulose;
(B) unbound cellulose; and
(C) uncomplexed peptides;
(iii) isolating the peptide-cellulose complexes of (ii); and
(iv) eluting the peptides from the peptide complex of (iii).

14. A diblock polymer according to claim 10 wherein the polyester-binding peptide is selected by a process comprising:
(i) providing a library of combinatorial generated peptides;
(ii) contacting the library of (i) with polyester to form a reaction solution comprising:
(A) peptide-polyester;
(B) unbound polyester; and
(C) uncomplexed peptides;
(iii) isolating the peptide-polyester complexes of (ii); and
(iv) eluting the peptides from the peptide complex of (iii).

15. A diblock dispersant having the general structure: [PBP]ₙ—[PMBP]ₙ, wherein
a) PBP is a pigment-binding peptide;
b) PMBP is a print medium-binding peptide; and
c) n ranges from 1 to about 5.

16. A diblock dispersant according to claim 15 wherein the pigment-binding peptide is from about 5 amino acids to about 20 amino acids, comprises at least about 40 mol % of an amino acid selected from the group consisting of glycine, alanine, valine, leucine, isoleucine, methionine, proline, phenylalanine, and tryptophan, and has a binding energy of at least 20 kcal/mol.

17. A diblock dispersant according to claim 15 wherein print medium-binding peptide is from about 5 amino acids to about 20 amino acids, comprises at least about 14 mol % of an amino acid selected from the group consisting of serine, threonine, and tyrosine, and has a binding energy of at least 20 kcal/mol, comprises at least about 20 mol % of an amino acid selected from the group consisting of phenylalanine, tryptophan, and tyrosine, and has a binding energy of at least 20 kcal/mol.

18. A diblock dispersant according to claim 15 wherein the pigment-binding peptide is selected by a process comprising the steps of:
(i) providing a library of combinatorial generated peptides;
(ii) contacting the library of (i) with a pigment to form a reaction solution comprising:
(A) peptide-pigment complexes;
(B) unbound pigment; and
(C) uncomplexed peptides;
(iii) isolating the peptide-pigment complexes complexes of (ii); and
(iv) eluting the peptides from the peptide complex of (ii).

19. A diblock dispersant according to claim 15 wherein the print medium-binding peptide is selected by a process comprising the steps of:
(i) providing a library of combinatorial generated peptides;
(ii) contacting the library of (i) with cellulose or polyester to form a reaction solution comprising:
(A) peptide-cellulose complexes or peptide-polyester-complexes;
(B) unbound cellulose or polyester; and
(C) uncomplexed peptides;
(iii) isoating the peptide-cellulose or peptide-polyester complexes of (ii); and
(iv) eluting the peptides from the peptide complex of (ii).

20. A diblock dispersant having the general structure: [PMBP]ₙ—HPL, wherein,
a) PMBP is a print medium-binding peptide;
b) HPL is a hydrophobic linker; and
c) n ranges from 1 to about 5.

21. A diblock dispersant according to claim 20 wherein the print medium-binding peptide is selected from the group consisting of cellulose-binding peptide and a polyester-binding peptide.

22. A diblock dispersant according to claim 21 wherein the cellulose-binding peptide is about 5 amino acids to about 20 amino acids, comprises at least about 14 mol % of an amino acid selected from the group consisting of serine, threonine, and tyrosine, and has a binding energy of at least 20 kcal/mol.

23. A diblock dispersant according to claim 21 wherein the polyester-binding peptide is from about 5 amino acids to about 20 amino acids, comprises at least about 20 mol % of an amino acid selected from the group consisting of phenylalanine, tryptophan, and tyrosine, and has a binding energy of at least 20 kcal/mol.

24. A diblock dispersant according to claim 22 wherein the cellulose-binding peptide is selected by a process comprising:
(i) providing a library of combinatorial generated peptides;
(ii) contacting the library of (i) with cellulose to form a reaction solution comprising:
(A) peptide-cellulose;
(B) unbound cellulose; and
(C) uncomplexed peptides;
(iii) isolating the peptide-cellulose complexes of (ii); and
(iv) eluting the peptides from the peptide complex of (iii).

25. A diblock dispersant according to claim 23 wherein the polyester-binding peptide is selected by a process comprising:
(i) providing a library of combinatorial generated peptides;
(ii) contacting the library of (i) with polyester to form a reaction solution comprising:
(A) peptide-polyester;
(B) unbound polyester; and
(C) uncomplexed peptides;
(iii) isolating the peptide- polyester complexes of (ii); and
(iv) eluting the peptides from the peptide complex of (iii).

26. A diblock polymer having the general structure: [PMBP]ₙ—BA, wherein,
a) PMBP is a print medium-binding peptide selected from the group consisting of SEQ ID NO: 4, 30, 31, 32, 33, 34, 35, 36, 37, 38, and 40;
b) BA is a benefit agent; and
c) n ranges from 1 to about 5.

27. The diblock polymer of claim 26 wherein the benefit agent is selected from the group consisting of perfumes, hygiene agents, insect control agents, enzymes, softening protein, fabric softening agents, soil release agents, bleaching agents, dye fixative agents, brighteners, antimicrobial agents, surfactants and mixtures thereof.

28. A triblock dispersant having the general structure: [PBP]ₙ—HL—[PMBP]ₙ, wherein
a) PBP is a pigment-binding peptide;
b) HL is a hydrophilic linker;
c) PMBP is a print medium-binding peptide; and
d) n ranges from 1 to about 5.

29. A triblock dispersant according to claim 28 wherein the pigment-binding peptide is from about 5 amino acids to about 20 amino acids, comprises at least about 14 mol % of an amino acid selected from the group consisting of serine, threonine, and tyrosine, and has a binding energy of at least 20 kcal/mol,comprises at least about 20 mol % of an amino acid selected from the group consisting of phenylalanine, tryptophan, and tyrosine, and has a binding energy of at least 20 kcal/mol.

30. A triblock dispersant according to claim 28 wherein the print medium-binding peptide is selected from the group consisting of cellulose-binding peptide and a polyester-binding peptide.

31. A triblock dispersant according to claim 30 wherein the cellulose-binding peptide is about 5 amino acids to about 20 amino acids, comprises at least about 14 mol % of an amino acid selected from the group consisting of serine, threonine, and tyrosine, and has a binding energy of at least 20 kcal/mol.

32. A triblock dispersant according to claim 30 wherein the polyester-binding peptide is from about 5 amino acids to about 20 amino acids, comprises at least about 20 mol % of an amino acid selected from the group consisting of phenylalanine, tryptophan, and tyrosine, and has a binding energy of at least 20 kcal/mol.

33. A triblock dispersant according to claim 31 wherein the cellulose-binding peptide is selected by a process comprising:
(i) providing a library of combinatorial generated peptides;
(ii) contacting the library of (i) with cellulose to form a reaction solution comprising:
(A) peptide-cellulose;
(B) unbound cellulose; and
(C) uncomplexed peptides;
(iii) isolating the peptide-cellulose complexes of (ii); and
(iv) eluting the peptides from the peptide complex of (iii).

34. A triblock dispersant according to claim 32 wherein the polyester-binding peptide is selected by a process comprising:
(i) providing a library of combinatorial generated peptides;
(ii) contacting the library of (i) with polyester to form a reaction solution comprising:
(A) peptide-polyester;
(B) unbound polyester; and
(C) uncomplexed peptides;
(iii) isolating the peptide-polyester complexes of (ii); and
(iv) eluting the peptides from the peptide complex of (iii).

35. The dispersant or polymer of claims 6, 15 or 28, wherein the pigment-binding peptide or the print medium-binding peptide is from about 7 amino acids to about 12 amino acids

36. The dispersant or polymer of claims 6, 15, or 28, wherein the pigment-binding peptide has the amino acid sequence selected from the group consisting of SEQ ID NO:5, 6, 7, 8, 9, 10, 14, 15, 16, 17, 18, 19, 20, 21, 23, 24, 25, 26, 27, 28, and 29.

37. The dispersant or diblock polymer of claims 9, 15, or 20, wherein the print medium binding peptide has the amino acid sequence selected from the group consisting of SEQ ID NO:4, 30, 31, 32, 33, 34, 35, 36, 37, 38, and 40.

38. The triblock dispersant of claim 28, wherein the print medium-binding peptide has the amino acid sequence selected from the group consisting of SEQ ID NO:1-4, 30, 31, 32, 33, 34, 35, 36, 37, 38, and 40.

39. The dispersant or polymer of claims 6, 9 or 28, wherein the hydrophilic linker is an organic polymer selected from the group consisting of polyethylene glycol derivatives, polyacrylic acid/maleic acid polymers, derivatives of polyacrylic acid, polyvinyl alcohol, polyacrylamide, polymethylvinyl ether, cellulosic polymers and other polysaccharides.

40. The dispersant or polymer of claims 6, 9 or 28, wherein the hydrophilic linker is a peptide comprising amino acids selected from the group consisting of aspartic acid, asparagine, glutamic acid, glutamine, histidine, arginine, lysine, proline, serine, threonine, and mixtures thereof.

41. The dispersant or polymer of claim 40 wherein the hydrophilic linker is a peptide having a length of 3 to about 50 amino acids.

42. The dispersant or polymer of claim 40 wherein the hydrophilic linker is a peptide selected from the group consisting of polyproline, SEQ ID NO:45, and SEQ ID NO:56.

43. The diblock dispersant of claim 20 wherein the hydrophobic linker is a synthetic organic polymer selected from the group consisting of methacrylate, butyl methacrylate, 2-ethylhexyl methacrylate, benzylmethacrylate, and copolymers thereof.

44. The diblock dispersant of claim 20 wherein the hydrophobic linker is a peptide comprising amino acids selected from the group consisting of alanine, glycine, methionine, leucine, isoleucine, valine, cysteine, phenylalanine, tyrosine, tryptophan, hydrophobic derivatives of hydrophilic amino acids, and mixtures thereof.

45. The dispersant or polymer of claims 6, 15, or 28, wherein the pigment-binding peptide has affinity for organic pigments selected from the group consisting of Cyan, Yellow, Red, Blue, Orange, Magenta, Black, Green, Violet, Light Cyan, and Light Magenta.

46. The dispersant or polymer of claim 45 wherein the pigment-binding peptide has affinity for organic pigments selected from the group consisting of carbon black, Cromophthal® Yellow, Sunfast® Magenta and Sunfast® Blue.

47. The dispersant or diblock polymer of claim 9, 15, or 20, wherein the print medium-binding peptide has affinity for paper and textile fabrics selected from the group consisting of cotton, polyester/cotton, cellulose, and poly(ethylene terephthalate).

48. The dispersant or polymer of claims 6, 15, or 28, wherein the pigment-binding peptide or the print medium-binding peptide has a binding energy equal to or greater than 20 kcal/mol.

49. An aqueous ink composition comprising:
a) an aqueous carrier medium;
b) a pigment; and
c) the dispersant of claim 6, 15, or 28.

50. The ink composition according to claim 49 wherein the composition contains about 0.1 to about 15% pigment, about 0.1 to about 30% dispersant, and about 70 to about 99.8% aqueous carrier medium.

51. The ink composition according to claim 49 wherein the aqueous medium comprises water or a mixture of water and at least one organic solvent.

52. The ink composition according to claim 51 wherein the organic solvent is a polyhydric alcohol.

53. The ink composition according to claim 51 further comprising a surfactant.

54. The ink composition according to claim 53 wherein the surfactant is selected from the group consisting of anionic, nonionic, cationic, and amphoteric surfactants.

55. The ink composition according to claim 53 wherein the surfactant is present in the amount of about 0.01 to about 5%.

56. The ink composition according to claim 53 wherein the surfactant is present in the amount of about 0.2 to about 2%.

57. A water-born coating composition comprising:
a) an aqueous carrier medium;
b) a pigment;
c) a film-forming resin; and
d) the dispersant or polymer of claims 6, 15, or 28.

58. A pigmented plastic composition comprising:
a) a plastic resin;
b) a pigment; and
c) the dispersant or polymer of claims 6, 15, 28.

59. A method for recovering and purifying a recombinant protein comprising:
a) providing a sample comprising a recombinant protein containing a cellulose-binding peptide sequence selected from the group consisting of SEQ ID NO:35, 36, 37, and 38;
b) contacting the sample with a cellulose support;
c) separating the cellulose support from the sample; and
d) recovering the recombinant protein by treating the support with an eluting agent.

60. A method for recovering and purifying a recombinant protein comprising:
a) providing a sample comprising a recombinant protein containing a pigment-binding peptide sequence selected from the group consisting of SEQ ID NO:5, 6, 7, 8, 9, 10, 14, 15, 16, 17, 18, 19, 20, 21, 23, 24, 25, 26, 27, 28, and 29;
b) contacting the sample with a pigment;
c) separating the pigment from the sample; and
d) recovering the recombinant protein by treating the pigment with an eluting agent.

61. A method for affixing a pigment to a print medium comprising:
a) providing a triblock dispersant according to claim 28 having a pigment-binding peptide and a print-media-binding-peptide;
b) contacting the triblock dispersant of (a) with a pigment wherein the pigment complexes with the pigment-binding-peptide to form a peptide complexed triblock: and
c) contacting the peptide complexed triblock of step (c) with a print-medium wherein the print-medium complexes with the print-media-binding-peptides and the pigment is affixed to the print medium.

62. A method according to claim 61 wherein steps b) and c) are reversed.

63. A method according to claim 61 wherein the triblock dispersant is provided as part of a composition selected from the group consisting of: an ink composition, water-born coating composition and a pigmented plastic composition.


SEQUENCE LISTING (122 sequences)

SEQ ID NO: 1 - protein, 10 aa, Artificial Sequence
  Mimi CBD Peptide
AWAWAWAWKK

SEQ ID NO: 2 - protein, 10 aa, Artificial Sequence
  Mimic CBD Peptide
WEWEWEWEKK

SEQ ID NO: 3 - protein, 4 aa, Artificial Sequence
  Mimic CBD Peptide
SWYL

SEQ ID NO: 4 - protein, 7 aa, Artificial Sequence
  Paper Binding Peptide
HAIYPRH

SEQ ID NO: 5 - protein, 12 aa, Artificial Sequence
  Pigment Binding Peptide
SVSVGMKPSPRP

SEQ ID NO: 6 - protein, 7 aa, Artificial Sequence
  Pigment Binding and Cellulose Binding Peptide
FHENWPS

SEQ ID NO: 7 - protein, 7 aa, Artificial Sequence
  Pigment Binding Peptide
MPPPLMQ

SEQ ID NO: 8 - protein, 12 aa, Artificial Sequence
  Pigment Binding Peptide
RTAPTTPLLLSL

SEQ ID NO: 9 - protein, 12 aa, Artificial Sequence
  Pigment Binding Peptide
WHLSWSPVPLPT

SEQ ID NO: 10 - protein, 7 aa, Artificial Sequence
  Pigment Binding Peptide
PHARLVG

SEQ ID NO: 11 - protein, 7 aa, Artificial Sequence
  Pigment Binding Peptide
NIPYHHP

SEQ ID NO: 12 - protein, 7 aa, Artificial Sequence
  Pigment Binding Peptide
TTMPAIP

SEQ ID NO: 13 - protein, 7 aa, Artificial Sequence
  Pigment Binding Peptide
HNLPPRS

SEQ ID NO: 14 - protein, 12 aa, Artificial Sequence
  Pigment Binding Peptide
AHKTQMGVRQPA

SEQ ID NO: 15 - protein, 12 aa, Artificial Sequence
  Pigment Binding Peptide
ADNVQMGVSHTP

SEQ ID NO: 16 - protein, 12 aa, Artificial Sequence
  Pigment Binding Peptide
AHNAQMGVSHPP

SEQ ID NO: 17 - protein, 12 aa, Artificial Sequence
  Pigment Binding Peptide
ADYVGMGVSHRP

SEQ ID NO: 18 - protein, 7 aa, Artificial Sequence
  Pigment Binding Peptide
YPNTALV

SEQ ID NO: 19 - protein, 7 aa, Artificial Sequence
  Pigment Binding Peptide
VATRIVS

SEQ ID NO: 20 - protein, 12 aa, Artificial Sequence
  Pigment Binding Peptide
HSLKNSMLTVMA

SEQ ID NO: 21 - protein, 7 aa, Artificial Sequence
  Pigment Binding Peptide
NYPTQAP

SEQ ID NO: 22 - protein, 7 aa, Artificial Sequence
  Pigment Binding Peptide
KCCYSVG

SEQ ID NO: 23 - protein, 12 aa, Artificial Sequence
  Pigment Binding Peptide
RHDLNTWLPPVK

SEQ ID NO: 24 - protein, 12 aa, Artificial Sequence
  Pigment Binding Peptide
EISLPAKLPSAS

SEQ ID NO: 25 - protein, 12 aa, Artificial Sequence
  Pigment Binding Peptide
SDYVGMRPSPRH

SEQ ID NO: 26 - protein, 12 aa, Artificial Sequence
  Pigment Binding Peptide
SDYVGMRLSPSQ

SEQ ID NO: 27 - protein, 12 aa, Artificial Sequence
  Pigment Binding Peptide
SVSVGIQPSPRP

SEQ ID NO: 28 - protein, 12 aa, Artificial Sequence
  Pigment Binding Peptide
YVSVGIKPSPRP

SEQ ID NO: 29 - protein, 12 aa, Artificial Sequence
  Pigment Binding Peptide
YVCEGIHPCPRP

SEQ ID NO: 30 - protein, 7 aa, Artificial Sequence
  Print Media Binding Peptide
SILPYPY

SEQ ID NO: 31 - protein, 7 aa, Artificial Sequence
  Cotton Binding Peptide
STASYTR

SEQ ID NO: 32 - protein, 7 aa, Artificial Sequence
  Polyester/Cotton Binding Peptide
LPVRPWT

SEQ ID NO: 33 - protein, 7 aa, Artificial Sequence
  Paper Binding Peptide
GNTPSRA

SEQ ID NO: 34 - protein, 7 aa, Artificial Sequence
  Paper Binding Peptide
YQDSAKT

SEQ ID NO: 35 - protein, 7 aa, Artificial Sequence
  Cellulose Binding Peptide
VPRVTSI

SEQ ID NO: 36 - protein, 7 aa, Artificial Sequence
  Cellulose Binding Peptide
MANHNLS

SEQ ID NO: 37 - protein, 12 aa, Artificial Sequence
  Cellulose Binding Peptide
THKTSTQRLLAA

SEQ ID NO: 38 - protein, 12 aa, Artificial Sequence
  Cellulose Binding Peptide
KCCYVNVGSVFS

SEQ ID NO: 39 - protein, 12 aa, Artificial Sequence
  Cellulose Binding Peptide
AHMQFRTSLTPH

SEQ ID NO: 40 - protein, 12 aa, Artificial Sequence
  Poly(ethylene terephthalate) Binding Peptide
GTSDHMIMPFFN

SEQ ID NO: 41 - DNA, 20 bp, Artificial Sequence
  Primer
ccctcatagttagcgtaagg

SEQ ID NO: 42 - protein, 7 aa, Artificial Sequence
  Control Peptide
SNRDLVY

SEQ ID NO: 43 - protein, 12 aa, Artificial Sequence
  Control Peptide
SSNLNLSWVQDT

SEQ ID NO: 44 - protein, 12 aa, Artificial Sequence
  Control Peptide
KFQNMDRHPASL

SEQ ID NO: 45 - protein, 37 aa, Aspergillus
TSTSKASTTTTSSKTTTTSSKTTTTTSKTSTTSSSST

SEQ ID NO: 46 - DNA, 103 bp, Artificial Sequence
  Oligonucleotide Used to Prepare TBP1 Gene
ggatccatcgaaggtcgtttccacgaaaactggccgtctggtggcggtacctctacttccaaagcttccaccactacgacttctagcaaaaccaccactacat

SEQ ID NO: 47 - DNA, 104 bp, Artificial Sequence
  Oligonucleotide Used to Prepare TBP1 Gene
cctctaagactaccacgactacctccaaaacctctactacctctagctcctctacgggcggtggcactcacaagacctctactcagcgtctgctggctgcataa

SEQ ID NO: 48 - DNA, 69 bp, Artificial Sequence
  Oligonucleotide Used to Prepare TBP1 Gene
ttatgcagccagcagacgctgagtagaggtcttgtgagtgccaccgcccgtagaggagctagaggtagt

SEQ ID NO: 49 - DNA, 69 bp, Artificial Sequence
  Oligonucleotide Used to Prepare TBP1 Gene
agaggttttggaggtagtcgtggtagtcttagaggatgtagtggtggttttgctagaagtcgtagtggt

SEQ ID NO: 50 - DNA, 69 bp, Artificial Sequence
  Oligonucleotide Used to Prepare TBP1 Gene
ggaagctttggaagtagaggtaccgccaccagacggccagttttcgtggaaacgaccttcgatggatcc

SEQ ID NO: 51 - DNA, 207 bp, Artificial Sequence
  TBP1 Gene
ggatccatcgaaggtcgtttccacgaaaactggccgtctggtggcggtacctctacttccaaagcttccaccactacgacttctagcaaaaccaccactacatcctctaagactaccacgactacctccaaaacctctactacctctagctcctctacgggcggtggcactcacaagacctctactcagcgtctgctggctgcataa

SEQ ID NO: 52 - protein, 68 aa, Artificial Sequence
  TBP1 Triblock Protein Dispersant
GSIEGRFHENWPSGGGTSTSKASTTTTSSKTTTTSSKTTTTTSKTSTTSSSSTGGGTHKTSTQRLLAA

SEQ ID NO: 53 - DNA, 22 bp, Artificial Sequence
  Primer
caccggatccatcgaaggtcgt

SEQ ID NO: 54 - DNA, 21 bp, Artificial Sequence
  Primer
tcattatgcagccagcagcgc

SEQ ID NO: 55 - protein, 96 aa, Artificial Sequence
  6H-TBP1 Protein
MSYYHHHHHHLESTSLYKKAGSAAAPFTGSIEGRFHENWPSGGGTSTSKASTTTTSSKTTTTSSKTTTTTSKTSTTSSSSTGGGTHKTSTQRLLAA

SEQ ID NO: 56 - protein, 23 aa, Cellulomonas fimi
PTPTPTPTPTPTPTPTPTPTPTP

SEQ ID NO: 57 - DNA, 90 bp, Artificial Sequence
  Oligonucleotide Used to Prepare TBP2 Gene
ggatccatcgaaggtcgtcactctctgaaaaactccatgctgactgttatggctggtggcggtccgactccaaccccgactccgacgcca

SEQ ID NO: 58 - DNA, 90 bp, Artificial Sequence
  Oligonucleotide Used to Prepare TBP2 Gene
actccgactccgacccctactccgacgccgaccccaactccgggcggtggcggtacctctgaccacatgatcatgccgttcttcaactaa

SEQ ID NO: 59 - DNA, 60 bp, Artificial Sequence
  Oligonucleotide Used to Prepare TBP2 Gene
ttagttgaagaacggcatgatcatgtggtcagaggtaccgccaccgcccggagttggggt

SEQ ID NO: 60 - DNA, 60 bp, Artificial Sequence
  Oligonucleotide Used to Prepare TBP2 Gene
cggcgtcggagtaggggtcggagtcggagttggcgtcggagtcggggttggagtcggacc

SEQ ID NO: 61 - DNA, 60 bp, Artificial Sequence
  Oligonucleotide Used to Prepare TBP2 Gene
gccaccagccataacagtcagcatggagtttttcagagagtgacgaccttcgatggatcc

SEQ ID NO: 62 - DNA, 180 bp, Artificial Sequence
  TBP2 Gene
ggatccatcgaaggtcgtcactctctgaaaaactccatgctgactgttatggctggtggcggtccgactccaaccccgactccgacgccaactccgactccgacccctactccgacgccgaccccaactccgggcggtggcggtacctctgaccacatgatcatgccgttcttcaactaa

SEQ ID NO: 63 - protein, 59 aa, Artificial Sequence
  TBP2 Protein Triblock Dispersant
GSIEGRHSLKNSMLTVMAGGGPTPTPTPTPTPTPTPTPTPTPTPGGGGTSDHMIMPFFN

SEQ ID NO: 64 - DNA, 22 bp, Artificial Sequence
  Primer
caccggatccatcgaaggtcgt

SEQ ID NO: 65 - DNA, 23 bp, Artificial Sequence
  Primer
tcattagttgaagaacggcatga

SEQ ID NO: 66 - protein, 87 aa, Artificial Sequence
  6H-TBP2 Protein
MSYYHHHHHHLESTSLYKKAGSAAAPFTGSIEGRHSLKNSMLTVMAGGGPTPTPTPTPTPTPTPTPTPTPTPGGGGTSDHMIMPFFN

SEQ ID NO: 67 - DNA, 48 bp, Artificial Sequence
  Oligonucleotide used to make pGSTf plasmids
ggccgcctggcatttgtcgtggtctcctgttcctttgcctacttaagg

SEQ ID NO: 68 - DNA, 54 bp, Artificial Sequence
  Oligonucleotide used to make pGSTf plasmid
cgcgccttaagtaggcaaaggaacaggagaccacgacaaatgccaggcggccgc

SEQ ID NO: 69 - DNA, 48 bp, Artificial Sequence
  Oligonucleotide used to make pGSTf plasmids
ggccgcccggactgcgcctactacgccgcttcttctgtcgctttaagg

SEQ ID NO: 70 - DNA, 54 bp, Artificial Sequence
  Oligonucleotide used to make pGSTf plasmids
cgcgccttaaagcgacagaagaagcggcgtagtaggcgcagtccgggcggccgc

SEQ ID NO: 71 - DNA, 33 bp, Artificial Sequence
  Oligonucleotide used to make pGSTf plasmids
ggccgcctttcatgagaattggccttcgtaagg

SEQ ID NO: 72 - DNA, 39 bp, Artificial Sequence
  Oligonucleotide used to make pGSTf plasmids
cgcgccttacgaaggccaattctcatgaaaggcggccgc

SEQ ID NO: 73 - DNA, 33 bp, Artificial Sequence
  Oligonucleotide used to make pGSTf plasmids
ggccgccatgcctccgccgttgatgcagtaagg

SEQ ID NO: 74 - DNA, 39 bp, Artificial Sequence
  Oligonucleotide used to make pGSTf plasmids
cgcgccttactgcatcaacggcggaggcatggcggccgc

SEQ ID NO: 75 - DNA, 33 bp, Artificial Sequence
  Oligonucleotide used to make pGSTf plasmids
ggccgcctatccgaatactgcgttggtgtaagg

SEQ ID NO: 76 - DNA, 39 bp, Artificial Sequence
  Oligonulceotide used to make pGSTf plasmids
cgcgccttacaccaacgcagtattcggataggcggccgc

SEQ ID NO: 77 - DNA, 48 bp, Artificial Sequence
  Oligonucleotide used to make pGSTf plasmids
ggccgcccattcgcttaagaattcgatgcttactgtgatggcgtaagg

SEQ ID NO: 78 - DNA, 54 bp, Artificial Sequence
  Oligonucleotide used to make pGSTf plasmids
cgcgccttacgccatcacagtaagcatcgaattcttaagcgaatgggcggccgc

SEQ ID NO: 79 - DNA, 33 bp, Artificial Sequence
  Oligonucleotide used to make pGSTf plasmids
ggccgccgttgctacgaggattgtgtcttaagg

SEQ ID NO: 80 - DNA, 39 bp, Artificial Sequence
  Oligonucleotide used to make pGSTf plasmids
cgcgccttaagacacaatcctcgtagcaacggcggccgc

SEQ ID NO: 81 - DNA, 33 bp, Artificial Sequence
  Oligonucleotide used to make pGSTf plasmids
ggccgccaatattccttatcatcatccgtaagg

SEQ ID NO: 82 - DNA, 39 bp, Artificial Sequence
  Oligonucleotide used to make pGSTf plasmids
cgcgccttacggatgatgataaggaatattggcggccgc

SEQ ID NO: 83 - DNA, 33 bp, Artificial Sequence
  Oligonucleotide used to make pGSTf plasmids
ggccgccccgcatgctcgtttggtgggttaagg

SEQ ID NO: 84 - DNA, 39 bp, Artificial Sequence
  Oligonucleotide used to make pGSTf plasmids
cgcgccttaacccaccaaacgagcatgcggggcggccgc

SEQ ID NO: 85 - DNA, 48 bp, Artificial Sequence
  Oligonucleotide used to make pGSTf plasmids
ggccgccgcgcataagacgcagatgggtgtgaggcagccggcttaagg

SEQ ID NO: 86 - DNA, 54 bp, Artificial Sequence
  Oligonucleotide used to make pGSTf plasmids
cgcgccttaagccggctgcctcacacccatctgcgtcttatgcgcggcggccgc

SEQ ID NO: 87 - DNA, 48 bp, Artificial Sequence
  Oligonulceotide used to make pGSTf plasmids
ggccgcctctgtttctgtgggtatgaagccgagtcctaggccttaagg

SEQ ID NO: 88 - DNA, 54 bp, Artificial Sequence
  Oligonucleotide used to make pGSTf plasmids
cgcgccttaaggcctaggactcggcttcatacccacagaaacagaggcggccgc

SEQ ID NO: 89 - DNA, 33 bp, Artificial Sequence
  Oligonucleotide used to make pGSTf plasmids
ggccgccaagtgttgttattctgtggggtaagg

SEQ ID NO: 90 - DNA, 39 bp, Artificial Sequence
  Oligonucleotide used to make pGSTf plasmid.
cgcgccttaccccacagaataacaacacttggcggccgc

SEQ ID NO: 91 - DNA, 33 bp, Artificial Sequence
  Oligonucleotide used to make pGSTf plasmids
ggccgccaattatcctacgcaggctccgtaagg

SEQ ID NO: 92 - DNA, 39 bp, Artificial Sequence
  Oligonucleotide used to make pGSTf plasmids
cgcgccttacggagcctgcgtaggataattggcggccgc

SEQ ID NO: 93 - DNA, 48 bp, Artificial Sequence
  Oligonucleotide used to make pGSTf plasmids
ggccgccgagatttcgcttcctgctaagctgcctagtgcttcgtaagg

SEQ ID NO: 94 - DNA, 54 bp, Artificial Sequence
  Oligonucleotide used to make pGSTf plasmids
cgcgccttacgaagcactaggcagcttagcaggaagcgaaatctcggcggccgc

SEQ ID NO: 95 - DNA, 48 bp, Artificial Sequence
  Oligonucleotide used to make pGSTf plasmids
ggccgcctatgtgtgtgaggggattcatccatgtcctaggccttaagg

SEQ ID NO: 96 - DNA, 54 bp, Artificial Sequence
  Oligonucleotide used to make pGSTf plasmids
cgcgccttaaggcctaggacatggatgaatcccctcacacacataggcggccgc

SEQ ID NO: 97 - DNA, 33 bp, Artificial Sequence
  Oligonucleotide used to make pGSTf plasmids
ggccgcctctattctgccgtatccttattaagg

SEQ ID NO: 98 - DNA, 39 bp, Artificial Sequence
  Oligonucleotide used to make pGSTf plasmids
cgcgccttaataaggatacggcagaatagaggcggccgc

SEQ ID NO: 99 - DNA, 33 bp, Artificial Sequence
  Oligonuleotide used to make pGSTf plasmids
ggccgcctcgacggcgtcttatactcgttaagg

SEQ ID NO: 100 - DNA, 39 bp, Artificial Sequence
  Oligonulcoetide used to make pGSTf plasmids
cgcgccttaacgagtataagacgccgtcgaggcggccgc

SEQ ID NO: 101 - DNA, 33 bp, Artificial Sequence
  Oligonucleotide used to make pGSTf plasmids
ggccgcccttccggtgcgtccgtggacttaagg

SEQ ID NO: 102 - DNA, 39 bp, Artificial Sequence
  Oligonucleotide used to make pGSTf plasmids
cgcgccttaagtccacggacgcaccggaagggcggccgc

SEQ ID NO: 103 - DNA, 33 bp, Artificial Sequence
  Oligonucleotide used to make pGSTf plasmids
ggccgcctatcaggattctgcgaaaacgtaagg

SEQ ID NO: 104 - DNA, 39 bp, Artificial Sequence
  Oligonucleotide used to make pGSTf plasmids
cgcgccttacgttttcgcagaatcctgataggcggccgc

SEQ ID NO: 105 - DNA, 48 bp, Artificial Sequence
  Oligonucleotide used to make pGSTf plasmids
ggccgccaagtgttgttatgtgaatgttgggtctgttttttcttaagg

SEQ ID NO: 106 - DNA, 54 bp, Artificial Sequence
  Oligonucleotide used to make pGSTf plasmids
cgcgccttaagaaaaaacagacccaacattcacataacaacacttggcggccgc

SEQ ID NO: 107 - DNA, 33 bp, Artificial Sequence
  Oligonucleotide used to make pGSTf plasmids
ggccgccgttccgcgtgttacttcgatttaagg

SEQ ID NO: 108 - DNA, 39 bp, Artificial Sequence
  Oligonucleotide used to make pGSTf plasmids
cgcgccttaaatcgaagtaacacgcggaacggcggccgc

SEQ ID NO: 109 - DNA, 33 bp, Artificial Sequence
  Oligonucleotide used to make pGSTf plasmids
ggccgccatggcgaatcataatctttcttaagg

SEQ ID NO: 110 - DNA, 39 bp, Artificial Sequence
  Oligonucleotide used to make pGSTf plasmids
cgcgccttaagaaagattatgattcgccatggcggccgc

SEQ ID NO: 111 - DNA, 58 bp, Artificial Sequence
  Oligonucleotide used to make pGSTf plasmids
catggtccgcggccgccactcacaagacctctactcagcgtctgctggctgcataagg

SEQ ID NO: 112 - DNA, 58 bp, Artificial Sequence
  Oligonucleotide used to make pGSTf plasmids
cgcgccttatgcagccagcagacgctgagtagaggtcttgtgagtggcggccgcggac

SEQ ID NO: 113 - DNA, 48 bp, Artificial Sequence
  Oligonucleotide used to make pGSTf plasmids
ggccgccggtacgtcggatcatatgattatgcctttttttaattaagg

SEQ ID NO: 114 - DNA, 54 bp, Artificial Sequence
  Oligonucleotide used to make pGSTf plasmids
cgcgccttaattaaaaaaaggcataatcatatgatccgacgtaccggcggccgc

SEQ ID NO: 115 - protein, 12 aa, Artificial Sequence
  Cotton-Binding Peptide
KPPPPSILPYPY

SEQ ID NO: 116 - DNA, 210 bp, Artificial Sequence
  Coding region of pENTR-TBP101 plasmid
ggatccatcgaaggtcgtttccacgaaaactggccgtctgccggcggtacctctacttccaaagcttccaccactacgacttctagcaaaaccaccactacatcctctaagactaccacgactacctccaaaacctctactacctctagctcctctacgggcggcgccactcacaagacctctactcagcgtctgctggctgcataatga

SEQ ID NO: 117 - protein, 4 aa, Artificial Sequence
  Xa recognition site
IEGR

SEQ ID NO: 118 - protein, 68 aa, Artificial Sequence
  Protein encoded by pENTR-TBP101
GSIEGRFHENWPSAGGTSTSKASTTTTSSKTTTTSSKTTTTTSKTSTTSSSSTGGATHKTSTQRLLAA

SEQ ID NO: 119 - DNA, 168 bp, Artificial Sequence
  Coding region of pENTR-TBP201 plasmid
ggatccatcgaaggtcgtttccacgaaaactggccgtctgccggcggtccgactccaaccccgactccgacgccaactccgactccgacccctactccgacgccgaccccaactccgggcggcgccactcacaagacctctactcagcgtctgctggctgcataatga

SEQ ID NO: 120 - protein, 54 aa, Artificial Sequence
  Protein encoded by pENTR-TBP201
GSIEGRFHENWPSAGGPTPTPTPTPTPTPTPTPTPTPTPGGATHKTSTQRLLAA

SEQ ID NO: 121 - DNA, 5562 bp, Artificial Sequence
  pINK251 plasmid
atcgagatctcgatcccgcgaaattaatacgactcactatagggagaccacaacggtttccctctagaaataattttgtttaactttaagaaggagatatacatatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggctccgcggccgcccccttcaccggatccatcgaaggtcgtttccacgaaaactggccgtctgccggcggtccgactccaaccccgactccgacgccaactccgactccgacccctactccgacgccgaccccaactccgggcggcgccactcacaagacctctactcagcgtctgctggctgcataatgaaagggtgggcgcgccgacccagctttcttgtacaaagtggtttgattcgacccgggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagagg

SEQ ID NO: 122 - protein, 298 aa, Artificial Sequence
  Protein encoded by pINK251
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGSAAAPFTGSIEGRFHENWPSAGGPTPTPTPTPTPTPTPTPTPTPTPGGATHKTSTQRLLAA